SEQUENCE LISTING (15 sequences)

SEQ ID NO: 1 - RNA, 25 nt, Homo sapiens
uauacacgccgcaauacagagacuc

SEQ ID NO: 2 - RNA, 25 nt, Homo sapiens
gagucucuguauugcggcguguaua

SEQ ID NO: 3 - RNA, 25 nt, Homo sapiens
uagaagucacaauuaaaccaaggcc

SEQ ID NO: 4 - RNA, 25 nt, Homo sapiens
ggccuugguuuaauugugacuucua

SEQ ID NO: 5 - protein, 9 aa, Unknown
  CMVPP65
NLVPMVATV

SEQ ID NO: 6 - protein, 9 aa, Unknown
  hTERT
ILAKFLHWL

SEQ ID NO: 7 - protein, 9 aa, Unknown
  EBV BMLF1
GLCTLVAML

SEQ ID NO: 8 - DNA, 786 bp, Homo sapiens
  CDS (1)..(783)
atgtcttggaaaaaggctttgcggatccccggaggccttcgggtagcaactgtgaccttgatgctggcgatgctgagcacctcggtggctgagggcagagactctcccgaggatttcgtgtaccagtttaagggcatgtgctacttcaccaacgggacggagcgcgtgcgtcttgtgaccagatacatctataaccgagaggagtacgcacgcttcgacagcgacgtgggggtgtaccgggcggtgacgccgctggggccgcctgacgccgagtactggaacagccagaaggaagtcctagaggggacccgggcggagttggacacggtgtgcagacacaactaccagttggagctccgcacgaccttgcagcggcgagtggagcccacagtgaccatctccccatccaggacagaggccctcaaccaccacaacctgctggtctgctcagtgacagatttctatccagcccagatcaaagtccggtggtttcggaatgatcaggaggagacaaccggcgttgtgtccacccccctcattaggaacggtgactggaccttccagatcctggtgatgctggaaatgactccccagcgtggagatgtctacacctgccacgtggagcaccccagcctccagacccccatcaccgtggagtggcgggctcagtctgaatctgcccagagcaagatgctgagtggcattggaggcttcgtgctggggctaatcttcctcgggctgggccttattatccatcacaggagtcagaaagggctcctgcactga

SEQ ID NO: 9 - protein, 261 aa, Homo sapiens [sequence omitted]

SEQ ID NO: 10 - DNA, 5391 bp, Homo sapiens
  CDS (71)..(152)
  CDS (3850)..(4098)
  CDS (4544)..(4825)
  CDS (5173)..(5324)
cttgtcttgaggccctcacaattgctctacagctcagaacagcaactgctgaggctgccttgggaagaggatgatcctaaacaaagctctgatgctgggggccctcgccctgaccaccgtgatgagcccttgtggaggtgaagacattgtgggtgagtgcatgagtgaggaatgttctctggagctgaaaaacagtaaattgaaggaaaagagagaaagcgatttgcagagaaattgtagagatttcctaagacccctttcagtattaagagaattaaaaattatagctgttcctccttcaggaaaccagagccccaacctactctttttgttatgtatgcttttgtgttcactaaggatgctattctgtttatattatattcagtgacaacagcctggaggtctctatgtcgttccgtcatgattgcctcaaaaattagtgaagtttccatcagtggataattttttattattaaaaatgtatgaagtgtcattctcaaatttccctgaacaacttttgaagcttttcgtatgtctcctgtagtagatcttggggtcgttccatcaattatatactctatagatattaaaaaagttgcccgtttctttctctcagacttactcacatttccacatgggaactggcacaggtggggagtgggtaaaggagtccagcaggctgaatgccttcaacaatcattttaccacatggtcctcacttactctcagctgcctcatatgtgtcacctcacaaataatcaaataaaatgggcatgtagctaagctttgtaaatagtgaaaacatggatgtcaattgtttttacatatttctattacaggtatagcttcacatttttctttagcaaaataagggatccttttagtttaaaattgagaagtagaaaaaattggtaaattaaatcattttattctcaaattatcaacccaaattacctgttcttcacctcatctaataaagtcctataaaaagaaaagtgggccagacatggtggctcatgcctgtaatcccagcactttgggaggccgaagcaggaggatcatttgagcctgggagtttgagaccagcctgggcaacatagcaagacctcatctctaccaaaaaataaaataaaaattagccaggtgtggtggtgcatgcctgtggtgccagctactcagaaggctgcagtgggaggagcacttgagtccaggaggtggaagctgcagtgagccatgatggcaccactacactccagccagggcaacagagagagactctgtctcaaaaagaaagaggaaagaaagagagaaaggaaggaaagaaggaaagaaggaaggaaggaaggagaaagggaagggaggaaggaaaaaagaaagaaagaaagaaaacggaaggaaggaagcacagattaattatttggtctctttgtctcctctgcctttgtcgtccatctcttcccacctctcttcatgcattcctttctccctcttccctttcaggatccatctctgactccctgctcctttatagagatggacagtgagtttgtaaaacaaaagttgaaaagtcagatagttaaaaggggaagtgaactggaaggtactctaaactttcacaaccttattaaccatggctgctcccattctgattttgttcagcagtggaagtttcacccgctcctccagagcgcttggcttctttgttccaaatttcctttcttcaacctcacaccagagtgccctggtcaggctcagctcatccattaggcacaatgtgggcagtgcaggggaccctccagactgtaaagccacatgagaatgttttaactccttttaaaattataaaaaaatgaaattgtagagcctaagaaaatgttttaacttttaattcagcctagattatattgtctttataccaattcagtcataaaatatagttttccatatttttatggaggaaggcgtccacacaagcaagagtgcttggggctcacatgtcagaacgcaaccctgatcatggctgatcctggccttcgtgtggttctgctaactatgtgcctgtcagtcttccccaaaatctatgtggtcctcaaatataacaactgtcattcaatacacatgtttgagcacccagtgagctaagttttaaggattcaaagatgaaaagtcatgctgtctcctctgcaaagggtgctcagactagtgatggaaacagtatgggatgaaagaaagcagaaggccattgctgagcaggcagtggactcagcagaggctgaaactatacaagtgacttggttccagctgggccagcaggataaccagatgaaaagaaggattgcatatattccatatatatttatgtttgaacaaagagtcaaggtttattgcaaggataaggaggttttgttggtggcctgttaagaccatccagggtggtcatactggatagggaagaaggtgagctggaagaggaacagacaaacttggatggccagatgttgagatggaggagatggaggtcataacgtggtcaaaaacatgttgatgagaggacttagctacaaagttgttaacttaagcagaaacctcaaggattgattttatgatttctccaggaagtcctaaaagttaatttcatttcagggagaaaaacaacagaccactgcaaagaccaggaacatgaaaggataatgtagtttggtttgcttggcagatacttgtgaaagatgttggactgtaaggctgtcaatatcctcctcgcagaacttactacagtacattgtatctgctcccttacctacctgactctcccactattcagtttgttccttaatggtagaccatgcctgatcggtgttttacacatcccctgctatgtctgatacttgtggatgctcagaaagtggggaaggaaggaaagatacgatggtaaaaggcttacacatgtcttgagcagaatgttcagtttggctcatttggctggagtcatactgcatggctgccattctgctctggcatcctcagagaagcacactgcccattaaaggaaaaagggtgaatacaaatgttgagtcagaacactgcagacatttagtaacctccttcagaggaaaaaaaaaggtggggggaatgacagaaatccaaaaactagtagagcttccactttttcatttcagaagaaatcagttactctcctctaaggaccattactattaacaaaacagagaccttagaaggaagcattatttacttatcatatattttgtaatgttattacccttcttgttatactctttcttataccctaccattgttagcagaaattattttaaattaataagatcctgcatgcttttcctttttctaaaaaaagaaagatctctgtgtagaatgtcctgttctgagccagtcctgagaggaaaggaagtataatcaatttgttattaactgatgaaagaattaagtgaaagataaaccttaggaagcagagggaagttaatctatgactaagaaagttaagtactctgataactcattcattccttcttttgttcatttacattatttaatcacaagtctatgatgtgccaggcactcaggaaatagtgaaaattggacacgcgatattctgcccttgtgtagcacacaccgtagtgggaaagaaagtgcacttttaaccggacaactatcaacacgaagaggggaggaagcaggggctggaaatgtccacagactttgccaaagacaaagcccataatatctgaaagtcagtttcttccatcattttgtgtattaaggttctttattcccctgttctctgccttcctgcttgtcatcttcactcatcagctgaccatgttgcctcttacggtgtaaacttgtaccagtcttatggtccctctgggcagtacagccatgaatttgatggagacgaggagttctatgtggacctggagaggaaggagactgtctggcagttgcctctgttccgcagatttagaagatttgacccgcaatttgcactgacaaacatcgctgtgctaaaacataacttgaacatcgtgattaaacgctccaactctaccgctgctaccaatggtatgtgtccaccattctgcctttctttactgatttatccctttataccaagtttcattattttctttccaagaggtccccagatcttctcatggcaattgctgaaattttatcatttctcatctctaaaatcacatattccaatgtaatacaagggtctttccattatgcattcattaaatccttctaggagaggtctcatcaaccttctactttattaaacatgcccacagagagaagggcacaggagtaaagcagaggcaatgtgtcgttgctcccaaatgtgtcgttacaatgtgtcgttgcttacccaaagaggtaaataaggcctctttgaccagcaggagaggaaatgctggtaggaagactcttccaggatgtaatgcagaagaagctcagggcagagctattcacactttacaccagtgctgtttcctcaccatagaggttcctgaggtcacagtgttttccaagtctcccgtgacactgggtcagcccaacaccctcatctgtcttgtggacaacatctttcctcctgtggtcaacatcacctggctgagcaatgggcactcagtcacagaaggtgtttctgagaccagcttcctctccaagagtgatcattccttcttcaagatcagttacctcaccttcctcccttctgctgatgagatttatgactgcaaggtggagcactggggcctggatgagcctcttctgaaacactggggtaaggatgagtttcaccattttttgatgctttcttgtctgtcaagttcagaacttcctgccttttactctatatcccaaaacttgttttccacacttcatgagtttcttttgtcttttttttgaaagaattaagcaacaaaagcacagatttattaaaaaagaaagtacactccacagggtgggagcaggcctgccacttcatgggtttctaataacagacttcactctcctccctaagctgggggccttgagtctttgcagagccaaccctctaccccatcccatcccacacacatgcacatgagcaaactctgcattctgacctcaacaacttcacttccacagagcctgagattccaacacctatgtcagagctcacagagactgtggtctgcgccctggggttgtctgtgggcctcgtgggcattgtggtggggaccgtcttgatcatccgaggcctgcgttcagttggtgcttccagacaccaagggcccttgtgaatcccatcctgaaaaggaaggtaagattgagatttgttagagctgaagctgcaggaaggaaagt

SEQ ID NO: 11 - protein, 255 aa, Homo sapiens
MILNKALMLGALALTTVMSPCGGEDIVADHVASYGVNLYQSYGPSGQYSHEFDGDEEFYVDLERKETVWQLPLFRRFRRFDPQFALTNIAVLKHNLNIVIKRSNSTAATNEVPEVTVFSKSPVTLGQPNTLICLVDNIFPPVVNITWLSNGHSVTEGVSETSFLSKSDHSFFKISYLTFLPSADEIYDCKVEHWGLDEPLLKHWEPEIPTPMSELTETVVCALGLSVGLVGIVVGTVLIIRGLRSVGASRHQGPL

SEQ ID NO: 12 - DNA, 819 bp, Homo sapiens
  CDS (28)..(792)
actcccaaaagagcgcccaagaagaaaatggccataagtggagtccctgtgctaggatttttcatcatagctgtgctgatgagcgctcaggaatcatgggctatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacttggaaatcatgacaaagcgctccaactatactccgatcaccaatgtacctccagaggtaactgtgctcacgaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgtggtgtgtgccctgggcctgactgtgggtctggtgggcatcattattgggaccatcttcatcatcaagggattgcgcaaaagcaatgcagcagaacgcagggggcctctgtaaggcacatggaggtgatggtgtttctta

SEQ ID NO: 13 - protein, 254 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLRKSNAAERRGPL

SEQ ID NO: 14 - DNA, 1158 bp, Homo sapiens
  CDS (44)..(844)
cttgcctgcttctctggcccctggtcctgtcctgttctccagcatggtgtgtctgaagctccctggaggctcctgcatgacagcgctgacagtgacactgatggtgctgagctccccactggctttgtctggggacacccgaccacgtttcctgtggcagcctaagagggagtgtcatttcttcaatgggacggagcgggtgcggttcctggacagatacttctataaccaggaggagtccgtgcgcttcgacagcgacgtgggggagttccgggcggtgacggagctggggcggcctgacgctgagtactggaacagccagaaggacatcctggagcaggcgcgggccgcggtggacacctactgcagacacaactacggggttgtggagagcttcacagtgcagcggcgagtccaacctaaggtgactgtatatccttcaaagacccagcccctgcagcaccacaacctcctggtctgctctgtgagtggtttctatccaggcagcattgaagtcaggtggttcctgaacggccaggaagagaaggctgggatggtgtccacaggcctgatccagaatggagactggaccttccagaccctggtgatgctggaaacagttcctcgaagtggagaggtttacacctgccaagtggagcacccaagcgtgacaagccctctcacagtggaatggagagcacggtctgaatctgcacagagcaagatgctgagtggagtcgggggctttgtgctgggcctgctcttccttggggccgggctgttcatctacttcaggaatcagaaaggacactctggacttcagccaacaggattcctgagctgaaatgcagatgaccacattcaaggaagaactttctgccccggctttgcaggatgaaagctttcctgcttggcagttattcttccacaagagagggctttctcaggacctggttgctactggttcggcaactgcagaaaatgtcctcccttgtggcttcctcagctcctgcccttggcctgaagtcccagcattgatggcagcgcctcatcttcaacttttgtgctcccctttgcctaaaccgtatggcctcccgtgcatctgtattcaccctgtatgacaaacacattacattattaaatgtttctcaaagatgg

SEQ ID NO: 15 - protein, 266 aa, Homo sapiens
MVCLKLPGGSCMTALTVTLMVLSSPLALSGDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

(19) United States
(12) Patent Application Publication
(10) Pub. No.: US 2016/0058793 A1
(43) Pub. Date: Mar. 3, 2016

(54) Tumor Targeted Sickle Erythroid Precursors, Progenitors and Hematopoietic Stem Cells, Induced Pluripotent Stem Cells, Microparticles and Liposomes for Treatment of Cancer

(71) Applicant: David S. Terman, Pebble Beach, CA (US)

(72) Inventor: David S. Terman, Pebble Beach, CA (US)

(21) Appl. No.: 14/800,516

(22) Filed: Jul. 15, 2015

Related U.S. Application Data
(63) Continuation-in-part of application No. 14/222,292, filed on Mar. 21, 2014.
Application No. 14/222,292 is a continuation-in-part of application No. 13/367,797, filed on Feb. 7, 2012, now Pat. No. 8,431,117.
Application No. 13/367,797 is a continuation-in-part of application No. 12/586,532, filed on Sep. 22, 2009, now abandoned.
Application No. 12/586,532 is a continuation-in-part of application No. 12/276,941, filed on Nov. 24, 2008, now Pat. No. 8,524,218.
Application No. 12/276,941 is a continuation-in-part of application No. 12/145,949, filed on Jun. 25, 2008, now Pat. No. 7,803,637.
Application No. 12/145,949 is a division of application No. 10/937,758, filed on Sep. 8, 2004, now abandoned.
Application No. 10/937,758 is a continuation of application No. 09/650,884, filed on Aug. 30, 2000, now abandoned.
Application No. 09/650,884 is a continuation-in-part of application No. 14/037,176, filed on Sep. 25, 2013.
Application No. 14/037,176 is a continuation-in-part of application No. 13/317,590, filed on Oct. 20, 2011, now abandoned.
Application No. 13/317,590 is a continuation-in-part of application No. 13/328,748, filed on Dec. 16, 2011, now Pat. No. 9,149,504.
Application No. 13/328,748 is a continuation-in-part of application No. 13/317,590, filed on Oct. 20, 2011, now abandoned.
Application No. 14/037,176 is a continuation-in-part of application No. 12/526,532, filed on Aug. 10, 2009, now abandoned.
Application No. 12/526,532 is a continuation-in-part of application No. 13/367,797, filed on Feb. 7, 2012, now Pat. No. 8,431,117.
Application No. 13/367,797 is a continuation-in-part of application No. 12/586,532, filed on Sep. 22, 2009, now abandoned.
(60) Provisional application No. 62/133,824, filed on Mar. 16, 2015.
(60) Provisional application No. 62/054,231, filed on Sep. 23, 2014.
(60) Provisional application No. 62/025,396, filed on Jul. 16, 2014.
(60) Provisional application No. 60/151,470, filed on Aug. 30, 1999.
(60) Provisional application No. 61/455,592, filed on Oct. 20, 2010.
(60) Provisional application No. 61/455,592, filed on Oct. 20, 2010.
(60) Provisional application No. 61/807,457, filed on Apr. 2, 2013.
(60) Provisional application No. 61/215,906, filed on May 11, 2009.
(60) Provisional application No. 61/211,227, filed on Mar. 28, 2009.
(60) Provisional application No. 61/206,338, filed on Jan. 28, 2009.
(60) Provisional application No. 61/205,776, filed on Jan. 22, 2009.
(60) Provisional application No. 61/192,949, filed on Sep. 22, 2008.

(51) Int. Cl. A61K 35/17; C12N 5/0786; C12N 5/078; C12N 5/0783; A61K 35/14; A61K 39/00

(52) U.S. Cl. CPC A61K 35/17; A61K 35/14; A61K 39/0011; C12N 5/0634; C12N 5/0636; C12N 5/0645; A61K 2039/5154; C12N 2510/00; C12N 2502/1157; C12N 2502/1114

(57) ABSTRACT

The present invention contemplates a method of treating cancer comprising CD47 deficient sickle cell erythroid precursors/progenitors or hematopoietic stem cells transduced with viral altered genomic DNA from normal or tumor bearing hosts and used to polarize macrophages and/or activate a cytotoxic T cell tumoricidal response.

### CROSS REFERENCE TO RELATED DOCUMENTS

The present application claims priority to U.S. provisional application 62/133,824 filed on Mar. 16, 2015, provisional application 62/054,231 filed on Sep. 23, 2014, and provisional application 62/025,396 filed Jul. 16, 2014

The present application is a continuation in part of U.S. patent application Ser. No. 14/222,292 filed Mar. 21, 2014 which is a continuation in part of U.S. patent application Ser. No. 13/367,797 filed Feb. 7, 2012 which is a continuation in part of Ser. No. 12/586,532 filed Sep. 22, 2009 (abandoned) which is a continuation in part of U.S. patent application Ser. No. 12/276,941 filed Nov. 24, 2008, which is a continuation in part of Ser. No. 12/145,949 filed Jun. 25, 2008 (abandoned) which issued as U.S. Pat. No. 7,803,637 on Sep. 28, 2010 which is a divisional of U.S. patent application Ser. No. 10/937,758 filed Sep. 8, 2004 (abandoned) which is a continuation of U.S. patent application Ser. No. 09/650,884 filed Aug. 30, 2000 (abandoned) which is a continuation of U.S. provisional patent application 60/151,470 filed Aug. 30, 1999 (abandoned). All of the above patents and patent applications and their references are incorporated by reference in their entirety.

The present application is also a continuation in part of U.S. patent application Ser. No. 14/037,176 filed on Sep. 25, 2013 which is a continuation in part of U.S. patent application Ser. No. 13/317,590 filed Oct. 20, 2011 which claims priority to provisional U.S. patent application 61/455,592 filed Oct. 20, 2010 (abandoned). Both of these applications are incorporated by reference.

The present application is also a continuation in part of U.S. patent application Ser. No. 13/328,748 filed Dec. 16, 2011 which is a continuation in part of U.S. patent application Ser. No. 13/317,590 filed Oct. 20, 2011 which is a continuation in part of U.S. provisional application Ser. No. 61/455,592 filed Oct. 20, 2010 which is a continuation in part of U.S. patent application Ser. No. 12/586,532 filed Sep. 22, 2009. All of these patents and patent applications are incorporated in entirety by reference with their references.

The present application claims priority U.S. provisional application 61/807,457 filed Apr. 2, 2013, U.S. patent application Ser. No. 13/367,797 filed Feb. 7, 2012, U.S. patent application Ser. No. 12/586,532 filed Sep. 22, 2009 (abandoned) and also claims priority to U.S. provisional application Ser. No. 61/215,906 filed May 11, 2009 (abandoned) and U.S. provisional application Ser. No. 61/211,227 filed Mar. 28, 2009 (abandoned) and U.S. provisional application Ser. No. 61/206,338 filed on Jan. 28, 2009 (abandoned) and U.S. provisional application Ser. No. 61/205,776 filed Jan. 22, 2009 (abandoned) and U.S. provisional application Ser. No. 61/192,949 filed on Sep. 22, 2008 (abandoned). All of the above patents and patent applications and their references are incorporated by reference in their entirety.

The following applications are related and incorporated by reference: PCT/US07/69869 filed May 29, 2007 (abandoned) and U.S. provisional application Ser. No. 60/809,553 filed on May 30, 2006 (abandoned) and U.S. provisional application Ser. No. 60/819,551 filed on Jul. 8, 2006 (abandoned) and U.S. provisional application Ser. No. 60/842,213 filed on Sep. 5, 2006 (abandoned) and U.S. patent application Ser. No. 10/428,817, filed May 5, 2003 (abandoned) and U.S. provisional application Ser. No. 60/438,686, filed Jan. 9, 2003 (abandoned) and U.S. provisional application Ser. No. 60/415,310, filed on Oct. 1, 2002 (abandoned) and U.S. provisional application Ser. No. 60/406,750, filed on Aug. 29, 2002 (abandoned) and U.S. provisional application Ser. No. 60/415,400, filed on Oct. 2, 2002 (abandoned) and U.S. provisional application Ser. No. 60/406,697, filed on Aug. 28, 2002 (abandoned) and U.S. provisional application Ser. No. 60/389,366, filed on Jun. 15, 2002 (abandoned) and U.S. provisional application Ser. No. 60/378,988, filed on May 8, 2002 (abandoned) and U.S. patent application Ser. No. 09/870,759 filed on May 30, 2001 (abandoned) and U.S. patent application Ser. No. 09/650,884 filed Aug. 30, 2000 (abandoned) and U.S. provisional patent application Ser. No. 60/151,470 filed on Aug. 30, 1999 (abandoned).

### FIELD OF THE INVENTION

The invention is in the fields of genetics and medicine and covers compositions and methods for targeted delivery sickled erythrocytes, their nucleated precursors, microparticles derived therefrom and liposomes expressing phosphatidylserine and heme for treatment of cancer.

### BACKGROUND

Resistance of hypoxic niches, present in most solid tumors to chemotherapy and radiotherapy remains a major cause of tumor recurrence. Treatment of such refractory niches calls for conceptually new approaches. Under hypoxic conditions within tumors, evolutionarily conserved oxygen sensors initiate distress pathways that lead to activation of hypoxia-inducible transcription factors, proinflammatory and pro-angiogenic stimuli. The latter induce a disordered network of blood vessels, anastomotic branches, fenestrations and shunts resulting in heterogeneous blood perfusion, nutrient delivery, cyclic or chronic deoxygenation and aerobic glycolysis. In this microenvironment, tumors exhibit treatment resistance, impaired drug transport and aggressive malignant progression.

Using intravital microscopy in the dorsal skin window and hyperspectral tumor imaging, our prior published work exploited a hitherto unrecognized ability of sickle red blood cells (SSRBCs) but not normal RBCs (nRBCs) to rapidly and selectively adhere to tumor microvasculature, form microaggregates and occlude up to 88% of tumor microvessels. We also showed that SSRBCs but not nRBCs potentiate the tumoricidal effect of exogenous pro-oxidants versus the chemotherapy resistant 4T1 mammary carcinoma. This chain of events pointed to a central role of SSRBC adherence to the tumor endothelium as the initiating event in the tumoricidal process. Indeed, the tumor vasculature was shown to express ligands av integrins, VCAM-1 and laminin-α5 whose cognate receptors are expressed on activated SSRBCs but not nRBCs. Amidst the SSRBC subpopulations expressing such adhesion molecules, the instant invention selects SSRBCs displaying phosphatidylserine receptors as the major cell population mediating adhesion to the tumor microvasculature.

Phosphatidylserine (PS) is normally found on the inner leaflet of the plasma membrane with very little exposure on the outer leaflet of most healthy cells. In contrast to normal erythrocytes, 2-12% of circulating erythrocytes in patients with homozygous sickle cell anemia express PS on their outer leaflet (Setty et al., Blood 111: 905-914 (2008); Kuypers et al., Blood 87: 1179-1187 (1996)). Such SSRBCs are found among the very high and very low density fractions of SSRBCs. PS exposure occurs when SSRBCS undergo natural or induced apoptosis and accompanied by FAS expression along with loss of aminophospholipid translocase APLT activity (Mandal et al., J. Biol. Chem. 280:39460-39467 (2005)). Externalization of PS serves as a signal for phagocytic recognition and removal of apoptotic cells. Importantly, in sickle cell disease there is a positive correlation between the percent PS-positivity and sickle cell—endothelial adhesion. Such adhesion is based on the affinity of PS expressing SSRBCs for matrix thrombospondin and putative PS receptors present on the tumor endothelium (Setty supra (2008); Setty et al., Blood 99: 1564-1571 (2002); Jansen et al., Arterioscler Thromb Vasc Biol. 32:1925-1935 (2012); Hochreiter-Hufford et al., Cold Spring Harb Perspect Biol 2013; 5:a008748 P; Gupta et al., Biochim et Biophys Acta 1453 63-73 (1999); Betal et., Translational Res 152:165-177 (2008); Manodori et al., Blood 95: 1293-1300 (2000); Manodori et al., Microvasc Res 61, 263-274 (2001)). The adhesion of SSRBCs displaying externalized PS to tumor endothelium leads to the formation of heterocellular aggregates by facilitating adhesive interactions with other circulating cellular elements of blood such as platelets and monocytes. The present invention therefore provides SSRBCs and microparticles shed therefrom expressing PS along with liposomes exhibiting PS and heme for treatment of patients with cancer notable for their resistance to existing therapy. There is no prior art directed to the application of this PS exposing subpopulation of SSRBCs, microparticles derived therefrom and liposomes containing PS and heme for treatment of cancer.

### SUMMARY

The present invention contemplates CD47 deficient normal or sickle erythrocyte or erythroblast populations transduced with DNA from tumor cells or normal cells of the same histologic type capable of inducing a macrophage and T cell dependent anti-tumor response.

### FIGURE LEGENDS

FIG. 1: Displayed is the percentage of PS-exposing SSRBCs following exposure to various doses of calcium ionophore.

FIG. 2: Histology of B16 melanoma, lungs and liver following two infusion of PSSSRBCs shows extensive acute necrosis and microhemorrhages in the tumor whereas lung and liver from the same mice showed no such effects. Tumor from untreated mice also showed no necrosis, hemorrhage or thrombosis.

### DEFINITIONS AND ABBREVIATIONS

Erythroid precursor cells also known as “erythroblasts” or cells of the “erythrobastoid series” originate from erythroid progenitor cells and are defined by flow cytometry as CD71+. In the specification erythroid precursor cells are further distinguished by morphologic criteria. The proerythroblast is the earliest erythroid precursor cell and gives rise to the erythroblastoid series. Its progeny includes basophilic erythroblasts followed by polychromatophilic and orthochromatic erythroblasts. Additional definition of Erythroid progenitor cells provided in (Papayannopoulou T, Migliaccio A R Hematology: Basic Principles and Practice, Hoffman, R ed. Elsevior, Chapter 24, 258-279, 2011) which is incorporated by reference in entirety.

Erythroid progenitor cells: These are unipotent (single lineage identity), generally CD34+ derived from multipotent hematopoietic stem cells or embryonic stem cells. Erythroid progenitor cells develop in two phases namely erythroid burst-forming units (BFU-E) followed by erythroid colony-forming units (CFU-E). BFU-E differentiate into CFU-E on stimulation by erythropoietin and then further differentiate into histologically defined erythroblasts or erythroid precursor cells. Additional definition of Erythroid progenitor cells provided in (Papayannopoulou T, Migliaccio A R Hematology: Basic Principles and Practice, Hoffman, R ed. Elsevior, Chapter 24, 258-279, 2011) which is incorporated by reference in entirety. Table 24-1 from Papayannopoulou T, Migliaccio A R supra 2011 with additional structural and functional properties of erythroid progenitor cells is provided below as Table 1.

TABLE A
Changes in the General Properties During Differentiation of
Erythroid Progenitors

| | CFU-GEMM | | |
|---|---|---|---|
| | (CMP) | BFU-E | CFU-E |
| GENERAL FEATURES | | | |
| Self-renewal | ++ | + | 0 |
| Differentiation potential | Multipotent | Erythroid | Erythroid |
| | | committed | committed |
| Cycling status % suicide with ³H | 15-20 | 30-40 | 60-80 |
| thymidine | | | |
| Cell density (g/mL) | <1.077 | <1.077 | <1.077 |
| Incidence/10⁵ cells | 2-5 | 40-120 | 200-600 |
| Circulate in blood | + | + | 0 |
| GROWTH FACTOR RESPONSE | | | |
| EPO | + | + | ++ |
| TPO | + | + | + |
| KL | + | + | − |
| GM-CSF, IL-3 | + | + | − |
| FL | + | 0 | 0 |
| G-CSF, IL-6, IL-1 | + | 0 | 0 |
| Insulin, insulin-like growth factor, | 0 | 0 | + |
| activin | | | |
| TGFβ1 | − | − | ++ |
| Hyper-IL-6 | + | + | + |
| RECEPTOR/ANTIGEN | | | |
| CD34 | ++ | ++ | − |
| CD33 | + | + | 0 |
| C-KIT | ++ | ++ | − |
| HLA-DR (-DP, -DQ) | ++ | ++ | + |
| EPO receptor | + | + | ++ |
| gp130 | + | + | + |
| Tumor necrosis factor receptor | + | + | ++ |
| P67 laminin | − | + | − |
| EP-1¹⁶³ | + | + | ++ |
| 23.6* | 0 | 0 | + |
| CD36 | 0 | ± | + |
| Glycophorin A | 0 | 0 | + |
| ABH, Ii^{†} | 0 | + | + |
| ADHESION MOLECULES | | | |
| VLA4 (CD49d/CD29) | ++ | ++ | ++ |
| VLA5 (CD49e/CD29) | + | + | ++ |
| CD41 | + | + | |
| CD11a/CD18 | + | + | |
| CD44 | + | + | |
| HCAM^{‡} | + | + | |
| TRANSCRIPTION FACTORS | | | |
| GATA2 | ++ | + | − |
| GATA1 | + | ++ | +++ |
| SCL | + | + | + |
| EKLF | + | + | ++ |
| Myb | ++ | + | − |
| Id1, Id2 | ++ | + | − |

BFU-E, Burst-forming unit-erythroid; CFU-E, colony-forming unit-erythroid; CFU-GEMM (CMP), colony-forming unit-granulocyte, erythrocyte, macrophage, megakaryocyte (common myeloid progenitor); EKLF, erythroid Krüppel-like factor; EPO, erythropoietin; FL, Flt-3 ligand; G-CSF, granulocyte colony-stimulating factor; GM-CSF, granulocyte-macrophage colony-stimulating factor; HCAM, homing-associated cytoadhesion molecule; HLA, human leukocyte antigen; IL, interleukin; KL, KIT ligand; SCL, stem cell leukemia; TGF, transforming growth factor; TPO, thrombopoietin.

Erythroblasts: These are nucleated precursor cells in the erythrocytic series. Four developmental and morphological stages in the series are recognized: the proerythroblast, the basophilic erythroblast, in which the cytoplasm is basophilic, the nucleus is large with clumped chromatin, and the nucleoli have disappeared; the polychromatophilic erythroblast., in which the nuclear chromatin shows increased clumping and the cytoplasm begins to acquire hemoglobin and takes on an acidophilic tint; and the orthochromatic erythroblast., the final stage before nuclear loss, in which the nucleus is small and ultimately becomes a blue-black, homogeneous, structureless mass.

Hematopoietic stem cells (HSCs): These cells give rise to all the other blood cells and are derived from the red bone marrow. They give rise to the myeloid (monocytes and macrophages, neutrophils, basophils, eosinophils, erythrocytes, megakaryocytes/platelets, dendritic cells), and lymphoid lineages (T-cells, B-cells, NK-cells). HSCs can replenish all blood cell types (i.e., are multipotent) and self-renew. A small number of HSCs can expand to generate a very large number of daughter HSCs. Phenotypic markers for mouse and human HSCs are given below.
Mouse HSC: CD34^{lo/-}, SCA-1⁺, Thy1.1^{+/lo}, CD38⁺, C-kit⁺, lin⁻
Human HSC: CD34⁺, CD59⁺, Thy1/CD90⁺, CD38^{lo/-}, C-kit/CD117⁺, lin⁻

Induced pluripotent stem cells (IPSCs): Somatic (adult) cells reprogrammed to enter an embryonic stem cell-like state by being forced to express factors important for maintaining the “stemness” of embryonic stem cells (ESCs). Mouse iPSCs demonstrate characteristics of pluripotent stem cells, including the expression of stem cell markers, the formation of tumors containing cells from all three germ layers, and the ability to contribute to many different tissues when injected into mouse embryos at a very early stage in development. Human iPSCs also express stem cell markers and are capable of generating cells characteristic of all three germ layers.

### Abbreviations

PS: phosphatidylserine

### TSP: Thrombospondin

PSSSRBCs: PS exposing sickle cells
PS47SSRBCs: PS exposing CD47 deficient sickle cells
47SSRBC: CD47 deficient sickle cells
NTTM cells: Normal cells, primary tumor cells of the same histologic type and treatment resistant or metastatic tumor cells of the same histologic type as said normal cells and said primary tumor of origin.
T47EPS cells: CD47 deficient erythroid precursors, progenitors, HSCs or iPSCs transduced with DNA extracted from primary tumor cells or normal cells of the same histologic type or treatment resistant tumor cells or metastatic tumor cells of the same histologic type as the primary tumor of origin. Such cells may be autologous or allogeneic to the host.

### DETAILED DESCRIPTION

### Sickle Cells Expressing Phophatidylserine and/or Fas and Containing SS Heme Induce a Tumoricidal Response

Sickle cells expressing PS bind to PS receptor(s) and matrix thrombospondin expressed on tumor endothelium. PS refers to the anionic phospholipid present exclusively in the inner leafet of the plasma membrane of a SSRBC and is extenalized following cell activation with both physiological and pathologic stimuli such apoptosis. The present invention contemplates that SSRBCs enriched for expression of PS and/or Fas are useful for targeting tumor endothelium as a mean of treating cancer. Synapse of SSRBCs exposing PS with cognate tumor endothelial ligands thrombospondin/αvβ3, Fas ligand (FasL) or PS receptors leads to adhesion, ROS generation and endothelial cell apoptosis. Indeed, the engagement of SSRBCs Fas receptors with FasL overexpressed on tumor endothelium results in SSRBC caspase activation and phosphatidylserine externalization. Under such conditions, SSRBCs rupture releasing SSRBC-derived heme, hemichrome and heme-nitrosyl complexes which induce tumor endothelial cell apoptosis and consequent tumor cell death.

Between 2-12% of circulating erythrocytes in patients with homozygous sickle cell anemia express PS. Such cells bind avidly to human endothelial cells. PS is normally found on the inner leaflet of the plasma membrane in healthy cells with very little exposure on the outer leaflet of most healthy cells. PS exposure on the outer leaflet serves as a signal for phagocytic recognition and removal of apoptotic cells. When the cells are induced to undergo apoptosis, they undergo a dramatic change in the amounts of PS exposed on the outer leaflet. During apoptosis, at least a fraction of the exposed PS may be oxidized and some PS recognition mechanisms (such as CD36 and MFG-E8) may preferably bind to oxidized PS.

PS-exposing red cells are found among the very high and very low density fractions of sickle cells have been reported to share the feature of loss of APLT activity. Indeed, there is a positive correlation in SCD between the levels of percent PS-positivity and red-cell—endothelial adhesion. Cell-surface PS therefore functions as an adhesion ligand. Notably normal human erythrocytes with externalized PS produced by the action of ionophore also adhere to vascular endothelium and the endothelial matrix protein thrombospondin (TSP). PS-positive red cells are prepared by treating control erythrocytes with A23187 which routinely yields a red-cell preparation with PS positivity in the range of 60% to 75%.

While minimally expressed on the cell surface under basal conditions, surface expression of the receptor for PS is up-regulated following cell activation by a variety of agonists commonly expressed in the tumor microenvironment such as cytokines IL-1, TNF-α, heme, and hypoxia (Phelan, et al., J Lab Clin Med 132:519-529 (1998)). In sickle cell anemia and thalassemia, adherence of PS exposing SSRBCs to matrix TSP contributes significantly to the vascular damage. PS-positive erythrocytes bind to TSP via its heparin-binding domain. The interaction between PS and the subendothelial TSP preferentially occurs in areas where the normal confluent cell monolayer is disrupted.

TSP is a homotrimeric 450-kDa protein, multifunctional and a matricellular glycoprotein. It is synthesized and released by endothelial cells and incorporated into their matrix which becomes exposed after endothelial injury or cell retraction induced by agonists such as thrombin. Each TSP subunit contains five distinct cell-binding domains, which interact with select protein(s), adhesion marker(s), or receptor(s). The heparin-binding domain, which is located at the amino-terminus of the polypeptide, interacts with heparin, heparin sulfate proteoglycans, sulfatides, and α1-integrins. PS-positive erythrocytes bind to both soluble and immobilized TSP via its heparin-binding domain. TSP facilitates adhesion of PS-positive red cells to the subendothelium via its αvβ3 binding domain (Brittain et al., Blood 97:2159-2164 (2005)). In its soluble form, TSP binds to PS exposing SSRBCs and interacts with adhesion receptors such as CD36, CD47, αvβ1, αvβ3, and αIIbv3. TSP also promotes formation of heterocellular aggregates by facilitating adhesive interactions with platelets and monocytes.

PS receptors on phagocytes and possibly endothelial cells that are potentially capable of recognizing PS are reviewed in Hochreiter-Hufford et al., Cold Spring Harb Perspect Biol 2013; 5:a008748 P and Zhou Z. Dev Cell 13: 758-760 (2007) incorporated by reference with their references. Such surface phagocytic phosphatidylserine receptors, include BAI1, Tim1, Tim4 and Stab2, or scavenger receptors, such as SR-A1, SR-B1, LOX1, CD68 and CD36. Additional phagocytic receptor systems require the participation of soluble bridging proteins which function as opsonins. Examples include vitronectin receptor (αvβ3 integrin) with the bridging proteins TSP1 or MFG-E8, the Mer tyrosine kinase family (Mer, Tyro3 and Ax1) with the bridging proteins Gas6 or protein S (ProtS), the low density lipoprotein related proteins LRP2 and LRP8 with the bridging protein 2GPI, and the members of the collectin or complement receptor family (e.g. calreticulin (CD91)) with the corresponding complement and collectin proteins (C1q, C3, MBL2, SP-A and SP-D). The soluble protein MFG-E8 binds to PS on apoptotic cells with high affinity whereas a second region of MFG-E8 simultaneously engages integrin αvβ3 on phagocytes. Thus, MFG-E8 through its bridging function mediates PS-dependent uptake of apoptotic cells. Two other bridging molecules, Gas6 and Protein S also recognize PS exposed on apoptotic cells; they are in turn recognized by Tyro-3-Ax1-Mer family of receptors (denoted as TAM receptors) on phagocytes. In addition to the above receptors and bridging proteins, the membrane proteins CD36 and CD68 and the soluble thrombospondins (in turn binding to membrane receptors) are capable of engaging PS.

During SSRBC senescence externalization of PS is accompanied by Fas expression. The latter is displayed at most stages throughout the maturation of the human erythroid cell. Importantly, in the mature SSRBC Fas-dependent signaling processes regulate PS externalization by downregulating APLT in a caspase-dependent manner. In addition to SSRBC aging, oxidative stress leads to stimulation of caspase 3 and caspase 3-mediated degradation of band 3. In senescent SSRBCs, Fas, FasL, FADD, and caspase 8 localize to membrane microdomains of aged and oxidatively stressed RBCs. Concomitant with formation of the Fas-associated complex, aged or oxidatively stressed SSRBCs show caspase 8 and caspase 3 activity, as well as reduced aminophospholipid translocase (APLT) activity compared to young SSRBCs. Such signaling pathways, culminate in activation of the prototypical executioner caspase 3 and PS externalization. The latter is central event linked to erythrophagocytosis.

FasL is specifically expressed by the vasculature of human solid tumors and is upregulated by the cooperative action of proangiogenic and immunosuppressive paracrine factors in the tumor microenvironment (Motz et al., Nature Med 20:607-612 (2014). Tumor-derived vascular endothelial growth factor A (VEGF-A), interleukin 10 (IL-10) and prostaglandin E2 (PGE2) cooperatively induce FasL expression in endothelial cells. Indeed, in ovarian tumors, endothelial FasL expression in tumor islets and the surrounding stroma is significantly higher compared to the endothelium in normal islets and stroma.

The present invention contemplates that SSRBCs exposing PS and expressing Fas bind avidly to PS and Fas receptors respectively expressed on the tumor endothelium leading to a potent tumoricidal response. The present invention also contemplates that SSRBC-derived heme is an important tumoricidal component of the PS/Fas expressing SSRBCs. In the tumor microvasculature, SSRBCs adhere to the vessel wall via interaction between PS/Fas and cognate receptors on the tumor endothelium. Thereupon, the SSRBCs undergo spontaneous autolysis leading to the release of extracellular ferro-hemoglobin. The latter is oxidized to ferri-hemoglobin (methemoglobin) which in turn releases free heme. The latter's highly lipophilic property enables it to lodge initially within the hydrophobic interstices of the phospholipid bilayer of cells wherein it catalyzes the oxidation of cell membrane lipid, denatures proteins and perturbs the integrity of the cytoskeleton. Heme also oxidatively denatures DNA and activates cell-damaging enzymes such as caspases and cathepsins and impairs the activity of cytosolic enzymes, including glucose-6-phosphate dehydrogenase and glutathione reductase. Heme released from lysed SSRBCs promotes endothelial cell adhesion molecule expression, increasing SS cell and PMN adhesion. Heme iron-induced oxidative stress in endothelial cells activates redox-sensitive transcription factors such as Nf-kB and activator protein-1. These transcription factors in turn induce the expression of E-selectin, VCAM-1, and ICAM-1 and the recruitment of adherent leukocytes in venules. Hemoglobin S from subjects with homozygous sickle cell anemia exhibits an enhanced rate of auto-oxidation compared with normal hemoglobin. Indeed, increased levels of superoxide and hydroxyl radical are present in sickled RBCs. Sickle erythrocyte membranes exhibit greatly enhance superoxide/peroxide-driven hydroxyl radical generation relative to normal erythrocyte membranes. This is also attributed to excessive amounts of hemichrome bound to sickle erythrocyte membranes which facilitates hydroxyl radical production after contact with endothelial cell membranes. This acquired oxidant function of SS cell membranes coupled with SS heme release from autohemolysed SSRBCs results in endothelial exposure to high levels of reactive oxygen species and significant injury leading to a tumoricidal response.

Mature SSRBCs or their progenitors expressing PS and/or Fas and heme may also contain tumor cytotoxins expressed from transgenes that are capable of inducing a tumoricidal response. SSRBCs transduced with tumoricidal cargo which includes but is not limited to superantiigens, superantigen homologues, granzyme A, B and perforin and siRNAs and miRNAs disclosed in U.S. patent application Ser. No. 14/222,292 filed on Mar. 28, 2014 are useful in this invention. U.S. patent application Ser. No. 14/222,292 (both patent applications are incorporated herein by reference in their entirety along with their references).

Such SSRBCs exposing PS are also be derived from SS precursors, progenitors, HSCs or induced pluripotent stem cells (iPS cells) obtained from sickle cell patients by transduction of these with nucleic acids encoding phophatidylserine transferase which converts CDP diacylglycerol to phosphatidylserine. The methods of isolation and preparation of SS precursor, progenitor, HSCs and iPSCs, their transduction with various vectors (including the lentiviral β globin vector) incorporating tumoricidal transgenes and their differentiation into mature SSRBCs expressing tumoricidal proteins or proteins that promote a tumoricidal response are provided in U.S. patent application Ser. No. 14/222,292 filed on Mar. 21, 2014 which is incorporated by reference in entirety with its references.

These constitutive or inducible PS expressing SSRBCs or thalassemic RBCs or normal erythrocytes are used to effectively treat murine and human tumors as described in Section on “Tumor Models” and in human clinical trials provided in Example 1.

### PS Exposing SSRBCs (PSSSRBCs) Induce a Tumoricidal Response in Mice

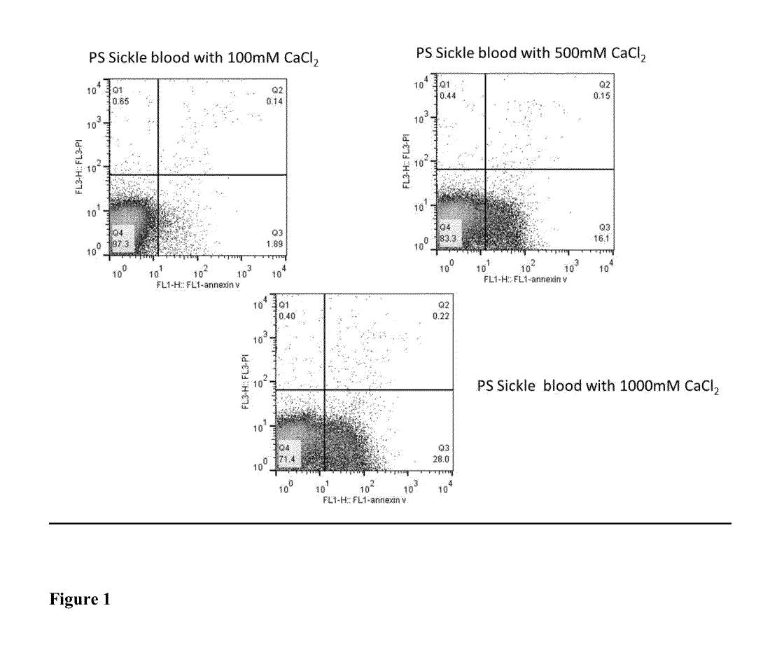

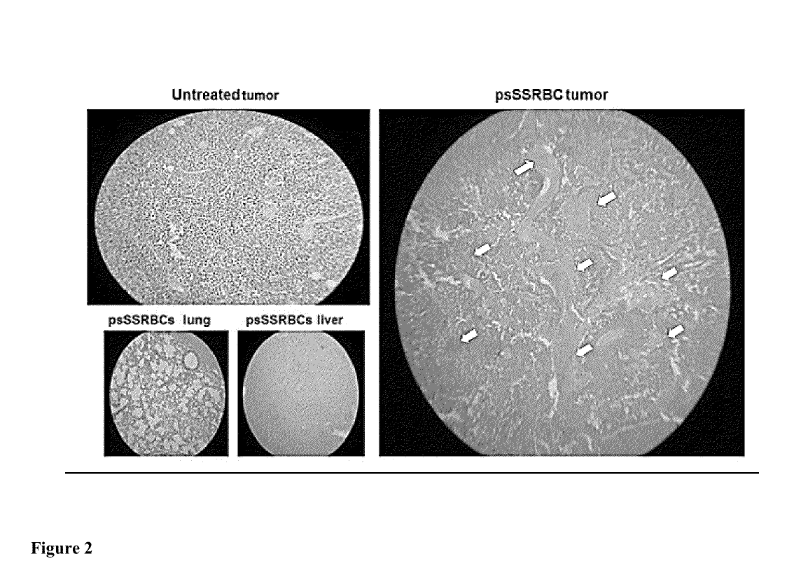

To prepare an enriched population of PS exposing SSRBCs, whole blood was obtained from sickle cell knockin mice (Townes SS model). The SSRBC population was isolated and exposed to a CaCl₂ 100-1000 mM solution for 20 minutes during which the PS exposing population of SSRBCs expanded from <1% to 28% (FIG. 1). B10F16 melanoma cells (10⁵) were implanted in the right flank of 12 week old C57BL/6 mice. When the tumors reached a diameter of 0.75 mm (at day 6) they were used for the following experiment. On days 6 and 8 after tumor implant, SSRBCs enriched in PS exposing SSRBCs (200 μL) were infused intravenously into mice with established B10F16 melanoma. Two days later tumors, lungs and liver were excised and examined histologically for evidence of necrosis, thrombosis and hemorrhage. Results are shown in FIG. 2. The tumors treated with enriched PS SSRBCs showed extensive areas of acute necrosis and focal hemorrhages over 40% of the tumor area. At higher power, several tumor blood vessels showed endothelial disruption and spillage of SSRBCs into the surrounding parenchyma. In contrast, sections of tumors from mice treated with normal RBCs similarly treated with PS and the untreated control showed relatively few scattered islands of necrosis unattended by hemorrhage. Sections of liver, lungs and heart showed no evidence of acute necrosis or hemorrhage.

Microparticles Shed from SSRBCs Expressing Phophatidylserine and/or FAS and Heme

The present invention contemplates the use of microparticles shed from SSRBCs or thalassemia RBCs which contain PS alone or PS and heme that are capable of binding to tumor endothelium generating ROS and inducing tumor endothelial apoptosis leading to a tumoricidal response. Microparticle (MP) denotes a plasma membrane vesicle shed from apoptotic, senescent or activated cell (Boulanger et al., Arterioscler Thromb Vasc Biol. 31:2-3 (2011); Camus et al., Blood 120: 5050-5058 (2012)). The size of microparticles derived from sickle cells or thalassemic RBCs ranges from 0.05 pm to 1 pm in diameter. Typically, such microparticles express surface markers that are the same as the parent cells and invariably contain SS heme or SS hemoglobin along with PS. Steady state SS patients display circulating levels of PS⁺ MPs that are 3 to 6 fold higher than in healthy adults. Priming endothelium with wild type erythrocyte MPs has minimal effects on wild type erythrocyte adhesion, and modest effects on erythrocyte adhesion of SS mice However, endothelial priming with SS erythrocyte MPs increases SSRBC adhesion about 3 fold. Likewise, fresh wild type erythrocyte MPs barely increase endothelial ROS production in culture, but SS erythrocyte MPs double endothelial ROS generation within 2 hours. In the long term, SS erythrocyte MPs incubated with endothelial monolayers for 16 hours induced significant apoptosis, whereas wild type MPs did not.

These constitutive or inducible PS expressing MPs shed from sickle cells are used in effects amounts to treat murine tumors as described in Section on “Tumor Models” and in human clinical trials provided in Example 3.

Liposomes Expressing Phophatidylserine and/or FAS and Heme

Liposomes made with PS and loaded with heme, are particularly effective in inducing adhesion, generating radical oxygen species resulting in severe injury to the tumor endothelium. PS-heme or PS-hemoglobin-laden liposomes induce enhanced linkage of SSRBCs to each other. Intravascular injection of purified PS-heme-loaded liposomes to transgenic mice with SS disease triggers SSRBC adhesion, aggregation and vaso-occlusion in tumor microvessels. Endothelial incubation with these MPs increases the adhesion of erythrocytes. At rest, SS erythrocytes are twice as adherent as wild type erythrocytes. Priming the endothelium with PS-heme liposomes for 2 hours results in a 3 fold rise in SSRBC adhesion compared to minimal adhesion by normal erythrocytes. PS-heme liposomes thus synergize with SSRBCs erythrocytes to maximize their adhesion to tumor endothelium. These therapeutic liposomes are used to effectively treat murine tumors as described in section on “Tumor Models” and in human clinical trials provided in Example 4.

### Cells of the Invention

Sickle erythrocytes or nucleated erythrocyte precursor/progenitor expressing homozygous sickle SS or hemizygous sickle S and A hemoglobin, sickle hemoglobin-C disease, sickle beta plus thalassemia, sickle hemoglobin-D disease, sickle hemoglobin-E disease, homozygous C or C-thalassemia, hemoglobin-C beta plus thalassemia, homozygous E or E-thalassemia; erythrocytes from patients with any form of sickle hemoglobinopathy such as Antilles or Trinidad that also express PS and/or Fas capable of binding to tumor vasculature are useful in this invention. PS containing MPs shed from these cells are also useful in this invention. SS erythrocytes and precursors/progenitors with at least one sickle allele are preferred It is recognized that in addition to PS-exposing SSRBCs, non-PS expressing SSRBCs may also be converted to PS exposing SSRBCs by treatment with thrombospondin TSP or TSP fragments or calcium ionophore. PS exposing SSRBCs or their precursor/progenitor cells expressing at least one S allele are useful in the treatment of cancer as described in the Tumor Models section and clinically as disclosed in Examples 1 and 2.

Therapeutically useful sickle erythrocytes are obtained from patients with sickle cell anemia, sickle cell trait and sickle cell variants

For SS erythrocytes, sickling begins at P02 40-50 mmHg and is greatest when PO2<20. This occurs in organs with sluggish circulation, high oxygen extraction, localized hypoxia and low pH such as the renal medulla and spleen and bone marrow. The likelihood of erythrocyte sickling by hemoglobin SS variants is related to amount of Hgb S present, e.g., Hgb S: 70-98%. Hgb SA: 10-40%, HgbSC: 50-60%.

Sickle trait (SA hemoglobin) affects approximately 8% of the black population in the United States or approximately 2.7 million individuals. The incidence is higher in tropical Africa and approaches 40% in some regions. Patients with sickle trait are heterozygous for the sickle cell hemoglobin gene, and less than 50% of the hemoglobin in each cell is hemoglobin-S. Polymerization of deoxy-hemoglobin in erythrocytes from patients with SA hemoglobin can occur under certain conditions and transform silent sickle cell trait into a syndrome resembling sickle cell disease with vaso-occlusion. In particular, sustained exercise and high altitude conditions cause tissue hypoxia, acidosis, dehydration, hyperosmolality, hypothermia can causing splenic infarction, exertional heat illness (exertional rhabdomyolysis, heat stroke, or renal failure) or idiopathic sudden death. Because of their proclivity to sickle and aggregate in hypoxic tissues, erythrocytes with SA hemoglobin are useful in the present invention. Milosevic et al., Gynecologic Oncology 83, 428-431 (2001)) showed that erythrocytes from patients with sickle trait may sickle in the microvasculature of solid tumors and contribute to reduced blood flow and the development of hypoxia. Hypoxia is a strong independent prognostic factor in patients with cervix cancer. While this reference did not disclose the use of erythrocytes from patients with sickle cell trait for therapy of cancer, the skilled scientist would recognize that such cells can collect and aggregate under the hypoxic conditions within tumors in a fashion similar to homozygous SS sickle cell anemia. Similarly, under hypoxic conditions hemoglobins in erythrocytes from patients with other SS variants such hemoglobin SC, hemoglobin Antilles are known to polymerize leading to sickling and aggregation. Thus this population of cells is also considered to be useful therapeutically and may be safe for transfusion since they do not sickle only under hypoxic conditions such as those encountered in tumors and not under normal physiologic conditions.

Erythrocytes with SC hemoglobin: The coinheritance of HbS and HbSC results in a clinically significant sickling disorder similar to that of sickle cell disease (HbSS). HbSC disease is usually considered less severe than Hb 20 SS disease however, some individuals manifest a condition equal in severity. HbSC disease exhibits combined symptomatology of both Hb S and Hb C diseases independently. Like SS disease, SC erythrocytes sickle under hypoxic conditions causing vaso-occlusion in ischemic tissues resulting in stroke, acute chest syndrome (chest pain, fever, dyspnea, and hypoxia), joint necrosis (especially head of femur and humerus), pain crises, acute and chronic organ dysfunction/failure, retinal hemorrhages, and increased risk of infection. Because SC erythrocytes sickle under ischemic conditions, they too are excellent candidates for use in the instant invention.

Erythrocytes with Hemoglobin S Antilles: Hemoglobin S Antilles show two mutations in hemoglobin S gene. The expected mutation of glutamic acid to valine at position 6-6 similar to hemoglobin S is accompanied by a second 30 substitution at position 6-23 of valine to isoleucine. Since the mutation at 6-23 produced no chance in the charge of the hemoglobin, it separated identically to hemoglobin S by standard techniques. Hemoglobin S Antilles is much less soluble than hemoglobin S. The consequence is that people heterozygous for hemoglobin A and hemoglobin S Antilles have symptoms and complications similar to those of patients with homozygous sickle cell disease. Because Hgb S Antilles erythrocytes sickle under hypoxic conditions, these cells are also excellent candidates for use in the present invention.

The hemoglobin S gene therefore is a critical component of the instant invention. The cells bearing the hemoglobin S gene signature home to hypoxic niches within tumors where they form microaggregates and occlude tumor microvessels. Under such hypoxic conditions, hemoglobin S polymerizes leading to membrane disruption and autohemolysis. SSRBCs but not normal RBCs have shown an ability to induce tumoricidal effects versus established breast tumors and melanoma. Likewise hemoglobin S/C or S/thalassemia polymerizes under deoxygenating conditions leading to microaggregate formation and microvessel occlusion. Sickle cell trait cells have been shown by Milosevic et al., Gyn Oncol 83: 428-431 (2001) to localize in the microvessels of cervical carcinomas. Thus cells with at least one S allele are capable of homing and aggregating in hypoxic tumor microvessels. The skilled scientist would therefore readily believe that cells with at least one hemoglobin S allele are able to target hypoxic tumors, form microaggregates, obstruct tumor blood flow and induce a tumoricidal effect.

### Methods

### Isolation and Enrichment PS Exposing SSRBCs

SSRBCs are obtained from patients with sickle cell anemia and collected in heparin or EDTA after informed consent is obtained. SSRBCs are pelleted by centrifugation, washed twice with 0.9% NaCl and once with incubation buffer, and finally diluted in incubation buffer to the appropriate hematocrit. Either Hanks' buffered salt solution, pH 7.4 (HBSS; Sigma, St Louis, Mo.), or 10 Mmol/L Tris/HCl buffered saline, pH 7.4 (TBS), is used as buffer throughout the experiments; Similar results are found with both buffers. Additional reagents such as CaCl₂ and N-ethyl maleimide (NEM), are added as indicated below. Mouse erythrocytes are obtained through retro-orbital sinus puncture, are collected on citrated tubes and mixed with anticoagulant heparin buffer (5 UI/mL).

### Calcium Ionophore Treatment of SSRBCs.

Calcium and ionophore treatment induces membrane lipid scrambling. SSRBCs at a 16% hematocrit are equilibrated in incubation buffer with 1 mmol/L calcium for 3 minutes at 37° C. Subsequently, calcium ionophore A23187 is added to the RBC suspension to a final concentration of 4 pmol/L. This final mixture is incubated for 1 hour. The process is stopped by a wash with 2.5 mmol/L EDTA to remove calcium. Subsequently, the cells are washed three times in buffer containing 1% bovine serum albumin (BSA) to remove the ionophore and are resuspended in buffer without BSA to prevent calcium uptake during annexin V-labeling of the cells (see below). This removal of ionophore is crucial, as omission and subsequent incubation in the 1.2 mmol/L calcium buffer used for annexin V labeling leads to massive hemolysis.

Expansion of PS Exposing SSRBCs by Ligation of CD47 with TSP, TSP-1-Derived 41NK Peptide or Anti-CD47.

Ligation of CD47 with TSP-1-derived 41NK peptide, anti-CD47mAb and TSP-1 induces PS expression on SSRBCs. CD47 is a 47-52 kDa transmembrane glycoprotein with a ubiquitous expression profile in human tissues that includes erythrocytes. Ligation of CD47 is provided by monoclonal antibodies (mAbs) that recognized their natural ligand thrombospondin-1 (TSP-1) or the specific CD47-binding peptide 41NK, derived from TSP-1. Erythrocytes derived from patients with sickle cell anemia, thalassemia are cultured at a density of 4×10⁶/ml for 24 h with the indicated treatments. The level of PS expression measured by Annexin V binding increases nearly 12 fold. The mean percentage of Annexin V positive erythrocytes using flow cytometry following treatment with 20, 50 and 100 μg/ml 41NK or the control 41NG peptide increases nearly 12 fold. The mean percentage of Annexin V positive erythrocytes following treatment with anti-CD47mAb (10 μg/ml) and TSP-1 (100 μg/ml) increases >59% and >30% respectively (Head et al., Br J Haematol 130:788-790 (2005)). These expanded PS expressing SSRBCs are highly useful in the instant invention in inducing tumor endothelial injury and a tumoricidal response.

### Magnetic Cell Separation.

Magnetic beads (average size, 15 nm) covered with an anti-FITC antibody are supplied by Miltenyi Biotec Inc (Auburn, Calif.). The stock solution of beads is fivefold diluted in annexin V labeling buffer. SSRBCs labeled with FITC annexin V are washed, and 6×10⁷ cells are added to 80 μL of buffer. To the cell suspension, 20 μL of the diluted beads is added. After a 10-minute incubation at room temperature, the cells are separated in a magnetic separation setup (Minimac; Miltenyi Biotec Inc) according to the standard protocol supplied by the manufacturer.

Identification of SSRBCs with Exposed PS by Flow Cytometry

Samples are analyzed on a Becton Dickinson FACScan flow cytometer (Becton Dickinson, San Jose, Calif.). Acquisition and data analysis are performed using LYSYS 11 software (Becton Dickinson). Ten thousand events per sample are acquired to ensure adequate mean fluorescence levels. The light scatter and fluorescence channels are set at a logarithmic gain. The forward angle light scatter setting is E-1 with a threshold of 36. The red cell population is defined by size in forward and side scatter plots. Events that correlate with intact RBCs are analyzed for fluorescence intensity. Mean fluorescence intensities are expressed in linear mode, and positive fluorescence is defined by comparison with unlabeled control samples. The treatment of SSRBCs with NEM followed by incubation with

Calcium and ionophore slightly change the forward scatter characteristics of the population in the flow cytometer as compared with control RBCs (not shown). Gated regions are adjusted accordingly. This shift in characteristics can be expected because this treatment will result in a number of changes in the cell, including alterations of morphology and loss of membrane material by vesiculation.

Incubation of SSRBCs with fluorescently labeled anti-glycophorin A antibody (Becton Dickinson) shows a clear labeling of the population. Treatment of SSRBCs with NEM followed by incubation with calcium and ionophore resulted in the binding of FITC-labeled annexin V. The population of cells significantly labeled with annexin V above background is indicated by marker MI. For example, 98% of the cells meet this criterion.

Isolation of Shed Microparticles from SSRBCs Expressing PS

Standard methods for isolating microparticles are well known in the art (Mause et al., Circ Res; 107:1047-1057 (2010); Camus et al., Blood 120: 5050-5058 (2012); Marva et al., Blood 83: 242-249 (1994); Farru et al., Haematologica 99, 571-575 (2014)). Annexin V binds to externalized phosphatidylserine in a calcium dependent manner and thus labels microparticles expressing phosphatidylserine. Fluorescence activated cell sorting (FACS) is used to separate the microparticles in the blood sample. Magnetic beads labelled with monoclonal specific antibodies are used for the positive selection of microparticles. Alternatively microparticles are isolated from a blood sample using size exclusion columns or filters to purify microparticles of specific sizes. In filtration from plasma a 0.1 pm pore filter is commonly used.

Ultracentrifugation is also performed, before filtration. To trigger shedding of MPs mouse or human erythrocytes are suspended in polyvinylpyrrolidone (31 mPa·s) and placed in a rotating type LORCA ektacytometer (R&R Mechatronics, Hoorn, Holland) in the absence or presence of TSP1 (25 μg/ml), or TSP1-derived peptides 4N1-1 or 4N1-2 (25 μM). Shear is applied at 1500 s-1; 2.5 minutes. SSRBCs are immediately separated and supernatant PS+ MP quantified by FACS. Concentrated MP stocks are generated from 10 to 20 ml of supernatant from SSRBCs suspended in RPMI-1640 (4.106/ml) treated with carboxyterminal peptide 4N1-1. Suspensions are centrifuged at 400 g for 15 min to discard cells, and ultracentrifuged at 20500 g for 45 min to pellet MP, which are resuspended in filtered (0.2 pm) DMEM and stored at −80° C. MP concentrations are determined by labeling with 2 pi FITC-conjugated annexin-V (Roche Diagnostics, France) diluted in 100 pi reaction buffer with 5 mM CaCl₂. CaCl₂ is omitted in negative controls. MP are analyzed on a Coulter EPICS XL flow cytometer (Beckman Coulter) and MPs are identified in forward light scatter (FSc) and side-angle light scatter (SSc) intensity dot plots set at logarithmic gain, as events of 0.1-1 pm in diameter. MP-size events are analyzed in FL/FSC fluorescence dot plots to determine annexin-V labeling. 4N1-1 also triggers a 40 fold rise in SSRBCs. SSRBCs shed about twice as many MP than healthy erythrocytes. Quantification of ‘free’ heme in the shed MPs is carried out by spectrophotometry at 540 nm or 575 nm, by the Drabkin method, by the Kahn method or the AI method or the AIII method.

### MP Characterization

Erythrocytes are suspended in polyvinylpyrrolidone (31 mPa/s) and placed in a rotating type LORCA ektacytometer (R&R Mechatronics), in the absence or presence of TSP1 (25 g/mL), or TSP1-derived peptides 4N1-1 or 4N1-2 (25M). Shear is applied at 1500 seconds 1; 2.5 minutes. Supernatant PS expressing MPs are labeled with FITC-conjugated annexin-V (Roche Diagnostics) diluted in reaction buffer with 5 mM CaCl₂ and quantified on an EPICS XL flow cytometer (Beckman Coulter). MPs are identified in forward light scatter (FSc) and side-angle light scatter (SSc) as events of 0.1 to 1 m in diameter. MP-size events are analyzed with respect to calibrated fluorescent microbeads (Flowcount). PS MPs prepared in this fashion and by the inducible techniques described invariably contain heme derived from the erythrocyte of origin. Such heme or hemoglobin is a significant component of the PS MPs since after binding to the tumor endotheliuma heme contributes to the tumor endothelial injury and the tumoridical response. Measurement of heme in such MPs is carried out as described below.

Liposomes Expressing Phophatidylserine and/or FAS and Heme

Multi- or unilamellae liposome containing PS and heme are prepared by previously described methods (Cullis et al., Adv Drug Deliv Rev, 3: 267-282 (1989); Wickramaratne et al., Hemoglobin, 29:27-42 (2005); Marva et al., Blood 83: 242-249 (1994)). Liposomes from each of two types of phospholipid: dioleoyl (18:1) phosphatidylserine (dPS) or bovine brain phosphatidylserine (bps) (Avanti Polar Lipids, Alabaster. Ala.) are prepared. Two milliliters of the lipid dissolved in chloroform (10 mg/mL) is evaporated under nitrogen and desiccated overnight. Assay buffer (1.5 mL) is added to the lipids and large (100 nm) unilamellar liposomes are made by repeated extrusion through polycarbonate filters (Avestin Inc, Ottawa, Canada). The liposome size is confirmed (97.3±29.0 nm) by quasielastic light scattering. The pH of the final liposomes in assay buffer is adjusted to pH6.5, and then the phospholipid content is determined by measurement of lipid phosphorus. The preparation of liposomes containing chemotherapeutic agents is recently reviewed by Torchilin D. Nature Rev 4: 145 (2005)) and incorporated by reference.

### PEG Liposomes

Polyethyleneglycol-2000 (PEG)-liposomes containing distearoylphosphatidyl-ethanolamine (PEG-liposomes) are prepared as follows. Liposomes are first sized by multiple extrusion through pairs of stacked polycarbonate membranes using a medium pressure extruder (Lipex Biomembranes, Vancouver, BC, Canada). Averager phospholipid recovery after liposome preparation is 80%. Particle size distribution is determined by dynamic light scattering with a Malvern 2000 system equipped with a 25-mW neon laser (Malvern Instruments. Malvern, United Kingdom). Mean size of the small PEG-liposomes is 80 to 85 nm with a polydispersity index less than 0.1. The liposomes are administered intravenously at a dose of 5 μM/kg phospholipids in a volume of 0.1 ml.

### HB/Liposome Reaction Mixture

To 1,000 μL of Hb, 100 μL of liposomes are added to achieve final concentrations of 6.8 μmol/L heme and 0.25 mg/mL lipid in assay buffer (5 mmol/L KCl, 10 mmol/L Tris, pH 6.5). In some experiments, conditions of higher pH (7.2) and/or higher salt concentration (100 mmol/L KCl) are used. All incubations are performed at room temperature. The following parameters are monitored.

### Production and Isolation of Heme

Heme is prepared from SSRBCs or thalassemic RBCs by following method disclosed in U.S. Pat. No. 4,431,581 which is incorporated by reference. Briefly, a heme concentrate containing pure heme in readily absorbable form is obtained from cleavage of hemoglobin from whole human blood or SSRBCs. This mixture is treated with a dehydrating agent, preferably a lower alcohol or a mixture of alcohols, either at a pH of at least 8.0 or in the presence of a substance promoting the separation, preferably imidazole or an imidazole derivative, at a pH of at least 6.0, preferably 6.5 to 8.5. The solid blood substance is separated and the heme concentrate is recovered from the remaining solution. The raw material comprises, for example, whole blood, red blood cells, or hemoglobin in another milieu. A particularly suitable starting material is the heme product obtained according to the method described in Swedish Patent No. 7407882-5 and in the Swedish Patent Application No. 7513987-3 both of which are incorporated in entirety by reference. According to this method, the hemoglobin is cleaved in an aqueous ethanol solution with an ethanol content of at least 40 percent by volume, at a pH of less than 4.5. The precipitate of heme product formed is separated. The remaining globin solution is practically free of heme. This known product contains about 10 percent heme by weight. A heme concentrate has a heme content of about 40 percent by weight. The heme concentrate is separated from any other blood substance, i.e. mainly protein, so that the heme concentrate is converted into the dissolved form while the remaining substance is present in undissolved form. Suitable extraction agents are ethanol, methanol, propanol, isopropanol, butanol, isobutanol, ethylene glycol, and glycerol. The content of extraction agent must be over 40 percent by weight and pH value must, be at least 6.0. To prevent denaturation of the protein the temperature is maintained at preferably less than −5° C.

To prevent dissolution of blood substance from the heme/blood substance mixture simultaneously with the heme concentrate in the extraction solution, the blood substance, mainly globin is precipitated before the extraction. The precipitation is effected, for example, by adjusting the pH for the mixture to 7-8, the isoelectric pH range for globin, and/or by adding various salts such as Na, K, NN 4 chloride, sulphate, citrate, phosphate, and tartrate. In addition, other agents that promote the separation are added, such as amino acid, e.g., glycine, glutamine, or lysine. Glycine, in particular, increases the yield of heme concentrate up to about 99 percent, when the other conditions at the same time are ideal. Without the addition of glycine, the yield remains at about 93 to 95 percent. The undissolved substance is separated by centrifugation or filtration. The heme concentrate is precipitated out of the remaining supernatant or the filtrate, by lowering the pH to <6.5, preferably 4.0-6.0. A black, flaky precipitate is then formed. The precipitation is quantitatively increase when the pH value is less than 5. To obtain a more quantitative precipitation the precipitation is carried out at a temperature of <0° C. The precipitate is separated by centrifugation or filtration, and washed free of ethanol, salt and imidazole with ice water. The precipitate is dissolved in water by raising the pH to 8, and the solution is freeze dried. The yield of heme is about 90 percent. Because the separation of heme concentrate is carried out at a pH<10.5 and at a temperature lower than 0° C., the protein is recovered is non-denatured whereby its functional properties are preserved. The protein is particularly satisfactory when recovered at a pH<9. The heme concentrate obtained in the method according to the invention usually has a heme content of about 35 to 40 percent.

### Measurements of Heme

Standard methods for the level of heme and hemoglobin are well known. Typically said levels may be determined by spectrophotometry. The classical method for determining the level of hemoglobin in a sample utilizes the method of Drabkin. (Drabkin D L, Austin J H J Biol Chem 112:51-58, (1935)). Briefly, potassium ferricyanide is added to the sample which results in oxidation of the heme iron to, produce cyanomethemoglobin. Cyanide ions then convert the methemoglobin to cyanomethemoglobin, a more stable chromagen. The hemoglobin concentration of the sample is then determined by measuring the absorbance of the cyanomethemoglobin at 540 nm. Another method is also described in Kahn S E, Watkins B F, Bermes E W Jr Ann Clin Lab Sci 11: 126-31 (1981); Fairbanks V F, Wiesmer S C, O'Brien P C Clin Chem 38:132-140 (1992)). Another method consists at measuring absorbance at 575 nm, the wavelength of an absorption peak specific to heme and hemoglobin. Spectrophotometric methods preferably include an approach to correct for the enhanced turbidity of plasma in sickle cell disease. Such methods include subtracting absorbance values measured at 600 nm or 650 nm.

### PS Exposing CD47 Deficient SSRBCs (PS47SSRBCs)

In sickle cell anemia patients, 2-12% of circulating SSRBCs are PS exposing compared to less than 1% of nRBCs (nRBCs) in normal individuals. Such PS-exposing cells are a hallmark of senescence resulting from a combination of ROS-induced damage and an ATP-limiting environment that impairs membrane flippase activity and allows scramblase-mediated transfer of PS from the inner to outer membrane leaflet (Kuypers et al., Cell Mol Biol 58: 147-158 (2004); Setty et al., Blood 99: 1564-1571 (2002)). Notably, up to 50% of normal RBCs lose CD47 expression with advancing age and a large percentage these cells are PS exposing. Both PS exposing and CD47 deficient RBC subpopulation exhibit shortened survival in vivo (Oldenborg et al., Science 288:2051-2054 (2000); Khandelwal et al., Transfusion 47: 1725-1732 (2007)). Notably, PS exposing SSRBC show robust adhesion to activated endothelial cells in vitro (Schroit et al., J. Biol Chem 260: 5131-5138 (1985); de Jong et al., Blood 98: 1577-1584 (2001)). The adhesive phenotype of PS exposing RBCs is upregulated significantly by ligation of surface CD47 with thrombospondin (TSP). The latter activates αvβ1 (VLA-4) whose cognate endothelial ligands are endothelial VCAM-1 and matrix TSP (Brittain et. al., J Clin Invest. 107:1555-62 (2001)). Interestingly, PS exposing RBCs also coexpress Fas (Mandal et. al., J Biol Chem. 280:39460-7 (2005)) whose cognate ligand FasL is highly overexpressed on the upregulated endothelium of multiple epithelial tumors (Motz et al., Nat Med. 20:607-15 (2014)).

The present invention contemplates that the subset of PS exposing CD47 deficient SSRBC (PS47SSRBCs) adheres avidly to tumor microvessels and induces tumor microvessel occlusion. As discussed further below, such PS47SSRBCs also activate host innate and adaptive antitumor immune responses. Importantly CD47 deficient RBCs also promote phagocytosis by splenic macrophages (Oldenborg et al., supra (2000)). Such enhanced phagocytosis by both splenic and tumor macrophages is ascribed to their recognition of the same dominant phagocytic signal, calreticulin, expressed on apoptotic CD47 deficient RBCs whose surface CD471 is ligated by specific antibodies (Shyra et al., Cell 123: 321-334 (2005)).

What is CD47 and how does it work? CD47 is a protein 50 kDa consisting of an extracellular Ig domain and five membrane-spanning segments with a small cytoplasmic tail which is overexpressed on in a variety of cancer cells. It suppresses macrophage phagocytosis by interaction with signal regulatory protein alpha (SIRPa) a heavily glycosylated transmembrane receptor on macrophages. Upon ligation with CD47, the SIRPα immunoreceptor tyrosine-based inhibitory motif (ITIM) is phosphorylated and recruits SHP phosphatases resulting in inhibition of myosin accumulation at the cell surface and thereby precluding phagocytosis (Oldenborg et al., ISRN Hematology Article ID 614619, 19 pages (2013)). Disruption of interactions between CD47 on the target cell and SIRPa, permits uptake of viable tumor cells by macrophages in a calreticulin/LRP-dependent manner (Shyra et al., supra (2005); Obeid et al. Nat Med 13(1):54-61 (2007)). Indeed, ligation of the CD47 receptor on tumor cells with specific antibodies results in activation of macrophage proinflammatory and phagocytic function resulting in a potent tumoricidal response against a broad array of malignant cells (Chao et al., Science Translational Medicine. Vol 2 Issue 63 63ra94 (2010).

Calreticulin, a peptide-binding chaperone is highly overexpressed on the vast majority of tumors (Obeid et al., supra (2007)). It is the dominant prophagocytic signal on both apoptotic tumor cells and CD47 deficient RBCs. Indeed, calreticulin on apoptotic tumor cells also elicits potent tumor specific immunity via macrophage cross presentation into the MHCI pathway and even dictates the immunogenicity of several major cancer cells (Shyra et al., supra (2005); Obeid et al., supra (2007); Basu S, et al., J Exp Med 189:797-802 (1999)). Calreticulin expressed on CD47 deficient SSRBCs is similarly recognized by the LDL receptor related protein (LRP-1) on splenic macrophages and triggers phagocytosis of apoptotic RBCs (Oldenborg et al supra (2000)). Such macrophage phagocytosis via the LRP-1 receptor occurs even in the presence of SIRPa blockade. Hence, the combination of inhibition of the CD47-SIRP-α (the “don't eat me” pair) and calreticulin overexpression promotes efficient macrophage phagocytosis of both calreticulin bearing tumor cells and apoptotic CD47 deficient RBCs. We therefore contemplate that PS47SSRBCs transfused into tumor bearing host not only shut down tumor blood flow and destroys tumor endothelium but also circumvents the CD47-SIRPa inhibition of macrophages and unleashes a systemic calreticulin-based innate and adaptive anti-tumor immune response. These PS47SSRBCs are used in effective amounts to treat murine tumors as described in Section on “Tumor Models” and in human clinical trials provided in Example 2.

### Adhesion of PS Exposing and PS47SSRBCs to Tumor Endothelium:

We contemplate that the tumor selectivity of PS exposing SSRBC and PS47SSRBCs is due to the presence of upregulated adhesion receptors expressed on these SSRBCs that bind to cognate ligands overexpressed on the activated tumor vasculature. Such ligands include αᵥβ3, VCAM-1 and matrix thrombospondin/fibronectin. In the absence of activated non-tumor endothelium in the tumor bearing host, such upregulated tumor endothelial receptors outcompete other microvessels for binding SSRBCs. The PS exposing SSRBCs also adhere to putative PS receptors and FasL expressed on tumor microvessels. Such tumor preference by PS exposing SSRBCs and PS47SSRBCs allows sufficient numbers of SS cells to target the tumor to produce an anti-tumor response.

To confer even greater tumor specificity on the PS exposing and PS47SSRBCs we introduce a tumor targeting ligand such as epidermal growth factor (EGF) or a single chain tumor specific antibody fragment using our β globin lentiviral transgene system disclosed in U.S. patent application Ser. No. 14/222,292, U.S. patent application Ser. No. 13/367,797 and U.S. patent application Ser. No. 12/586,532 which are incorporated by reference. The EGF receptor is highly overexpressed on malignancies including but not limited to breast, lung, colon, head neck, uterine, renal cell and bladder carcinomas. The EGF 53-amino acid polypeptide of known sequence contains six cysteine residues, which form three intrachain disulphide bonds. Examination of the precursor 4,706-bp nucleotide sequence reveals one translational reading frame that codes for an uninterrupted sequence of 1,168 amino acid residues. The 53 amino acids of mouse EGF are embedded within this sequence. The open reading frame that includes the EGF amino acid sequence starts with an initiation codon ATG, three nucleotides downstream from an in-frame termination codon (TAA). The initiator methionine is followed by a stretch of 29 amino acids which are hydrophobic. The sequence of the mature EGF protein begins at position 977 of the precursor and ends at amino acid 1,029 as shown in FIG. 2A of Gray et al., Nature 303: 722-725 (1983)) which is incorporated by reference in its entirety with its references. Any receptor ligand or antibody fragment whose cognate ligand/antigen is expressed or overexpressed on tumors is useful in this invention. Such tumor targeting devices can be incorporated into the PS exposing SSRBCs or PS47SCs alone or together with tumoricidal transgenes including but not limited to granzyme A, B, perforin, superantigens SEG/SEI as disclosed in U.S. patent application Ser. No. 14/222,292, U.S. patent application Ser. No. 13/367,797 and U.S. patent application Ser. No. 12/586,532 incorporated in entirety by reference with their references.

### Adhesion of PS47SSRBCs to Tumor Endothelium In Vitro and In Vivo

Human PS47SSRBCs are isolated, separated and enriched using Annexin A and magnetic beads as described (Kuypers et al., Blood 87:1179-1187 (1996)); de Jong et al., supra (2001); Khandelwal et al., supra (2007)). CD47 deficient SSRBC are prepared by knockdown of the CD47 gene in SCA+, kit+, lin⁻ bone marrow cells of SS mice (Townes model) using siRNA or shRNA as described below. They are also produced by crossing CD47 deficient mice (Jackson) with the homozygous SS mice (Townes model). Human and mouse PS47SSRBCs demonstrate robust binding to activated HUVEs under static and flow conditions deploying well established methods. Using the intravital microscopy of 4T1 mammary tumors implanted in the dorsal skin window of nude mice described in Terman et al., PLoS ONE PLoS ONE 8 (1): e52543. doi:10.1371 (2013), dil labeled PS47SCs SSRBCs infused intravenously localize rapidly, bind avidly to tumor microvessels form microaggregates and shut down tumor blood flow.

### PS47SSRBCs Induce Macrophage Activation, T Cell Priming & Anti-Tumor Responses

Splenic pulp macrophages demonstrate more than 4 fold greater phagocytosis of PS47SSRBCs relative to PS-treated CD47 deficient normal RBC controls. Such PS47SCs also induce 2-4 fold greater macrophage secretion of proinflammatory cytokines IL-12p40, TNF-α RANTES and monocyte chemotactic protein-3 (MCP-3) compared to controls. Macrophage phagocytosis of PS47SSRBCs also induces a 3 fold greater degree of CD8+ T cell priming than PS-treated CD47 deficient normal RBC controls. These CD8+ T cells primed by exposure to PS47SSRBCs are adoptively transferred to C57B1/6 mice bearing Lewis lung carcinoma and B16F10 melanoma cells and induced a tumoricidal response.

### Testing the Antitumor Effect of PSSSRBCs, PS47SSRBCs and CD47 Deficient SSRBCs in 4T1 Mammary Tumor Model

The 4T1 used in our published studies exhibits all the major features of stage 4 triple negative breast cancer with poor survival and is generally regarded as an excellent and challenging model of metastatic breast cancer (Zhang et al., Nature Cell Biol 15:184-194 (2013)). For in vivo experiments, tumor cells are harvested during exponential growth, washed and resuspended in media. Cell viability is greater than 90%. 4T1 mammary tumor cells (10⁶) in 25 μL PBS) are implanted in the right flank of NOD/SCID mice. When the tumors reach a diameter of 50 mm, 10¹⁰ PS exposing SSRBCs (PSSSRBCs) or CD47 deficient SSRBCs (47SSRBCs) or PS47SSRBCs are administered intravenously and induce a tumoricidal response.

### Tumors

A mammal with a neoplasia having any of the following neoplasias: glioblastoma, melanoma, neuroblastoma, adenocarcinoma, glioma, soft tissue sarcoma, and various carcinomas (including prostate and non-small cell lung cancer) can be effectively treated with the above compositions. Suitable tumors further include astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, squamous cell lung carcinomas, chordoma, angiosarcoma, endotheliosarcoma, squamous cell carcinoma, bronchoalveolarcarcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in recurrence.

Pharmaceutical Administration of PSSSRBCs, PS47SSRBCs, 47SSRBCs, PS-Heme Microparticles Derived from SSRBCs and PS-Heme-Liposomes

PSSSRBCs, PS47SSRBCs, 47SSRBCs, PS-heme microparticles derived from SSRBCs and PS-heme-liposomes are administered parenterally preferably intravenously by infusion or injection but also may be injected intratumorally, intrapleurally, intraperitoneal, intrathecally, intrapericardially, intravesicularly, subcutaneously, intralymphatically, intraarticularly, intradermally, intracranial, intraarticularly or intramuscularly. They may be administered in a controlled release formulation.

The pharmaceutical compositions of the present invention generally comprise an effective amount of the above compositions. The compositions are dissolved or dispersed in a pharmaceutically acceptable carrier or aqueous medium. One or more administrations may be employed, depending upon the lifetime of the drug at the tumor site and the response of the tumor to the drug. Administration may be by syringe, catheter or other convenient means allowing for introduction of a flowable composition. Parenteral dosages for PSSSRBCs or PS47SSRBCs or 47SSRBCs in mice range from 100 to 250 μL delivered intravenously (as described herein under in the Tumor Models) and in humans from 25 to 250 ml. administered intravenously by infusion or injection. Administration may be every 2-3 days, weekly, or biweekly or at monthly intervals. Parenteral doses of PS-heme microparticles derived from SSRBCs and PS-heme-liposomes are in a range of 50-250 μL for mice (in the tumor models described herein) and 10-300 ml for humans.

The phrases “pharmaceutically or pharmacologically acceptable” refer to molecular entities and compositions that do not produce an adverse, allergic or other untoward reaction when administered to an animal, or a human, as appropriate. Veterinary uses are equally included within the invention and “pharmaceutically acceptable” formulations include formulations for both clinical and/or veterinary use.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. For human administration, preparations meet sterility, pyrogenicity, general safety and purity standards as required by U.S. Food and Drug Administration. Supplementary active ingredients can also be incorporated into the compositions.

“Unit dosage” formulations are those containing a dose or sub-dose of the administered ingredient adapted for a particular timed delivery. For example, exemplary “unit dosage” Formulations are those containing a daily dose or unit or daily sub-dose or a weekly dose or unit or weekly sub-dose and the like.

The compositions of the present invention are preferably formulated for parenteral administration, e.g., introduction by injection, infusion. They may also be administered intravenously, intramuscularly, intradermally, intraperitoneally, intrapleurally, intraarticularly. Means for preparing aqueous compositions that contain these compositions are known to those of skill in the art in light of the present disclosure. Typically, such compositions can be prepared either as liquid solutions or suspensions; solid forms suitable for using to prepare solutions or suspensions upon the addition of a liquid prior to injection can also be prepared.

The techniques of preparation are generally well known in the art as exemplified by Remington's Pharmaceutical Sciences, 16th Ed. Mack Publishing Company, 1980, or most recent edition, incorporated herein by reference. Moreover, for human administration, preparations should meet sterility, pyrogenicity, general safety and purity standards as required by the U.S. Food and Drug Administration. Upon formulation, the therapeutic compositions are administered in a manner compatible with the dosage formulation and in such amount as is therapeutically effective.

Transduction CD47 Deficient SSRBC Precursor, Progenitor, HSCs or iPSCs (T47EPS) with Viral-Altered Genomic DNA from Tumor Cells or Normal Cells or Treatment Resistant Tumor Cells of the Same Histologic Type:

### Background

The ability of macrophages to engulf cells is triggered by the binding of specific receptors on macrophages to their ligands. Receptors on macrophages include phosphatidylserine receptors and low-density lipoprotein-related protein (LRP). Lipopolysaccharide and calreticulin (CRT) are typical ligands which induce a prophagocytic signal to macrophage receptors. The activation of the phosphatidylserine receptor serves as a prophagocytic signal for macrophages during apoptosis. Phagocytosis is prevented by the interaction of CD47 expressed on SSRBCs and signal regulatory protein a (SIRPA) exhibited on macrophages. Phagocytosis by macrophages of viable cells is therefore regulated by the balance of prophagocytic CRT-LRP and antiphagocytic CD47-SIRPA signaling interactions.

CD47 is a member of the Ig superfamily that is ubiquitously expressed in hematopoietic and nonhematopoietic cells. CD47 interacts with SIRPA through its IgV-like domains. SIRPA is a transmembrane protein that contains 3 Ig-like domains within the extracellular region. SIRPA is expressed in macrophages, myeloid cells, and neurons. The cytoplasmic region of SIRPA has immunoreceptor tyrosine-based inhibitory motifs. Binding cell-surface CD47 with SIRPA on macrophages provokes inhibitory signals through phosphorylation of the immunoreceptor tyrosine-based inhibitory motifs of SIRPA, activating inhibitory tyrosine phosphatases such as SHP1 and SHP2. This signaling inhibits myosin assembly of macrophages, thereby inhibiting phagocytosis. Thus CD47-SIRPA self-recognition system is primarily used in RBC clearance to maintain homeostasis of the blood and functions as a “don't eat me” signal to ensure that autologous cells are not inappropriately phagocytosed.

Lack of autonomous CD47 expression results in aggressive phagocytosis of such red blood cells by splenic macrophages of normal mice (Oldenborg et al., Science 288: 2051-2053 (2000)). Such phagocytosis leads to development of autoimmune hemolytic anemia (Oldenborg et al., Blood 99: 3500-3504 (2002)). In addition to CD47 deficiency, macrophages of C57BL/6 mice require activation of their Fc or complement receptors to efficiently phagocytose CD47^{-/-} RBCs. Thus, the present invention contemplates that besides CD47 deficiency TCD47Es also require opsonizing antibodies, complement components and thrombospondin to trigger macrophage phagocytosis. Such secondary signals polarize the macrophages into an antigen presenting mode leading to MHCI processing and presentation to T cells resulting in activation of cytotoxic T cells.

In the process of erythroid differentiation normal and CD47 deficient erythroid normoblasts undergo enucleation forming nuclear free reticulocytes and pyrenocytes which contain nuclear material. The pyrenocyte binds to macrophage VCAM-1 via its a₄β1 integrin and actively extrudes its nucleus into the macrophage by an active transport process that utilizes the MOTC, tubular rails and microvesicles (Chasis et al., Blood 112: 470-478 (2008); Keerthivasan et al., Blood 116: 3331-3340 (2010)). The enucleation process represents one of the most effective host methods of transporting whole nuclear DNA from one cell to another. Like normal erythroblasts, CD47 deficient erythroblasts extrude their nuclei into bone marrow derived macrophages situated in erythroid hematopoietic islands. Such nuclei from both normal and CD47 deficient erythroblasts contain phosphatidylserine membranes which promote their uptake by macrophages. Under normal conditions, the macrophage processes these nuclei into the apoptotic pathway. In the absence of a CD47 signal, however, macrophages aggressively phagocytose the nuclei and process these antigens into the MHC1 pathway for presentation to CD8+ T cells. Phagocytosis of CD47 deficient erythroblasts leads to the host autoimmunization against RBCs (Oldenborg et al., Blood 99: 3500-3504 (2002)), the development of an M1 macrophage phenotype and a pro-inflammatory cytokine profile. This process also results in cross processing and presentation antigens in the macrophage MHC I pathway leading to cytotoxic T cell activation (Tseng et al., Proc. Natl. Acad. Sci. 110: 11103-11108 (2013)). Hence, this invention exploits the enucleative transfer of tumor specific DNA from CD47 deficient SS precursors to macrophages to promote M1 macrophage polarization and activation of a tumor specific cytotoxic T cell response.

In support of the immunogenic nature of such CD47 deficient RBCs, CD47 deficient mice exhibit hyperactive T cell responses and autoimmunity. Likewise, in patients with hemophagocytosis lymphohistiocytosis (HPLH), CD47 deficient RBCs are aggressively phagocytosed by macrophages which process them into the MHCI pathway leading to the generation of a large numbers of CD8+ cytotoxic T cells (Kuriyama et al., Blood 120 4058-4067 (2012)). Such macrophages also secrete cytokines such as IFN-γ, TNFα, IL-6, mCSF, RANTES, IL12 and nitrous oxide.

The present invention contemplates transducing CD47 deficient erythroid precursors, progenitors, HSCs or iPSCs (collectively T47EPS cells) with DNA extracted from tumor cell or normal cells or treatment resistant tumor cells or metastatic tumor cells of the same histologic type as the tumor of origin. In the initial step the latter cells are transduced with a self-altering viral vector such as vesicular stomatitis virus. The genomic DNA is extracted from these cells and incorporated into the genomic DNA of vesicular stomatitis virus. This construct consisting of altered self epitopes and tumor associated antigens (Self-TAAs) altered by the viral vector is used to transduce T47EPS cells. Such T47EPS cells are delivered intravenously in vivo where their DNA comprising a broad library of tumor associated antigens (TAAs) is phagocytosed by macrophages. The altered TAAs are processed into the MHCI pathway and presented to T cells. The resultant CD8+ T cell response is directed to the altered TAAs induces a robust tumoricidal response.

In a related embodiment, the same T47EPS cells are used in vitro to transport their TAAs to macrophages. These macrophages process the TAAs into the MHC1 pathway for presentation and activation of cytotoxic CD8+ T cells. Such activated T cells are enriched and expanded in vitro as described in methods below and used for adoptive transfer into tumor bearing hosts. This approach activates a broad repertoire of individually weak T cell responses against multiple TAAs, imposing a cumulatively strong tumor specific response.

In this method, CD47 deficient erythroid progenitors to present a broad TAA repertoire from a cDNA library of a normal tissue altered by incorporation in a viral vector which can activate antitumor T cell responses. Such viral-altered self epitopes of TAAs are more immunogenic than corresponding self epitopes activating T cells that are cross reactive against both normal self, and altered self epitopes. Viruses such as vesicular stomatitis virus (VSV) are useful vectors for induction of altered self epitopes in step 1 and incorporation of DNA encoding TAAs from normal cells or tumor cells or treatment resistant tumor cells or metastatic tumor cells into their genomic DNA in step 2. Consequently, this process mobilizes a broad library of TAAs expressed from genomic DNA for efficient activation of a T cell dependent anti-tumor response in the host.

The present invention contemplates a three step process to induce a tumoricidal response. In step 1, normal cells, tumor cells and treatment resistant or metastatic tumor cells of the same histologic type as said primary tumor of origin. (collectively NTTM cells) are transduced with genomic DNA from a virus capable of altering self antigens (VASTA). While genomic vesicular stomatitis virus is preferred for this process, other vectors that alter self epitopes and whose genomic accommodates genomic DNA from NTTM cells can also be used for this purpose.

In step 2, genomic DNA is extracted from the NTTM cell and incorporated into genomic DNA of the VASTA. The latter construct is used to transduce T47EPS cells. CD47 deficient erythroid precursors, progenitors, HSCs or iPSCs are obtained from CD47 deficient mice or from erythroid precursor, progenitor, HSCs or iPSCs rendered CD47 deficient by transduction with siRNA or shRNA. When such transduced erythroid precursors reach the orthochromatic normoblast stage, the cells are collected and used for adoptive transfer into tumor bearing hosts. Such transduced cells are programmed to synthesize a broad library of TAAs.

In step 3, these transduced T47EPS are administered to tumor bearing subjects where they are phagocytosed aggressively by host macrophages which are redirected from an apoptotic to an antigen presenting and antitumor phenotype. In the host, the macrophages activated by the T47EPS stimulate a robust cytotoxic T cell response against the tumor. Such macrophages are also capable of migrating to tumor sites where they secrete IFN-γ, TNFα, IL-6, mCSF, RANTES, IL12 and nitrous oxide which contribute to the tumoricidal response.

In an additional embodiment, T cells primed in vivo by exposure to T47EPS activated macrophages are harvested from peripheral blood. They are enriched and expanded in vitro as described in methods below and returned to the host as described in methods and induce a tumoricidal response.

In still another embodiment, the invention contemplates that macrophages ingesting T47EPS present a broad TAA library to T cells in vitro. Such sensitized T cells are enriched and expanded and then adoptively transferred to tumor bearing mice where they induce a tumoricidal response.

The prior art is devoid of tangible conceptual disclosure that redirects CD47 deficient erythroid precursor cells loaded with broad library of TAA-DNA to transport their cargo to macrophages via enucleation and program such macrophages for presentation of their TAAs to T cells resulting in potent CD8+-mediated tumoricidal response.

### Methods

CD47 deficient mice are obtained from Jackson Laboratories and CD47 precursors, progenitors, HSCs or iPSCs are isolated from bone marrow of these mice as described herein. C57B1/6 mice are also used as a source of bone marrow derived erythroid precursor/progenitors or HSCs or iPSCs for knockdown of constitutive CD47 using anti-CD47 si or shRNAs as described below. Human CD47 deficient erythroid precursors, progenitors or HSCs or iPSCs are obtained from bone marrow of patients with hemophagocytois lymphohistiocytosis. CD34+ human HSCs are also obtained from human cord blood or erythroid precursors, progenitors, HSCs or iPSCs and their CD47 gene is knocked down with anti-CD47 si or shRNA. Such cells constitutively express hemoglobin AA but can also exhibit SS. SA. SC or other sickle variant hemoglobins including sickle alpha and beta thalassemia., sickle hemoglobin-C disease, sickle beta plus thalassemia, sickle hemoglobin-D disease, sickle hemoglobin-E disease, homozygous C or C-thalassemia, hemoglobin-C beta plus thalassemia, homozygous E or E-thalassemia. Indeed, any erythroblast with or without sickle hemoglobin expressing receptors capable of binding to tumor neovasculature is useful in the inventions described herein.

### Isolation and Expansion of Human CD47 Deficient Erythroid Precursors Progenitors, HSCs or IPSCs

Described below is a model method of Giarratana et al., Nat Biotech 23:69-74 (2005); however other methodology is also useful for this purpose such as that of Noulin et al., PLoS ONE 9 (11): e112496. doi:10.1371; Bird et al., PLoS ONE 9 (8): e105525. doi:10.1371). Bone marrow and peripheral blood mobilized with G-CSF via leukapheresis (LK))) is obtained from normal individuals and umbilical cord blood from normal full-term deliveries is obtained with informed consent. CD34+ cells are isolated by supermagnetic microbead selection using Mini-MACS columns (Miltenyi Biotech). Cells are cultured in a modified serum-free medium supplemented with 1% deionized BSA, 120 mg/ml iron-saturated human transferrin, 900 ng/ml ferrous sulfate, 90 ng/ml ferric nitrate and 10 mg/ml insulin (Sigma).

The expansion procedure comprises three steps. In the first step (days 0-8) 10⁴/ml CD34+ cells are cultured in the presence of 10⁶ M hydrocortisone (Sigma), 100 ng/ml stem cell factor (SCF, kindly provided by Amgen), 5 ng/ml IL-3 (R&D Systems) and 3 IU/ml erythropoietin (Eprex, kindly provided by Janssen-Cilag). On day 4, one volume of cell culture is diluted in four volumes of fresh medium containing hydrocortisone, SCF, IL-3 and erythropoietin. In the second step, the cells are resuspended at 5×10⁴, 10⁵, 2×10⁵ or 3×10⁵/ml (for cord blood, LK, bone marrow and PB cells respectively) and cocultured on an adherent stromal layer in fresh medium supplemented with erythropoietin. In the third step (up to 10 d), the cells are cultured on an adherent stromal layer in fresh medium without cytokines. The cultures are maintained at 37° C. in 5% CO₂ in air. The adherent cell layer consists of either the MS-5 stromal cell line or mesenchymal stromal cells (MSCs) established from whole normal adult bone marrow in RPMI (Invitrogen) supplemented with 10% fetal calf serum. Adherent MSCs are expanded and purified through at least two successive passages.

In a typical experiment in NOD/SCID mice 1×10⁶ CD34+ of initial leukapheresis cells are expanded to 3×10¹⁰ enucleated cells that are subsequently infused. The expansion steps are performed in bulk cultures using 10 liters of culture medium. Cells are stained with May-Grunewald-Giemsa reagent for morphological analyses, whereas enucleated cells are monitored for standard hematological variables including the MCV (fl), MCHC (%) and MCH (pg/cell) using an XE2100 automat (Sysmex, Roche Diagnostics). Semisolid culture assays. BFU-E, CFU-E and CFU-GM progenitors are assayed.
Isolation and expansion of murine CD47 deficient erythroid precursor/progenitor or HSCs (Bird et al., PLoS ONE 9 (8): e105525. doi:10.1371)

Cohorts of five, 4-6 week old female CD47 deficient mice are obtained from Jackson Laboratories (Bar Harbor, Me.). Bone marrow cells are collected from the tibia and femur bones. The bone marrow cells are washed and pelleted in a 50 ml conical tube by spinning at 1,200 RPM for 5 min. The RBCs are lysed by incubation in 5 ml sterile TAC buffer (135 mM NH₄Cl, 17 mM Tris pH 7.65). The remaining cells are washed twice in D10 media. The remaining cell pellet is resuspended in BM medium (DMEM containing 10% FCS, 100 units per ml Penn/Strep, MEM NEAA (Gibco), 10 mM HEPES, recombinant murine IL-3, IL-6, and SCF) supplemented with 5 mg/ml recombinant Tat-MYC, and 5 mg/ml recombinant Tat-Bc1-2. Cells are cultured for 28 days and then stained for cell surface markers and assessed by flow cytometry according to the antibody suppliers' protocols. Antibodies used for immunophenotypic characterization are specific for cell surface markers (Sca-1, c-Kit, Flk-2, CD150 (SLAM), CD48 (SLAM), Mac-1, GR-1, B220, TCR13, Ter119). Cohorts of 10 C57BL/6J mice are treated with 5 mg/mouse of 5-fluorouracil (5FU), administered intraperitoneally. Five days after 5FU treatment, bone marrow cells are collected from the femurs and tibias of the mice. The cells are seeded in wells of a 24 well tissue culture dish at a density of 1×10⁶ cells per well in 1 ml of medium. The cells are split into additional wells as cell density increased, to maintain a cell density at 1×10⁶ cells per well.

Preparing Human CD47 Deficient Erythroid Precursor/Progenitor/HSCs from Human Cord Blood

Fresh cord blood cells are obtained. The total volume is split into 20 ml aliquots, using 50 ml conical tubes. The 20 ml aliquots are diluted 1:1 in phosphate buffered saline (PBS). The 20 ml of diluted cord blood cells are gently overlaid over 20 ml of Ficoll-Paque Plus (Amersham Biosciences Cat #17-1440-03). The cells are spun at 9006 gravity for 60 min with the brake off. The buffy coat is removed with a glass pipette and washed twice with PBS. The cells are resuspended in CB media consisting of Iscove's media (Gibco) supplemented with 10% human plasma, 100 units per ml Penn/Strep, 60 ml of media containing SCF, IL-3 and IL-6 and 60 ml of media containing TPO, FLT3-L, and GM-CSF described above. CB media is further supplemented with 5 mg/ml recombinant Tat-MYC, and 5 mg/ml recombinant Tat-Bc1-2. Cells are incubated in 10 ml CB media with Tat-fusion proteins for 60 min at 37° C. Cells (5×10⁶ per ml) are seeded in the G-Rex 10 cell expansion device (Wilson Wolf Manufacturing) according to the manufacturer's recommendation. Cells are stained for cell surface markers and assessed by flow cytometry according to the antibody suppliers' protocols. Antibodies against cell surface markers used for stem cell characterization are (CD45, CD34, CD38, CD45RA, CD90, c-Kit, Thy 1, CD133, CD150 (SLAM), CD48 (SLAM), CD11b, B220, CD3, CD13, CD33, CD71, and GPA). In the cases where CD34+ cells are isolated, the CB cells are expanded and the CD34+ cells are then isolated using a MACS CD34+ cell isolation kit according to the manufacturer's protocol (Miltenyi Biotec).

Knockdown of CD47 Gene in Erythroid Precursors/Progenitors, HSCs or iPSCs with siRNA

CD47 expression of CD34⁺ cord blood cells is down-regulated using siRNA. CD47 siRNA primers used are as follows: siRNA1, (SEQID NO:1) UAUACACGCCGCAAUACAGAGACUC and (SEQID NO:2) GAGUCUCUGUAUUGCGGCGUGUAUA; siRNA2, (SEQID NO:3) UAGAAGUCACAAUUAAACCAAGGCC and (SEQID NO:4) GGCCUUGGUUUAAUUGUGACUUCUA. The Nucleofector kit V and Human CD34 Cell Nucleofector kit (both Amaxa) are used to transfect siRNA and transfected NB4 cells, and lineage-depleted cord blood cells are incubated for 48 and 24 hours, respectively. NB4 cells and CD34+CD38+ cells are then sorted depending on CD47 expression using the FACSAria 2 cell sorter (BD Biosciences), and these cells are used for in vitro phagocytic assays to determine the phagocytic index.
Isolation and expansion of human T cells from peripheral blood. (Rasmussen et al., J Immunol Meth 35:552-60 (2010))

Leukapheresis is performed on patients after informed consent. Following leukapheresis, lymphocytes are enriched by elutriation (ELTRA Cell Separation System, Gambro BCT). PBMCs are separated by density gradient centrifugation (Lymphoprep™, Axis-Shield, Norway) and monocytes are depleted using Dynabeads CD14 as described by the manufacturer. PBMCs used for expansion of virus specific T cells are collected from HLA-A2 positive healthy donors and patients with various malignant tumors as described herein. Expansion of tumor specific T cells is performed on PBMC from a stage IV malignant melanoma patient, isolated from blood samples taken 4 weeks following vaccination with CD47 deficient erythroblasts. After vaccination and prior to expansion, the patient displayed 0.54% hTERT+CD8+ T cells, while no detectable levels of hTERT+CD8+ T cells is detected prior to vaccination. Prior to expansion of virus specific T cells, CD25+ cells are depleted using Dynabeads CD25 following the manufacturer's instructions. Briefly, the CD4+ T cell numbers are determined by flow cytometry at the time of leukapheresis and Dynabeads CD25 are added at a CD4⁺ T cell-to-beads ratio of 2:1 in thawed samples (assuming 2.5-5% CD4+CD25hi T cells, which gives 10-20 beads per target cell). Incubation is performed at 4° C. for 30 min and CD25⁺ cells are captured with a magnet (MPC-6, Invitrogen Dynal, Norway).

Monocyte-depleted PBMC are stimulated with two different types of beads; Dynabeads ClinExVivo CD3/CD28 and Dynabeads CD3low/CD28. T cells (1×10⁶ CD3+ T cells/well) are cultured in 24-well tissue culture plates at 37° C., 5% CO2 at a concentration of 0.7×10⁶/mL. When cell numbers reach 1.7×10⁶/mL the cells are transferred to culture flasks (Nunclon™ Surface, Nunc™, Denmark). From day 3 the cells are counted daily and the viability is evaluated by Trypan Blue (Gibco, Invitrogen, CA) staining. Fresh media is added daily to maintain a cell concentration of 0.5×10⁶ cells/ml. Flow cytometric analysis is performed before activation, on days 7 and 10 of the expansion. The number of Ag-specific CD8+ T cells is calculated as percentage of the total number of CD8+ cells. In separate experiments, PBMC are stimulated with CMVpp65 (SEQID NO:5) (NLVPMVATV) or hTERT (SEQID NO:6) (ILAKFLHWL) peptides at a final concentration of 5 μg/mL (Proimmune Ltd, UK) and cells are cultured for 10-14 days in media supplemented with 20 U/mL IL-2 and 5 ng/mL IL-7.

T cells are cultured in CellGro® Serum-free Culture Media (CellGenix, Freiburg, Germany) supplemented with 5% heat inactivated human serum (PAA Laboratories GmbH, Austria), 10 mM N-acetylcysteine (Mucomyst 200 mg/mL, AstraZeneca AS, Norway), gentamycin 0.05 mg/mL (Garamycin 40 mg/mL, Schering-Plough Europe, Belgium) and 100 U/mL recombinant human interleukin-2 (IL-2) (Proleukin®, Novartis). Recombinant human interleukin-7 (IL-7) is purchased from BD Pharmingen™. Anti-CD3/anti-CD28 coupled Dynabeads (Dynabeads® ClinExVivo™ CD3/CD28, and Dynabeads® CD3low/CD28), Dynabeads® CD4, Dynabeads® CD14, and Dynabeads® ClinExVivo™ CD25 are kindly provided by Dynal Invitrogen, Oslo, Norway. Flu vaccine was a mix of Brisbane/59/2007 strain H1N1 and Brisbane/10/2007 strain H3N2 (Solvay Biologicals BV, Netherland). The following fluorochrome-conjugated mAbs are used for flow cytometric analyses: FITC-conjugates of anti-CD4, CD8, CD27, CD28, CD57 (Dako Cytomation, Denmark), anti-CD3, CD14 (BD Biosciences, San Jose, Calif.), anti-CCR7 and CD62L (R&D Systems, Minneapolis, Minn.); phycoerythrin (PE) conjugates of anti-CD4, CD25 (Dako Cytomation, Denmark); PE-Cy-5 conjugate of anti-CD8 (Dako Cytomation, Denmark); APC-conjugate of CD25, PerCP-conjugate of CD4; R-PE labeled HLA A*0201 pentamers specific for CD8+ CMVpp65 (SEQID NO:5) (NLVPMVATV), EBV BMLF1 (SEQID NO:7) (GLCTLVAML), and hTERT (SEQID NO:6) (ILAKFLHWL) are purchased from ProImmune Ltd (UK). Cells were stained with fluorochrome-labeled mAbs for 20 min at room temperature in 50 μL staining buffer (PBS+0.1% human serum albumin (Octapharma, Stockholm, Sweden)+0.1% NaN3) and 54, of 10 mg/mL gamma globulin (Gammagard, Baxter, UK). Pentamer staining is performed as described by the manufacturer. Briefly, cells are stained with pentamers for 15 min at room temperature prior to staining with other mAbs as described above. Samples are analyzed on a FACScan or LSR II cytometer (Becton Dickinson, Franklin Lakes, N.J.) using FlowJo software (Tree Star Inc.).

Genomic DNA from Normal Tissue, Primary Tumor and Metastatic Tumor Cells;

In step 1 tumor cells, normal tissue cells or tumor cells from primary or metastatic or treatment resistant tumors are transduced with a virus that induces changes in self protein. The archetypical example of such a virus is vesicular stomatitis virus. After a sufficient period to allow viral integration into the host cell genome, the genomic DNA from each individual population of cells is extracted from these cells and incorporated individually into separate genomic DNA constructs of the vesicular stomatitis virus. The construct to be used in any individual patient is based on whether the patient displays a primary, metastatic or treatment resistant tumor. Normal tissue DNA can be used to treat patients with primary tumors. While genomic VSV is preferred, a lentiviral or other suitable vector that can accommodate this genomic DNA can also be used for this purpose. GVNT-DNA is extracted from normal tissues such as prostate, lung, breast, kidneys and colon and from primary and metastatic carcinomas of the prostate, lung, breast, kidneys and colon are extracted as disclosed in U.S. patent application Ser. No. 13/328,748 filed Dec. 16, 2011 incorporated in entirety by reference with its references. The DNA extracted from these cells is used with a 5000 base pairs cut-off and integrated into the open reading frame of the vesicular stomatitis virus genomic DNA by the method of Fernandez M et al, J Virol 76: 895-904 (2002) incorporated by reference with its references in entirety.

Tumor cells transduced by the virus are derived from an established tumor cell line having an identical tissue type as the tumor of said tumor-bearing subject. It need not be HLA class II matched to said subject. Further, the tumor cells are obtained, for example, from a solid tumor of an organ, such as a tumor of the lung, liver, breast, colon, bone, etc. The tumor cells are also secured from a blood-borne i.e., dispersed or metastatic malignancy, Tumor cells are acquired from a subject by, for example, surgical removal of tumor cells, e.g., a biopsy of the tumor, or from a blood sample from the subject in cases of blood-borne malignancies. In the case of an experimentally induced tumor, the tumor cells used to induce the tumor can be used, e.g., cells of a tumor cell line. Tumor samples of solid tumors are treated prior to modification to produce a single-cell suspension of tumor cells for maximal efficiency of transfection. Possible treatments include manual dispersion of cells or enzymatic digestion of connective tissue fibers, e.g., by collagenase. The tumor cells are transfected immediately after acquisition from the subject or cultured in vitro prior to transfection to allow for expansion and further characterization of the tumor cells (e.g., determination of the expression of cell surface molecules).

The tumor cells of the present invention which in step 1 are transfected with the virus comprise any primary or metastatic tumor cell including but not limited to those derived from carcinomas, sarcomas, lymphoma, glioma, melanoma, neuroblastoma and the like. Examples of effective transfection methods include electroporation, calcium-phosphate precipitation, DEAE-dextran treatment, lipofection, microinjection and infection with viral vectors. These methods of transfection of mammalian cells are well-known in the art, and are described, e.g., in Sambrook et al, Molecular Cloning: A Laboratory Manual, 2nd Edition, Cold Spring Harbor Laboratory press (1989)).

Tumors that recur after chemotherapy, radiation and immunotherapy and show a different phenotype from the original tumor are also useful in this invention. Indeed, chemotherapy, radiation and immunotherapy resistant tumors lose, or show reduced expression of state-specific antigens in the original tumor as well as increased expression of N-cadherin, SNAIL and SLUG, associated with an epithelial-mesenchymal-like transition.

Normal or tumor cells of the same histologic type or metastatic or drug resistant tumor of the same histologic type as the tumor of origin are also useful in this invention. They can be syngeneic, allogeneic or xenogeneic to the host. As with primary and metastatic tumor cells such normal cells are transduced with the viral construct, their cDNA extracted and integrated into a virus or viral genomic DNA for transfection into erythroid progenitor cells.

In step 1, the present invention contemplates infecting these primary or recurrent or metastatic tumors cells and or normal tissue cells with a viral vector and extracting the cDNA from these cells for use in step 2. The viruses that are useful for transducing these cells are described below.

Viruses that Alter Self/TAA Antigens (VASTA) in Tumor Cells and Normal Cells of the Same Histologic Type or Metastatic or Drug Resistant Tumor of the Same Histologic Type as the Tumor of Origin

Viruses (vectors or genomic DNA) are used for infection of tumor cells or normal cells of the same histologic type or metastatic or drug resistant tumor of the same histologic type as the tumor of origin. These viruses are selected for their ability to alter self/tumor antigens and render them immunogenic. Molecules encoded within the viral vector are expressed efficiently in cells which have taken up viral vector nucleic acid. The viral nucleic acid may be a DNA or RNA molecule as long as it retains the ability to alter and express self/tumor antigens. In step 1 above the live virus or its genomic DNA encoding are transfected into tumor cells or normal cells. In step 2 the DNA extracted from such tumor cells or normal cells is extracted and inserted into the genomic DNA of the same or a different virus. Viruses or their genomic DNA described below are useful in both steps 1 and 2.

Viruses useful for this purpose include any virus capable of altering self-antigens in tumor cells and normal cells such that they elicit an immune response in the host. To date, this property has been demonstrated for vesiclular stomatitis virus. Nevertheless, in view of their ability to integrate into the genome of tumor cells they are potential candidates to alter self/tumor antigens and render them immunogenic to the host. A preferred vector for steps 1 and 2 of the present invention is a recombinant virus which is capable of efficient delivery of genes to multiple cell types, including normal cells and tumor cells, altering self/tumor antigens in such transduced cells and increasing their immunogenicity. Such viruses with these properties are collectively referred to as VASTA.

Vesicular stomatitis virus (VSV) or its genomic DNA is the archetypical VASTA. It displays an adjuvant effect when combined recombinantly with other antigens including TAAs. VSV is used to transduce primary or metastatic or treatment resistant tumor cells or normal cells in step 1. It is also useful in step 2 wherein the site between genes G and L of the vesicular stomatitis virus (VSV) genomic plasmid pVSV-XN2 is utilized for insertion of genomic-DNA extracted from tumor cells or normal cells transduced with VSV in step 2. The recombinant method for insertion of a foreign gene in genomic VSV is described in U.S. patent application Ser. No. 13/328,748 filed Dec. 16, 2011 and Fernandez M et al, J Virol 76: 895-904 (2002) incorporated by reference.

Other viruses with the above properties are also useful in Steps 1 and 2. Herpes Simplex virus type 1 (HSV-1) deleted for ICP34.5, provides tumor-selective replication, and ICP47 deletion increases US 11 expression, which enhances virus growth and replication in tumor cells normal cells. In the present invention nucleic acids encoding this HSV mutant with deleted ICP34.5 region is used in step 1 used to transduce tumor cells or normal cells. cDNA extracted from such cells is incorporated into genomic DNA of VSV (VSV virus) or genomic DNA of HSV (or HSV virus) and used for administration to the host as described herein.

Similarly, step 1 may comprise JX-594, a replication-competent Wyeth strain vaccinia virus. In step 2, this virus is genetically modified to integrate Gntv-DNA under the control of a synthetic early/late promoter. The genes are initially cloned into the Sail and Bglll sites of the plasmid transfer vector pSC65. Poxviruses are also useful in the present invention include replicating and non-replicating vectors such as orthopox, vaccinia, raccoon pox, rabbit pox and the like, avipox, suipox, capri-pox and the like. Poxviruses may be selected from the group consisting of vaccinia-Copenhagen, vaccinia-Wyeth strain, vaccinia-MVA strain, NY VAC, fowlpox, TROVAC, canarypox, ALVAC, swinepox, and the like.

Additional viral vectors useful in this invention include but are not limited to adenovirus, alphavirus, retrovirus, picornavirus, iridovirus, self-replicating RNA replicons (replicase nucleic acids) derived from alphavirus vectors, such as Sindbis virus, Semliki Forest virus, or Venezuelan equine encephalitis viruses. Insertion method of SAg into adenovirus variant is disclosed in USPA 810/428,817-Example 60 incorporated by reference. Lentiviral vectors capable of incorporating nucleic acids of three or more molecules (Zuffrey et al, Nature Biotech 15: 871-875 (1997)) incorporated herein by reference are useful in the present invention. Other viruses that have natural core engineered properties (Kim et al. Nat. Med. 7: 781-187 (2001); Alemany et al, Nat. Biotechnology 18: 723-730 (2000)) incorporated by reference in entirety are useful in this invention.

The recombinant vector of the present invention comprises at least one expression control element operably linked to the nucleic acid sequence. The expression control elements are inserted into the vector to control and regulate the expression of the nucleic acid sequence (Ausubel et al, 1987, in “Current Protocols in Molecular Biology, John Wiley and Sons, New York, N.Y). Expression control elements are known in the art and include promoters. Promoters useful in the present invention are the SV40 (simian vims 40) early promoter, the RSV (Rous sarcoma vims) promoter, the adenovirus major late promoter, the human CMV (cytomegalovims) immediate early I promoter and poxvirus promoters

The recombinant vector of the present invention is able to infect, transfect or transduce host cells in a mammal. The host cells are any cell amenable to infection, transfection or transduction by the recombinant vector or VASTA and capable of expressing the foreign genes from the recombinant vector at functional levels. The host cells include but are not limited to any normal or tumor cells of the same histologic type or metastatic or treatment resistant tumor cell of the same histologic type as the tumor of origin. Such cells can be autologous, allogeneic or xenogeneic to the host. Such cells are obtained from cell cultures, whole blood or from biopsies of tumor or normal tissues including lymph nodes. Normal cells include fibroblasts, muscle cells, APCs and antigen presenting precursor cells such as monocytes, macrophages, DC, Langerhans cells and the like.

Infection of the host cells allows expression of each foreign, exogenous molecule and expression of the foreign nucleic acid sequence encoding target tumor antigen(s) if present in the recombinant vector. The host cells express, or are engineered to express, the appropriate MHC (HLA) Class I or II molecules for appropriate antigenic presentation to CD4⁺ and/or CD8⁺ T cells. As such virtually any mammalian cell may be engineered to become an appropriate antigen presenting cell expressing multiple tumor antigenic and altered self molecules.

cDNAs containing a CpG backbone from normal or tumor cells of the same histologic type or metastatic or treatment resistant tumor cell of the same histologic type as the tumor of origin. Tumor cells transduced with VASTA are extracted as described in Example 2 of U.S. patent application Ser. No. 13/328,748. The extracted cDNA or RNA is then integrated into a VASTA as described in Example 2 of U.S. patent application Ser. No. 13/328 used as preventative or therapeutic vaccine as in animal models and humans as in Examples 3, 4, 5, 6, 7 of USPA '748 which is incorporated by reference with its references in entirety.

Table 1 from U.S. patent application Ser. No. 13/328,748 filed Dec. 16, 2011 incorporated by reference in entirety provides several viruses for use in step 1 and their insertion sites for nucleic acids for use in step 2.

Transduction of C47 Deficient Erythroid Precursors, Progenitors, HSCs iPSCs or Macrophages with DNA from Viral Infected Normal and Tumor Cells.

In step 1, Normal cells, primary tumor cells of the same histologic type and treatment resistant or metastatic tumor cells of the same histologic type as said normal cells and said primary tumor of origin (collectively NTTM cells):are transduced with vesicular stomatitis virus (VSV) or genomic VSV DNA. Such NTTM cells may be autologous, allogeneic or xenogeneic to the host.

In step 2, cDNA libraries from virus transduced NTTM cells are extracted with phenol and ethanol-precipitated as described in U.S. patent application Ser. No. 10/428,817-Example 30. These cDNA libraries are cloned into the pCMV. SPORT6 vector (Invitrogen), and amplified by PCR. The cDNA library from each cell type is then cloned into pVSV-XN2 between the Xho 1 and Nhe 1 sites between the G and L genes of genomic VSV. The latter consists of 4.75×10⁶ colony-forming units (at dilutions of 1×10⁻⁶ and 1×10⁻⁵ there are five and 45 colonies, respectively). Virus is generated from BHK cells by cotransfection of pVS V-XN2-cDNA library DNA along with plasmids encoding viral genes. Virus is expanded by a single round of infection of BHK cells and purified by sucrose gradient centrifugation. cDNA libraries generated from the tumors or normal cells for insertion into the VSV genomic DNA are size-fractionated to PCR cDNA molecules below 4 kilo-base pairs (kbp), as smaller cDNA inserts are associated with both higher viral titers and lower proportions of defective interfering particles.

Genomic DNA from normal human cells is amplified from the BioExpress shuttle vector by PCR and cloned into the VSV genomic plasmid pVSV-XN2 between the G and L genes. Virus is generated from BHK cells by cotransfection of pVS V-XN2-cDNA library DNA along with plasmids encoding viral genes as described. Titers are measured by plaque assays on BHK-21 cells. All constructs are tested in vitro to validate their ability to express the desired gene product. Plasmids purified by column (Wizard preps, Promega, Madison. Wis.) or by cesium 5 chloride banding are used to transfect tissue-culture cells transiently. Protein expression is detected by immunoblot. This check not only verifies expression but validates the size and immunoreactivity of the gene product.

The VSV genomic DNA containing the genomic DNAs from viral transduced NTTM cells is are used individually to transduce individual populations of CD47 deficient erythroid precursors, progenitors, HSCs or iPSCs (collectively T47EPS cells after this transduction).

The T47EPS cells are tested in animal models cancer as described in the section on Tumor Models. Typically inbred mice such as C57BL/6mice are purchased from Jackson Laboratories at 6-8 weeks of age. Subcutaneous tumors are established by injection of 2×10⁶ tumor cells in 100 μL PBS into the flank. Transduced erythroid cells (10⁶-10⁷) are delivered intravenously 1-10 days before tumor implantation. Tumor growth and responses are assessed as described in Tumor Models section.

In another embodiment, macrophages obtained from bone marrow are incubated with transduced erythroid progenitors in vitro for 3-7 days. When the macrophages have engulfed nuclei from erythroid progenitors they are isolated and incubated with CSF for an additional 3-12 days after which they are cocultured with syngeneic T cells in vitro for an additional 2-5 days. The syngeneic T cells are expanded, collected then administered intravenously in doses of 10⁶-10⁷ cells. T cell for use in this invention are also derived from iPPS cells or embryonic stem cells using methods well defined in the art.

For treatment of humans, CD47 deficient erythroid precursors, progenitors, HSCs or iPSCs are administered individually via a parenteral route preferably intravenously and preferably on alternate days for 15 up to 30 days per cycle. Erythroid precursor, progenitor, HSCs or iPSCs transduced with DNA extracted from primary tumors may be used together with erythroid precursors, progenitors or HSCs or iPSCs are transduced with DNA from normal tissues from which the tumor originated. Erythroid precursors, progenitors, HSCs or iPSCs transduced with DNA from metastatic or treatment resistant tumors are used to treat tumors that are recurrent despite treatment (as defined herein) or metastatic tumors expressing molecules such as cadherin, adhesion and metalloproteinases not present in the primary tumor or normal tissue of origin.

### In Vivo Treatment.

Erythroid precursors, progenitors HSCs or iPSCs (10⁶) transduced with genomic DNAs from normal cells or the various tumor cells described in the previous sections are injected intravenously into C57BL/6 mice bearing Lewis lung tumor or B 16 melanoma implanted subcutaneously (10⁶ tumor cells) 6 days earlier having reached a diameter of at least 5 mm. Erythroid cell infusions are repeated every other day for a total of 3-5 injections. Tumors are measured daily and the mice are sacrificed when tumors reach a volume of 1500 mm³ as described in the section on Tumor Models and Methods.

Similarly, in a second group of tumor bearing mice, macrophages that phagocytosed the genomic DNAs from normal cells or the various tumor cells described in the previous sections are harvested and 10⁵ cells are injected intravenously. In still a third method, expanded macrophages that phagocytosed DNA from the erythroid precursors, progenitors, HSCs or iPSCs transduced with genomic DNAs from normal cells or the various tumor cells are incubated with T cells obtained from the same or allogeneic donors and co-cultured for 7-10 days. The T cells are expanded in vitro with IL-2 and IL-15, harvested and used for adoptive transfer intravenously in doses of 10⁵-10⁶ cells. The erythroid precursors, progenitors, HSCs or iPSCs from step 1 or macrophages from step 2 or T cells from step 3 are injected or infused into tumor bearing mice every other day for a total of 1-12 treatments. Control mice are similarly implanted with tumors but receive saline treatment instead of erythroid cells, macrophages or T cells. Use of these cells in human clinical trials is given in Examples 2, 7 and 8.
T cell infusions in humans (Ellebaek et al., J Transl Med 10:169-176 (2012)

Patients between ages of 18-70 years with metastatic malignant melanoma are included. Further inclusion parameters are disease in progression (according to response evaluation criteria in solid tumors (RECIST)), measurable disease according to RECIST, at least one resectable metastasis at a minimum of 1 cm³, and performance status (PS)≦1. Exclusion criteria are significant heart or lung disease, autoimmune disease, other neoplastic tumors within the last 5 years, treatment with steroids, or involvement of the central nervous system. All patients sign a written informed consent before entering the study.

Patients are admitted to hospital at day −8 and a central venous catheter is applied. A lymphodepleting chemotherapy regimen consisting of Cyclophosphamide, 60 mg/kg/d day −7 to −6 and fludarabine phosphate, 25 mg/m²/d at day −5 to −1 administered as previously described [10]. Prophylactic antiemetic Domperidone is given together with pantoprazole. On day 0, autologous T cells 0.3-7.5×10¹⁰ are infused intravenously followed by 14 days of subcutaneous IL-2 injections, 2 MIU, starting the same evening. Patients are treated prophylactically with trimethoprim, sulfamethoxazole, and acyclovir from the beginning of treatment and 6 months thereafter and with fluconazole during the leukopenic period. Clinical response is monitored with a computed tomography (CT) or positron emission tomography (PET)/CT scan 8 weeks after T cell infusion and assessed according to RECIST 1.0.
Isolation and expansion of human macrophages from peripheral blood (Kuriyama et al., Blood 120 4058-4067 (2012); Hennemann et al. Cancer Immunol. Immunother 45: 250-256 (1998)

Monocytes are purified from PBMCs by positive selection using MACS CD14 Micro Beads (Miltenyi Biotec). These cells are incubated with X-VIVO10 (Lonza) containing 2% AB serum and 100 ng/mL of M-CSF (R&D Systems) for more than 96 hours to obtain macrophages. Monocytes are also collected by leukapheresis with a continuous-flow cell separator (CS 3000; Baxter, Munich, Germany) peripheral blood mononuclear cells (PBMC) separated from red blood cells and granulocytes by Ficoll-Hypaque gradient centrifugation (IBM 2991, Cobe Laboratories, Heimstetten, Germany) and then loaded onto an elutriation system (Beckman J6-ME, JE-5.0 rotor with a standard elutriation chamber of 40 ml) to remove lymphocytes. Monocytes remaining in the elutriation chamber are collected and resuspended at 5×10⁶ cells/ml in RPMI-1640 medium, supplemented with amino acids, polyvitamins, pyruvate, mercaptoethanol, penicillin, streptomycin, and 2% autologous serum. Cells are seeded into hydrophobic Teflon bags (Biofolie 25, Heraeus GmbH, Germany) and cultured at 37° C. and 5% CO₂. IFN-γ (Polyferon, Bioferon GmbH, Laubheim, Germany) is added 18 h prior to harvest to give a final concentration of 200 U/ml. After 5±9 days of culture, the cells are harvested, washed twice with phosphate-buffered saline, resuspended in 100 ml clinical-grade 5% human albumin and infused i.v. within 30 min.

Pulsing of Macrophages with DNA from T47EPS Cells

T47EPS cells are incubated with 10⁴ bone marrow derived macrophages for 4 h. After incubation, 1×10⁶ macrophages are washed twice in complete media resuspended in fresh RPMI-1640 medium containing 5% FCS. The supernatants are collected for the assay of human M-CSF and murine IFNγ contents by ELISA (Endogen, Cambridge, Mass., USA).
Treatment of tumor-bearing mice with macrophages pulsed with DNA from T47EPS cells (Lei et al., Gene Ther 7, 707-713 (2000))
   Macrophages transduced with DNA from T47EPS cells are used. C57BL/6 mice given 1×10⁵ B16F10 melanoma cells subcutaneously on day 1. When the tumors reach a diameter of 0.75 mm (day 7 after tumor implant) each group is injected intravenously with 10⁶ transduced macrophages as described above in 50 uL PBS. The tumors are measured daily and results recorded and analyzed as described in the section on Tumor Models.
Treatment of humans with macrophages pulsed with DNA from T47EPS cells (Andreesen et al., Cancer Res 50:7450-7456 (1990))

Human macrophages transduced with DNA from human T47EPS cells are administered to patients with measureable breast carcinoma lesions. Such transduced macrophages (10⁶-10¹¹) are administered into either large peripheral or central veins over a period of 10-20 min or i.p. using an implanted Port-a-Cath system. This is followed by the infusion of an additional 250 ml of physiological saline. If ascites was present, it is drained as completely as possible prior to cell infusion. Infusions are repeated every 2-5 days for four weeks. Tumor volume is measured weekly and responses are recorded and analyzed by the RECIST criteria.

Treatment of Macrophages with Opsonizing Antibodies and/or Complement Components Before or Concomitant with Addition of T47EPS Cells

In some instances macrophage polarization in response to a CD47 deficient erythroblast may require a second signal in addition to CD47 deficiency provided by T47EPS cells. Such a signal is provided by opsonizing antibodies directed to antigens expressed by CD47 deficient erythroid precursors/progenitors/stem cells. The Fc fragments of such opsonizing antibodies activate macrophage FcγR dependent erythrophagocytosis. Such antibodies can be of the IgM or IgG class such IgG1a (e.g., murine 105-2H) and IgG2a (e.g., murine 34-3c) Likewise, multiple complement components and fragments that attach to CD47 deficient erythroblasts are also capable of activating complement receptors and polarizing macrophages into an antigen presentation phenotype. Methodology for use of such opsonizing antibodies with CD47 deficient erythroid cells is provided by Oldenborg et al., Blood supra (2002) which is herein incorporated by reference in entirety with its references.

### Chemotherapeutic Agents Augment the Antitumor Effects of SSRBC Therapeutic Compositions, Microparticles and Liposomes

These PS expressing liposomes comprising heme or hemoglobin may also incorporate various chemotherapeutics, anti-tumor toxins and biologic agents. Any one or more of genes, vectors, antisense constructs, siRNA constructs, and ribozymes, as appropriate, may be incorporated into the liposomes.

A variety of chemotherapeutic and pharmacological agents may also be given separately. The chemotherapeutic agent(s) selected for therapy of a particular tumor preferably is one with the highest response rates against that type of tumor. For example, for non-small cell lung cancer (NSCLC), cisplatin-based drugs have been proven effective. Other agents useful in NSCLC include the taxanes (paclitaxel and docetaxel), vinca alkaloids (vinorelbine), antimetabolites (gemcitabine), and camptothecin (irinotecan). For breast cancer, a microtubule inhibitor such as taxotere is the preferred. For malignant ascites due to gastrointestinal tumors, 5-FU is preferred. The optimal chemotherapeutic agents and combined regimens for all the major human tumors are set forth in Bethesda Handbook of Clinical Oncology, 4th edition, Abraham J et al., Wolters Kluwer/Lippincott William & Wilkins, Philadelphia, Pa. (2014); Manual of Clinical Oncology, Fourth Edition, Casciato, D A et al., Lippincott William & Wilkins, Philadelphia, Pa. (2000) both of which are herein incorporated in entirety by reference.

A particularly notable agent for synergy with the various therapeutic PSSSRBCs and PS exposing liposomes is Gemcitabine. The latter is a broad-spectrum antimetabolite and deoxycytidine analogue with antineoplastic activity. Upon administration, gemcitabine is converted into the active metabolites difluorodeoxycytidine diphosphate (dFdCDP) and difluorodeoxycytidine triphosphate (dFdCTP) by deoxycytidine kinase. dFdCTP competes with deoxycytidine triphosphate (dCTP) and is incorporated into DNA. This locks DNA polymerase thereby resulting in \masked termination\ during DNA replication. These agents are used separately 1-5 days before, during or 1-7 days after administration of liposomes or the various PSSSRBC preparations.

Other agents that are operable inside or used separately before, during or after the therapeutic liposomes of various PSSSRBC cells include, radiotherapeutic agents, antitumor antibodies with attached anti-tumor drugs such as plant-, fungus, or bacteria-derived toxins or coagulants, ricin a chains, deglycosylated ricin a chains, ribosome inactivating proteins, sarcins, gelonin, aspergillin, restricticin, ribonuclease, epipodophyllotoxin, diphtheria toxin, superantigens and superantigen homologues or superantigen fusion proteins or pseudomonas exotoxin. Additional cytotoxic, cytostatic or anti-cellular agents capable of killing or suppressing the growth or division of tumor cells include anti-angiogenic agents, apoptosis-inducing agents, coagulants, prodrugs or tumor targeted forms, tyrosine kinase inhibitors (Siemeister et al., 1998), antisense strategies, RNA aptamers, miRNAs, siRNA and ribozymes or antibodies against VEGF or VEGF receptors (Saleh et al., 1996; Cheng et al., 1996; Ke et al., 1998; Parry et al., 1999; each incorporated herein by reference). Any of a number of tyrosine kinase inhibitors are useful. These include, for example, the 4-aminopyrrolo[2,3-d]pyrimidines (U.S. Pat. No. 5,639,757). Further examples of small organic molecules capable of modulating tyrosine kinase signal transduction via the VEGF-R2 receptor are the quinazoline compounds and compositions (U.S. Pat. No. 5,792,771). Additional agents which may be employed are steroids such as the angiostatic 4, 9(11)-steroids and C21-oxygenated steroids (U.S. Pat. No. 5,972,922). Thalidomide and related compounds, precursors, analogs, metabolites and hydrolysis products (U.S. Pat. Nos. 5,712,291 and 5,593,990) to inhibit angiogenesis. Interferons and metalloproteinase inhibitors are two other classes of naturally occurring angiogenic inhibitors that can be used. Vascular tumors in particular are sensitive to interferon; for example, proliferating hemangiomas are successfully treated with IFN. Tissue inhibitors of metalloproteinases (TIMPs), a family of naturally occurring inhibitors of matrix metalloproteases (MMPs), can also inhibit angiogenesis.

Drugs that inhibit HIF-1 such as doxorubicin are especially useful in this invention (Table 2). Similarly microtubular drugs that disrupt endothelium such as combretastatin are also useful. These drugs like all of the others listed above may be delivered alone or in combination 1-5 days before, during or 1-7 days after administration of SSRBCs, PSSSRBCs, 47SSRBCs, PS47SSRBCs or SSRBC-derived microparticles or liposomes described herein.

TABLE 2
Inhibitors of HIF-1

| Mechanism of action | Inhibitor |
|---|---|
| Inhibition of | Aminoflavone, GL331 |
| HIF-1α mRNA | |
| Inhibition of HIF-1α | Topoisomerase inhibitors (such as rapamycin, |
| protein synthesis | temsirolimus, and everolimus); cardiac |
| | glycosides (such as digoxin and digtoxin), |
| | microtubule targeting agents (such as 2- |
| | methoxyestradiol, epothilone B, and taxotere), |
| | topoisomerase inhibitors (such as camptothecin, |
| | topotecan, and NSC 644221), oligonucleotides, |
| | AF, PX-478 |
| Inhibition of HIF-1α | HSP90 inhibitors (such as 17-allylamino- |
| protein stabilization | demethoxygeldanamycin, 17- |
| | dimethylaminoethylamino-17- |
| | demethoxygeldanamycin, and apigenin), HDAC |
| | inhibitors (such as FK228 and LAQ824), |
| | antioxidants (such as ascorbic acid), |
| | oligonucleotides, selenium compound (such as |
| | berberine and methylselenocysteine); PX-12,YC-1 |
| Inhibition of | Acriflavine |
| HIF-1α:HIF-1β | |
| dimerization | |
| Inhibition of | Anthracyclines, echinomycin |
| HIF-1:DNA binding | |
| Inhibition of | Bortezomib |
| HIF-1 transactivity | |

Heme oxygenase inhibitors of various types including zinc protoporphyrin are used up to two weeks before, together with or up to two weeks after administration of PS-SSRBCs or PS47SSRBCs or PSMPS or PS liposomes. Doses recommended for humans, preparation of these agents, pharmaceutical vehicles and therapeutic usage are provided in U.S. Pat. No. 8,431,117 which is incorporated by reference with its references.

### Administration

Compositions comprising the cells of the invention (e.g., erythroid precursors (erythroblasts), progenitors, HSCs or iPSCs or macrophages or T cells) are administered systemically or directly into a tumor. Expansion and differentiation agents are provided prior to, during or after administration of the cells to increase production of erythroblasts or T cells or macrophages in vitro or in vivo. The modified cells are administered intravenously in any physiologically acceptable vehicle. Usually, at least 1×10⁵-1×10¹⁰ cells are administered. Genetically modified immunoresponsive cells of the invention can comprise a purified population of cells. Those skilled in the art can readily determine the percentage of cells in a population using various well-known methods, such as fluorescence activated cell sorting (FACS). Preferable ranges of purity in populations are about 50% to about 55%, about 55 to about 60%, and about 65 to about 70%. More preferably the purity is about 70 to about 75%, about 75 to about 80%, about 80 to about 85%; and still more preferably the purity is about 85 to about 90%, about 90 to about 95%, and about 95 to about 100%. Dosages can be readily adjusted by those skilled in the art (e.g., a decrease in purity may require an increase in dosage).

The cells are introduced by injection, catheter, or the like. If desired, factors can also be included, including, but not limited to, interleukins, e.g. IL-2, IL-3, IL-6, and IL-11, as well as the other interleukins, the colony stimulating factors, such as G-, M- and GM-CSF, interferons, e.g. .gamma.-interferon, stem cell factor and erythropoietin.

Compositions of the invention include pharmaceutical compositions and a pharmaceutically acceptable carrier. Administration is autologous or allogeneic. Cells can be obtained from one subject, and administered to the same subject or a different, compatible subject. Peripheral blood derived cells of the invention or their progeny are administered via, systemic injection, localized injection, intravenous injection, or parenteral administration. When administering a therapeutic composition of the present invention (e.g., a pharmaceutical composition), it is generally be formulated in a unit dosage injectable form (solution, suspension, emulsion).

### Tumor Models and Procedures for Evaluating Anti-Tumor Effects Studies

The PS and PS47SCs, CD47Es, T cells and macrophage compositions and self-stimulating T cells described herein are tested for therapeutic efficacy in several well established rodent models which are considered to be highly representative of a broad spectrum of human tumors. These approaches are described in detail in Geran, R. I. et al., “Protocols for Screening Chemical Agents and Natural Products against Animal Tumors and Other Biological Systems (Third Edition)”, Canc. Chemother. Reports, Pt 3, 3:1-112, which is hereby incorporated by reference in its entirety.

### A. Calculation of Mean Survival Time (MST)

MST (days) is calculated according to the formula:

$$\frac{S + AS ( A \text{-} 1 ) - ( B + 1 ) N T}{S ( A \text{-} 1 ) - N T}$$

Day: Day on which deaths are no longer considered due to drug toxicity. For example, with treatment starting on Day 1 for survival systems (such as L1210, P388, B16, 3LL, and W256): Day A=Day 6; Day B=Day beyond which control group survivors are considered “no-takes.”
S: If there are “no-takes” in the treated group, S is the sum from Day A through Day B. If there are no “no-takes” in the treated group, S is the sum of daily survivors from Day A onward.
S(A-1): Number of survivors at the end of Day (A−1).
Example: for 3LE21, S(A-1)=number of survivors on Day 5.
NT: Number of “no-takes” according to the criteria given in Protocols 7.300 and 11.103.

### B. T/C Computed for all Treated Groups

$$T / C = \frac{M S T of treated group}{M S T of control group} \times 100$$

Treated group animals surviving beyond Day Bare eliminated from calculations (as follows):

| No. of survivors in treated | Percent of “no-takes” | |
|---|---|---|
| group beyond Day B | in control group | Conclusion |
| 1 | Any percent | “no-take” |
| 2 | <10 | drug inhibition |
| | ³10 | “no-takes” |
| ³3 | <15 | drug inhibitions |
| | ³15 | “no-takes” |

Positive control compounds are not considered to have “no-takes” regardless of the number of “no-takes” in the control group. Thus, all survivors on Day B are used in the calculation of T/C for the positive control. Surviving animals are evaluated and recorded on the day of evaluation as “cures” or “no-takes.”

### Calculation of Median Survival Time (MedST)

MedST is the median day of death for a test or control group. If deaths are arranged in chronological order of occurrence (assigning to survivors, on the final day of observation, a “day of death” equal to that day), the median day of death is a day selected so that one half of the animals died earlier and the other half died later or survived. If the total number of animals is odd, the median day of death is the day that the middle animal in the chronological arrangement died. If the total number of animals is even, the median is the arithmetical mean of the two middle values. Median survival time is computed on the basis of the entire population and there are no deletion of early deaths or survivors, with the following exception:

C. Computation of MedST from Survivors

If the total number of animals including survivors (N) is even, the MedST (days) (X+Y)/2, where X is the earlier day when the number of survivors is N/2, and Y is the earliest day when the number of survivors (N/2)-1. If N is odd, the MedST (days) is X.

D. Computation of MedST from Mortality Distribution

If the total number of animals including survivors (N) is even, the MedST (days) (X+Y)/2, where X is the earliest day when the cumulative number of deaths is N/2, and Y is the earliest day when the cumulative number of deaths is (N/2)+1. If N is odd, the MedST (days) is X. “Cures” and “no-takes” in systems evaluated by MedST are based upon the day of evaluation. On the day of evaluation any survivor not considered a “no-take” is recorded as a “cure.” Survivors on day of evaluation are recorded as “cures” or “no-takes,” but not eliminated from the calculation.

E. Calculation of Approximate Tumor Weight from Measurement of Tumor Diameters with Vernier Calipers

The use of diameter measurements (with Vernier calipers) for estimating treatment effectiveness on local tumor size permits retention of the animals for lifespan observations. When the tumor is implanted sc, tumor weight is estimated from tumor diameter measurements as follows. The resultant local tumor is considered a prolate ellipsoid with one long axis and two short axes. The two short axes are assumed to be equal. The longest diameter (length) and the shortest diameter (width) are measured with Vernier calipers. Assuming specific gravity is approximately 1.0, and Pi is about 3, the mass (in mg) is calculated by multiplying the length of the tumor by the width squared and dividing the product by two. Thus,

$$Tumor weight ( mg ) = \frac{length ( mm ) \times ( width [ mm ] ) 2}{2} or \frac{L \times ( W ) 2}{2}$$

The reporting of tumor weights calculated in this way is acceptable inasmuch as the assumptions result in as much accuracy as the experimental method warrants.

### F. Calculation of Tumor Diameters

The effects of a drug on the local tumor diameter may be reported directly as tumor diameters without conversion to tumor weight. To assess tumor inhibition by comparing the tumor diameters of treated animals with the tumor diameters of control animals, the three diameters of a tumor are averaged (the long axis and the two short axes). A tumor diameter T/C of 75% or less indicates activity and a T/C of 75% is approximately equivalent to a tumor weight T/C of 42%.

G. Calculation of Mean Tumor Weight from Individual Excised Tumors

The mean tumor weight is defined as the sum of the weights of individual excised tumors divided by the number of tumors. This calculation is modified according to the rules listed below regarding “no-takes.” Small tumors weighing 39 mg or less in control mice or 99 mg or less in control rats, are regarded as “no-takes” and eliminated from the computations. In treated groups, such tumors are defined as “no-takes” or as true drug inhibitions according to the following rules:

| Percent of | | |
|---|---|---|
| small tumors | | |
| in treated | Percent of “no-takes” | |
| group | in control group | Action |
| ≦7 | Any percent | no-take; not used in calculations |
| 18-39 | <10 | drug inhibition; use in calculations |
| | ≧10 | no-takes; not used in calculations |
| ≧40 | <15 | drug inhibition; use in calculations |
| | ≧15 | Code all nontoxic tests “33” |

Positive control compounds are not considered to have “no-takes” regardless of the number of “no-takes” in the control group. Thus, the tumor weights of all surviving animals are used in the calculation of T/C for the positive control (T/C defined above) SDs of the mean control tumor weight are computed the factors in a table designed to estimate SD using the estimating factor for SD given the range (difference between highest and lowest observation) (Biometrik Tables for Statisticians Pearson E S & Hartley H G eds. Cambridge Press, vol. 1, table 22, p. 165).

### II. Specific Tumor Models

### A. Lymphoid Leukemia L1210

Summary: Ascitic fluid from donor mouse is transferred into recipient BDF1 or CDF1 mice. Treatment begins 24 hours after implant. Results are expressed as a percentage of control survival time. The key parameter is mean survival time. Origin of tumor line: induced in 1948 in spleen and lymph nodes of mice by painting skin with MCA (J Natl Cancer Inst. 13:1328 (1953)).

| | |
|---|---|
| Animals | One sex used for all test and control animals in one |
| | experiment. |
| Tumor Transfer | Inject ip, 0.1 ml of diluted ascitic fluid containing |
| | 10⁵ cells |
| Propagation | DBA/2 mice (or BDF1 or CDF1 for one generation). |
| Time of Transfer | Day 6 or 7 |
| Testing | BDF1 (C57BL/6 × DBA/2) or CDF1 (BALB/c × |
| | DBA/2) |
| Time of Transfer | Day 6 or 7 |
| Weight | Within a 3-g range, minimum weight of 18 g for |
| | males and 17 g for females. |
| Exp Size (n) | 6/group; No. of control groups varies according to |
| | number of test groups. |

### Testing Schedule

| DAY | PROCEDURE |
|---|---|
| 0 | Implant tumor. Prepare materials. Run positive control in every |
| | odd-numbered experiment. Record survivors daily. |
| 1 | Weigh and randomize animals. Begin treatment with therapeutic |
| | composition. Typically, mice receive 1 μg of the test |
| | composition in 0.5 ml saline. Controls receive saline alone. |
| | Treatment is one dose/week. Any surviving mice are sacrificed |
| | after 4 wks of therapy. |
| 5 | Weigh animals and record. |
| 20 | If there are no survivors except those treated with positive control |
| | compound, evaluate |
| 30 | Kill all survivors and evaluate experiment. |

Quality Control: Acceptable control survival time is 8-10 days. Positive control compound is 5-fluorouracil; single dose is 200 mg/kg/injection, intermittent dose is 60 mg/kg/injection, and chronic dose is 20 mg/kg/injection. Ratio of tumor to control (T/C) lower limit for positive control compound is 135%.
Evaluation: Compute mean animal weight on Days 1 and 5, and at the completion of testing compute T/C for all test groups with >65% survivors on Day 5. A T/C value 85% indicates a toxic test. An initial T/C 125% is considered necessary to demonstrate activity. A reproduced T/C 125% is considered worthy of further study. For confirmed activity a composition should have two multi-dose assays that produce a T/C 125%.

### B. Lymphocytic Leukemia P388

Summary: Ascitic fluid from donor mouse is implanted in recipient BDF1 or CDF1 mice. Treatment begins 24 hours after implant. Results are expressed as a percentage of control survival time. The key parameter is MedST. Origin of tumor line: induced in 1955 in a DBA/2 mouse by painting with MCA (Scientific Proceedings, Pathologists and Bacteriologists 33:603 (1957)).

| | |
|---|---|
| Animals | One sex used for all test and control animals in one |
| | experiment. |
| Tumor Transfer | Inject ip, 0.1 ml of diluted ascitic fluid containing |
| | 10⁶ cells |
| Propagation | DBA/2 mice (or BDF1 or CDF1 for one generation). |
| Time of Transfer | Day 7 |
| Testing | BDF1 (C57BL/6 × DBA/2) or CDF1 (BALB/c × |
| | DBA/2) |
| Time of Transfer | Day 6 or 7 |
| Weight | Within a 3-g range, minimum weight of 18 g for |
| | males and 17 g for females. |
| Exp Size (n) | 6/group; No. of control groups varies according to |
| | number of test groups. |

### Testing Schedule

| DAY | PROCEDURE |
|---|---|
| 0 | Implant tumor. Prepare materials. Run positive control in every |
| | odd-numbered experiment. Record survivors daily. |
| 1 | Weigh and randomize animals. Begin treatment with therapeutic |
| | composition. Typically, mice receive 1 μg of the test |
| | composition in 0.5 ml saline. Controls receive saline alone. |
| | Treatment is one dose/week. Any surviving mice are sacrificed |
| | after 4 wks of therapy. |
| 5 | Weigh animals and record. |
| 20 | If there are no survivors except those treated with positive control |
| | compound, evaluate |
| 30 | Kill all survivors and evaluate experiment. |

Acceptable MedST is 9-14 days. Positive control compound is 5-fluorouracil: single dose is 200 mg/kg/injection, intermittent dose is 60 mg/kg/injection, and chronic dose is 20 mg/kg/injection. T/C lower limit for positive control compound is 135% Check control deaths, no takes, etc.
Quality Control: Acceptable MedST is 9-14 days. Positive control compound is 5-fluorouracil: single dose is 200 mg/kg/injection, intermittent dose is 60 mg/kg/injection, and chronic dose is 20 mg/kg/injection. T/C lower limit for positive control compound is 135%. Check control deaths, no takes, etc.
Evaluation: Compute mean animal weight on Days 1 and 5, and at the completion of testing compute T/C for all test groups with >65% survivors on Day 5. A T/C value of 85% indicates a toxic test. An initial T/C of 125% is considered necessary to demonstrate activity. A reproduced T/C 125% is considered worthy of further study. For confirmed activity a composition should have two multi-dose assays that produce a T/C 125%.

### C. Melanotic Melanoma B16

Summary: Tumor homogenate is implanted ip or sc in BDF1 mice. Treatment begins 24 hours after either ip or sc implant or is delayed until an sc tumor of specified size (usually approximately 400 mg) can be palpated. Results expressed as a percentage of control survival time. The key parameter is mean survival time. Origin of tumor line: arose spontaneously in 1954 on the skin at the base of the ear in a C57BL/6 mouse (Handbook on Genetically Standardized Jax Mice. Jackson Memorial Laboratory, Bar Harbor, Me., 1962. See also Ann NY Acad Sci 100, Parts 1 and 2, (1963)).

| | |
|---|---|
| Animals | One sex used for all test and control animals in one |
| | experiment. |
| Propagation Strain | C57BL/6 mice |
| Tumor Transfer | Implant fragment sc by trochar or 12-g needle or |
| | tumor homogenate* every 10-14 days into axillary |
| | region with puncture in inguinal region. |
| Testing Strain | BDF1 (C57BL/6 × DBA/2) |
| Time of Transfer | Excise sc tumor on Day 10-14 from donor mice |
| | and implant as above |
| Weight | Within a 3-g range, minimum weight of 18 g for |
| | males and 17 g for females. |
| Exp Size (n) | 10/group; No. of control groups varies according to |
| | number of test groups. |

*Tumor homogenate: Mix 1 g or tumor with 10 ml of cold balanced salt solution, homogenize, and implant 0.5 ml of tumor homogenate ip or sc. Fragment: A 25-mg fragment may be implanted sc.

### Testing Schedule

| DAY | PROCEDURE |
|---|---|
| 0 | Implant tumor. Prepare materials. Run positive control in every |
| | odd-numbered experiment. Record survivors daily. |
| 1 | Weigh and randomize animals. Begin treatment with therapeutic |
| | composition. Typically, mice receive 1 μg of the test composition |
| | in 0.5 ml saline. Controls receive saline alone. |
| | Treatment is one dose/week. Any surviving mice are sacrificed |
| | after 8 wks of therapy. |
| 5 | Weigh animals and record. |
| 60 | Kill all survivors and evaluate experiment. |

Quality Control: Acceptable control survival time is 14-22 days. Positive control compound is 5-fluorouracil: single dose is 200 mg/kg/injection, intermittent dose is 60 mg/kg/injection, and chronic dose is 20 mg/kg/injection. T/C lower limit for positive control compound is 135% Check control deaths, no takes, etc.
Evaluation: Compute mean animal weight on Days 1 and 5, and at the completion of testing compute T/C for all test groups with >65% survivors on Day 5. A T/C value of 85% indicates a toxic test. An initial T/C of 125% is considered necessary to demonstrate activity. A reproduced T/C 125% is considered worthy of further study. For confirmed activity a composition should have two multi-dose assays that produce a T/C 125%.
Metastasis after IV Injection of Tumor Cells

10⁵ B16 melanoma cells in 0.3 ml saline are injected intravenously in C57BL/6 mice. The mice are treated intravenously with 1 g of the composition being tested in 0.5 ml saline. Controls receive saline alone. Mice sacrificed after 4 weeks of therapy, the lungs are removed and metastases are enumerated.

### C. 3LL Lewis Lung Carcinoma

Summary: Tumor may be implanted sc as a 2-4 mm fragment, or im as a 2×10⁶-cell inoculum. Treatment begins 24 hours after implant or is delayed until a tumor of specified size (usually approximately 400 mg) can be palpated. Origin of tumor line: arose spontaneously in 1951 as carcinoma of the lung in a C57BL/6 mouse Cancer Res 15:39, (1955)). See also Malave I et al., J. Natl. Canc. Inst. 62:83-88 (1979).

| | |
|---|---|
| Animals | One sex used for all test and control animals in |
| | one experiment. |
| Propagation Strain | C57BL/6 mice |
| Tumor Transfer | Inject cells im in hind leg or implant fragment sc |
| | in axillary region with puncture in inguinal region. |
| | Transfer on day 12-14 |
| Testing Strain | BDF1 (C57BL/6 × DBA/2) or C3H mice |
| Time of Transfer | Same as above |
| Weight | Within a 3-g range, minimum weight of 18 g for |
| | males and 17 g for females. |
| Exp Size (n) | 6/group for sc implant, or 10/group for im implant.; |
| | No. of control groups varies according to number |
| | of test groups. |

### Testing Schedule

| DAY | PROCEDURE |
|---|---|
| 0 | Implant tumor. Prepare materials. Run positive control in |
| | every odd-numbered experiment. Record survivors daily. |
| 1 | Weigh and randomize animals. Begin treatment with |
| | therapeutic composition. Typically, mice receive 1 μg of the |
| | test composition in 0.5 ml saline. Controls receive saline |
| | alone. Treatment is one dose/week. Any surviving mice are |
| | sacrificed after 4 wks of therapy. |
| 5 | Weigh animals and record. |
| Final day | Kill all survivors and evaluate experiment. |

Quality Control: Acceptable im tumor weight on Day 12 is 500-2500 mg. Acceptable im tumor MedST is 18-28 days. Positive control compound is cyclophosphamide: 20 mg/kg/injection, qd, Days 1-11. Check control deaths, no takes, etc.
Evaluation: Compute mean animal weight when appropriate, and at the completion of testing compute T/C for all test groups. When the parameter is tumor weight, a reproducible T/C of 42% is considered necessary to demonstrate activity. When the parameter is survival time, a reproducible T/C of 125% is considered necessary to demonstrate activity. For confirmed activity a composition must have two multi-dose assays

### D. 3LL Lewis Lung Carcinoma Metastasis Model

This model has been utilized by a number of investigators. See, for example, Gorelik, E. et al., J. Natl. Canc. Inst. 65:1257-1264 (1980); Gorelik, E. et al., Rec. Results Canc. Res. 75:20-28 (1980); Isakov, N. et al., Invasion Metas. 2:12-32 (1982) Talmadge J E et al., J. Nat'l. Canc. Inst. 69:975-980 (1982); Hilgard, P. et al., Br. J. Cancer 35:78-86 (1977)).

Mice: male C57BL/6 mice, 2-3 months old. Tumor: The 3LL Lewis Lung Carcinoma was maintained by sc transfers in C57BL/6 mice. Following sc, im or intra-footpad transplantation, this tumor produces metastases, preferentially in the lungs. Single-cell suspensions are prepared from solid tumors by treating minced tumor tissue with a solution of 0.3% trypsin. Cells are washed 3 times with PBS (pH 7.4) and suspended in PBS. Viability of the 3LL cells prepared in this way is generally about 95-99% (by trypan blue dye exclusion). Viable tumor cells (3×10⁴-5×10⁶) suspended in 0.05 ml PBS are injected into the right hind foot pads of C57BL/6 mice. The day of tumor appearance and the diameters of established tumors are measured by caliper every two days.

In experiments involving tumor excision, mice with tumors 8-10 mm in diameter are divided into two groups. In one group, legs with tumors are amputated after ligation above the knee joints. Mice in the second group are left intact as nonamputated tumor-bearing controls. Amputation of a tumor-free leg in a tumor-bearing mouse has no known effect on subsequent metastasis, ruling out possible effects of anesthesia, stress or surgery. Surgery is performed under Nembutal anesthesia (60 mg veterinary Nembutal per kg body weight).

### Determination of Metastasis Spread and Growth

Mice are killed 10-14 days after amputation. Lungs are removed and weighed. Lungs are fixed in Bouin's solution and the number of visible metastases is recorded. The diameters of the metastases are also measured using a binocular stereoscope equipped with a micrometer-containing ocular under 8× magnification. On the basis of the recorded diameters, it is possible to calculate the volume of each metastasis. To determine the total volume of metastases per lung, the mean number of visible metastases is multiplied by the mean volume of metastases. To further determine metastatic growth, it is possible to measure incorporation of ^{125I}dUrd into lung cells (Thakur M L et al., J. Lab. Clin. Med. 89:217-228 (1977)). Ten days following tumor amputation, 25 mg of ¹²⁵IdUrd is inoculated into the peritoneums of tumor-bearing (and, if used, tumor-resected mice. After 30 min, mice are given 1 mCi of ¹²⁵IdUrd. One day later, lungs and spleens are removed and weighed, and a degree of ¹²⁵IdUrd incorporation is measured using a gamma counter.

Statistics: Values representing the incidence of metastases and their growth in the lungs of tumor-bearing mice are not normally distributed. Therefore, non-parametric statistics such as the Mann-Whitney U-Test may be used for analysis.
Study of this model by Gorelik et al. (1980, supra) showed that the size of the tumor cell inoculum determined the extent of metastatic growth. The rate of metastasis in the lungs of operated mice was different from primary tumor-bearing mice. Thus in the lungs of mice in which the primary tumor had been induced by inoculation of large doses of 3LL cells (1-5×10⁶) followed by surgical removal, the number of metastases was lower than that in nonoperated tumor-bearing mice, though the volume of metastases was higher than in the nonoperated controls. Using ¹²⁵IdUrd incorporation as a measure of lung metastasis, no significant differences were found between the lungs of tumor-excised mice and tumor-bearing mice originally inoculated with 10⁶ 3 LL cells. Amputation of tumors produced following inoculation of 10⁵ tumor cells dramatically accelerated metastatic growth. These results were in accord with the survival of mice after excision of local tumors. The phenomenon of acceleration of metastatic growth following excision of local tumors had been observed by other investigators. The growth rate and incidence of pulmonary metastasis were highest in mice inoculated with the lowest doses (3×10⁴-10⁵ of tumor cells) and characterized also by the longest latency periods before local tumor appearance. Immunosuppression accelerated metastatic growth, though nonimmunologic mechanisms participate in the control exerted by the local tumor on lung metastasis development. These observations have implications for the prognosis of patients who undergo cancer surgery.

### E. Walker Carcinosarcoma 256

Summary: Tumor may be implanted sc in the axillary region as a 2-6 mm fragment im in the thigh as a 0.2-ml inoculum of tumor homogenate containing 10⁶ viable cells, or ip as a 0.1-ml suspension containing 10⁶ viable cells. Origin of tumor line: arose spontaneously in 1928 in the region of the mammary gland of a pregnant albino rat (J Natl Cancer Inst 13:1356, (1953)).

| | |
|---|---|
| Animals | One sex used for all test and control animals in one |
| | experiment. |
| Propagation | Random-bred albino Sprague-Dawley rats |
| Strain | S.C. fragment implant is by trochar or 12-g needle |
| Tumor Transfer | into axillary region with puncture in inguinal area. |
| | I.m. implant is with 0.2 ml of tumor homogenate |
| | (containing 10⁶ viable cells) into the thigh. I.p. implant |
| | is with 0.1 ml suspension (containing 10⁶ viable cells) |
| | Day 7 for im or ip implant; Days 11-13 for sc implant |
| Testing Strain | Fischer 344 rats or random-bred albino rats |
| Time of Transfer | Same as above |
| Weight | 50-70 g (maximum of 10-g weight range within each |
| | experiment) |
| Exp Size (n) | 6/roup; No. of control groups varies according to |
| | number of test groups. |

| Test | Prepare drug | Administer | Weigh animals | Evaluate on |
|---|---|---|---|---|
| system | on day: | drug on days: | on days | days |
| 5WA16 | 2 | 3-6 | 3 and 7 | 7 |
| 5WA12 | 0 | 1-5 | 1 and 5 | 10-14 |
| 5WA31 | 0 | 1-9 | 1 and 5 | 30 |

In addition the following general schedule is followed

| DAY | PROCEDURE |
|---|---|
| 0 | Implant tumor. Prepare materials. Run positive control in every |
| | odd-numbered experiment. Record survivors daily. |
| 1 | Weigh and randomize animals. Begin treatment with therapeutic |
| | composition. Typically, mice receive 1 μg of the test |
| | composition in 0.5 ml saline. Controls receive saline alone. |
| | Treatment is one dose/week. Any surviving mice are sacrificed |
| | after 4 wks of therapy. |
| Final | Kill all survivors and evaluate experiment. |
| day | |

Quality Control: Acceptable i.m. tumor weight or survival time for the above three test systems are: 5WA16: 3-12 g; 5WA12: 3-12 g; 5WA31 or 5WA21: 5-9 days.
Evaluation: Compute mean animal weight when appropriate, and at the completion of testing compute T/C for all test groups. When the parameter is tumor weight, a reproducible T/C 42% is considered necessary to demonstrate activity. When the parameter is survival time, a reproducible T/C 125% is considered necessary to demonstrate activity. For confirmed activity

### F. A20 Lymphoma

10⁶ murine A20 lymphoma cells in 0.3 ml saline are injected subcutaneously in Balb/c mice. Tumor growth is monitored daily by physical measurement of tumor size and calculation of total tumor volume. After 4 weeks of therapy the mice are sacrificed.

### Formulations

Compositions of the invention comprising modified cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the modified cells utilized in practicing the present invention in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added.

Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the genetically modified immunoresponsive cells or their progenitors.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the genetically modified immunoresponsive cells as described in the present invention. This will present no problem to those skilled in chemical and pharmaceutical principles, or problems can be readily avoided by reference to standard texts or by simple experiments (not involving undue experimentation), from this disclosure and the documents cited herein. One consideration concerning the therapeutic use of modified cells of the invention is the quantity of cells necessary to achieve an optimal effect. The quantity of cells to be administered will vary for the subject being treated. In a one embodiment, between 10⁴ to 10¹⁰, between 10⁵ to 10⁹, or between 10⁶ and 10⁸ modified cells of the invention are administered to a human subject. In preferred embodiments, at least about 1×10⁸2×10⁸, 3×10⁸, 4×10⁸, and 5×10⁸ modified cells of the invention are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art. The skilled artisan can readily determine the amount of cells and optional additives, vehicles, and/or carrier in compositions and to be administered in methods of the invention. Typically, any additives (in addition to the active stem cell(s) and/or agent(s)) are present in an amount of 0.001 to 50% (weight) solution in phosphate buffered saline, and the active ingredient is present in the order of micrograms to milligrams, such as about 0.0001 to about 5 wt %, preferably about 0.0001 to about 1 wt %, still more preferably about 0.0001 to about 0.05 wt % or about 0.001 to about 20 wt %, preferably about 0.01 to about 10 wt %, and still more preferably about 0.05 to about 5 wt %. Of course, for any composition to be administered to an animal or human, and for any particular method of administration, it is preferred to determine therefore: toxicity, such as by determining therefore toxicity such as by determining lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### Methods of Treatment

Provided herein are methods for treating neoplasia in a subject. The methods comprise administering a cells of the invention in an amount effective to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

An “effective amount” (or, “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the antigen-binding fragment administered.

For therapy using T cells, cell doses in the range of 10⁹ are typically infused. Upon administration of the genetically modified cells into the host and subsequent differentiation, T cells are induced that are specifically directed against the specific antigen. The modified cells can be administered by any method known in the art including, but not limited to, intravenous, subcutaneous, intranodal, intratumoral, intrathecal, intrapleural, intraperitoneal and directly to the thymus.

The invention provides methods for increasing an immune response in a subject in need thereof. In one embodiment, the invention provides methods for treating or preventing a neoplasia in a subject. The invention provides therapies that are particularly useful for the treatment of subjects having cancer, or metastatic cancer that is not amenable to conventional therapeutic interventions. Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition embodied in this invention is administered to these subjects to elicit an anti-tumor response, with the objective of palliating their condition. Ideally, reduction in tumor mass occurs as a result, but any clinical improvement constitutes a benefit. Clinical improvement includes decreased risk or rate of progression or reduction in pathological consequences of the tumor.

A second group of suitable subjects is known in the art as the “adjuvant group.” These are individuals who have had a history of neoplasia, but have been responsive to another mode of therapy. The prior therapy can have included (but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasia. Features typical of high-risk subgroups are those in which the tumor has invaded neighboring tissues, or who show involvement of lymph nodes.

Another group have a genetic predisposition to neoplasia but have not yet evidenced clinical signs of neoplasia. For instance, women testing positive for a genetic mutation associated with breast cancer, but still of childbearing age, can wish to receive one or more of the antigen-binding fragments described herein in treatment prophylactically to prevent the occurrence of neoplasia until it is suitable to perform preventive surgery.

Human neoplasia subjects having any of the following neoplasias: glioblastoma, melanoma, neuroblastoma, adenocarcinoma, glioma, soft tissue sarcoma, and various carcinomas (including prostate and small cell lung cancer) are especially appropriate subjects. Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolarcarcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarci-noma, medullary carcinoma, bronchogenic carcinoma, renal cell carcinoma, bile duct carcinoma, choriocarcinoma, seminoma, embryonal carcinoma, Wilms’ tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of pro static adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. [0131] The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Accordingly, the invention provides a method of treating or preventing a neoplasia in a subject, the method comprising administering an effective amount of an immunoresponsive cell comprising a receptor ligand or antibody that binds a tumor receptor or tumor antigen and a vector encoding a SAg and an MHCII ligand that engages the SAg and presents it to the TCR vβ region. In one embodiment, the neoplasia is selected from the group consisting of prostate cancer, colon cancer, breast cancer, and glioblastoma. In another embodiment, the tumor receptor is the EGF receptor and the tumor antigen is prostate-specific membrane antigen, CD19, NY-ESO-1, WT-1 or hTERT. As a consequence of constitutive surface expression of SAg and MHCII, adoptively transferred human T or NK cells are endowed with augmented proliferative, cytolytic, and survival capacities in an intrinsically poorly immunogenic tumor environment devoid of co-stimulatory ligands. Furthermore, subsequent to their localization to tumor and their proliferation, co-stimulatory ligand-expressing T cells turn the tumor site into a highly conductive environment for a wide range of immune cells involved in the physiological anti-tumor response (tumor infiltrating lymphocytes, NK-, NKT-cells, dendritic cells, and macrophages).

### Self Stimulating Cytotoxic T Lymphocytes

The present invention provides self stimulating immunoresponsive T cells and Natural Killer (NK) cells, expressing a superantigen, at least one of an antigen-recognizing receptor, a MHCII motif that binds said superantigen linked to the CD3 zeta signaling domain and methods of use for the treatment of neoplasia. The invention provides a tumor antigen-specific T cell expressing a vector encoding a superantigen polypeptide or superantigen homologue selected from the group consisting of SEA to SEU, one or more MHCII polypeptides comprising alpha and/or beta chains or functional fragments thereof, a tumor specific receptor that binds to a tumor receptor ligand or and the intracellular CD3 signaling domain zeta chain that activates the T cell or cytotoxic T cell. An fv single chain antibody fragment that binds to a tumor antigen may be used in place of the tumor receptor for tumor targeting. The vector incorporating this construct is a retroviral vector (e.g., gamma-retroviral or lentiviral); it may also be non-viral. The neoplasia is selected from the any one or more of prostate cancer, colon cancer, breast cancer, and glioblastoma. In another embodiment, the tumor receptor recognizes a tumor cell receptor ligand such the EGF receptor ligand expressed on tumor cells.

In another aspect, the invention provides a method of treating a neoplasia in a subject in need thereof, the method comprising administering to the subject a therapeutically effective amount of a T cell comprising a tumor specific receptor ligand, an intact MHCII molecule or an MHCII alpha or beta chain that engages a superantigen and a superantigen or superantigen homologue or superantigen fusion protein and the CD3 signaling domain zeta where at least one of said superantigen or superantigen homologue or superantigen fusion protein is selected from any one or more of SEA to SEU, thereby treating cancer in the subject. In one embodiment, the selected superantigen is SEG. In another embodiment, the tumor specific receptor ligand and the CD3 zeta domain are expressed on the T lymphocyte and the superantigen and MHCII molecule are inducibly expressed on the surface of the T lymphocyte after engagement of the tumor receptor ligand with its cognate receptor on the T cell. In another aspect, the invention provides a pharmaceutical composition comprising an effective amount of an immunoresponsive cell of any previous aspect in a pharmaceutically acceptable excipient.

In another aspect, the invention provides a pharmaceutical composition for the treatment of a neoplasia comprising an effective amount of a T cell expressing a tumor specific receptor ligand and a superantigen and MHCII molecules such as alpha and/or beta chains of any previous aspect in a pharmaceutically acceptable excipient.

In one embodiment, the composition further comprise a cytokine selected from any one or more of IL-2, IL-3, IL-6, IL-11, 1L7, IL12, IL15, IL21, granulocyte macrophage colony stimulating factor, alpha, beta or gamma interferon.

In other embodiments of the previous aspects, the co-stimulatory ligand is constitutively and/or inducibly expressed. In various embodiments of any previous aspect, the cell is selected from any one or more of a T cell, a cytotoxic T lymphocyte (CTL). In still other embodiments of the previous aspects, the antigen or tumor receptor, e.g., any one or more of prostate-specific membrane antigen (PSMA), Carcinoembryonic Antigen (CEA), IL13R alpha, her-2, CD19, NY-ESO-1, HIV-1 Gag, Lewis Y, Mart-1, gp100, tyrosinase, WT-1, hTERT, mesothelin.

In another embodiments of the previous aspects, the T cell expresses a recombinant and/or an endogenous tumor specific antibody, single chain fv or Fab fragment. In other embodiments, the superantigen is SEA-U, or the SPEA-C and their functional homologues as defined herein. In still other embodiments of the previous aspects, the MHCII molecule is an alpha or beta chain or functional fragment(s) thereof or a combination thereof. In still other embodiments of the previous aspects, the immunoresponsive cell expresses at least one alpha or beta chain or a combination thereof. In still other embodiments of the previous aspects, a tumor antigen recognizing complex and/or superantitgens are constitutively or inducibly expressed on the surface of the T cell. In still other embodiments of the previous aspects the superantigen and MHC molecules are expressed in a retroviral vector. In another embodiment, the tumor receptor is the EGF receptor. Enhanced EGFR expression has been found in a wide variety of malignancies, including esophageal, lung, thyroid, gastric, bladder, renal, prostate, ovarian, endometrial and breast. One mucinous adenocarcinoma was shown to have an extreme elevation in levels of EGF receptors, corresponding to a 320-fold increase over normal mucosa. In still other embodiments, the cell expresses a recombinant or an endogenous receptor for the antigen. In various embodiments, the intracellular signaling domain is a zeta-chain signaling domain. In other embodiments, one T cell activating signal is via engagement of the engineered tumor receptor ligand on the T cell surface interfacing with its cognate antigen on the tumor cell. Upon this interaction, the CD3 zeta signaling domain operably linked to the tumor receptor ligand is activated and induces T cell synthesis of the SAg and MHII molecules. The latter two molecules are positioned on the cell surface and form a quadrimoleular complex with constitutive with their cognate TCR vβ receptors and CD28 in close proximity on the same cell surface. This results in robust T cell proliferation and generation of cytotoxics granzyme and perforin which are transferred via the T cell synapse with the tumor cell and kill the tumor cell.

### DEFINITIONS

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (e.g., Wahl, G. M. and S. L. Berger Methods Enzymol. 152:399 (1987); Kimmel, A. R. Methods Enzymol. 152:507 (1987)).

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

The term “constitutive expression” as used herein refers to expression under all physiological conditions.

The term “tumor antigen receptor” as used herein refers to a tumor antigen-binding domain that is fused to an intracellular signaling domain capable of activating T cells. Most commonly, the tumor antigen receptor's extracellular binding domain is derived from a murine or humanized tumor receptor ligand or monoclonal antibody. The term “tumor receptor” as used herein refers to a domain capable of binding a constitutive receptor positioned on the tumor cell surface particularly its extracellular domain. By “effective amount” is meant an amount sufficient to arrest, ameliorate, or inhibit the continued proliferation, growth, or metastasis (e.g., invasion, or migration) of a neoplasia.

By “immunoresponsive cell” is meant a cell that functions in an immune response or a progenitor, or progeny thereof.

By “neoplasia” is meant a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasias can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasias include cancers, such as sarcomas, carcinomas, or plasmacytomas (malignant tumor of the plasma cells). Illustrative neoplasms for which the invention can be used include, but are not limited to leukemias (e.g., acute leukemia, acute lymphocytic leukemia, acute myelocytic leukemia a, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). In one embodiment, screening methods of the invention identify compositions that are useful for treating prostate, lung and breast cancer.

The term “tumor antigen” as used herein refers to any polypeptide expressed by a tumor that is capable of inducing an immune response.

### DETAILED DESCRIPTION

The present invention contemplates that peripheral-blood T cells are useful for transduction with a self-inactivating lentiviral vector carrying genes to produce self activating cytotoxic T cells with tumor specificity. The invention is based on the limitation to effective anti-tumor therapy with T cell is paucity of costimulation within the tumor microenvironment. It is now appreciated that the activity of T cells depends both on proper engagement of the TCR with appropriate affinity ligands and on additional signals collectively referred to as ‘costimulatory signals. These costimulatory signals are delivered by specific ligands on antigen-presenting cells binding to cognate costimulatory receptors on the T cell. One of the checks on specificity during the adaptive immune response is that costimulatory signals affect T cell activation only when the TCR signal is simultaneously delivered. The most important costimulatory ligands fall into two general families. One is the B7 family, and the other constitutes a limited subset of tumor necrosis factor (TNF) family members. The canonical pair of B7 family costimulatory members are CD80 and CD86, each of which binds to the CD28 costimulatory receptor on T cells, whereas there are four or five TNF family costimulatory ligands that interact with specific cognate TNF receptor family receptors on the T cell. The present invention exploits the remarkable T cell stimulating properties of superantigens along with the recently described discovery of CD28 binding domains on several superantigens that regulate the strength and quality of the T cell response (Arad et al., PLoS Biol. 2011 September; 9 (9):e1001149. doi: 10.1371; Kaempfer et al., Toxins 5:1531-42 (2013)). The key elements of the inventive construct used to transduce cytotoxic T lymphocytes or NK cells include a superantigen preferably one with minimal cytokine inducing activity such as SEG and the extracellular domain of its cognate MHCII receptor. The latter may be the extracellular domain or the alpha or beta chain or functional fragment thereof for which the SAg has particular affinity. The third element is a tumor targeting molecule which may include a tumor specific receptor ligand or a tumor specific single chain fv antibody fragment. The entire construct is linked to the CD3 zeta signaling molecule. The latter ensures that T cell autocostimulation and T cell activation only occurs when the T cell tumor receptor ligand is first triggered by engagement of the T cells with a tumor cell expressing the cognate receptor or tumor antigen targeted by the receptor ligand or T-body. Such engagement sets in motion surface expression of the SAg and the extracellular domain of is cognate MHCII alpha and/or beta chain. In principle, SAg-MHCII expression on the cell surface leads to immediate high affinity binding of the SAg to its cognate vβ TCR binding site(s) and CD28 situated on the surface of the T lymphocyte. This results in profound CD8+ T cell proliferation at the tumor site release of granzyme/perforin cytotoxins and consequent tumor cell apoptosis. In the inventive construct, a single MHC alpha or beta chain may be used in place of the entire molecule since many SAg show affinity only for one or the other of these chains. SEG and SEB for example bind only to the alpha chain whereas SEA and SEC bind with high affinity to the beta chain. Formation of the MHCII-SAg complex on the T cell surface then activates adjacent vβ regions of the TCR which are constitutively expressed on the T cell surface. This activation leads to robust T cell autostimulation.

T cell proliferation reaches a peak shortly after T SAg activation. Such autostimulation is facilitated by SAg binding to the CD28 receptor constitutively expressed on the surface of the T cell. Such SAg-CD28 interaction controls the degree of cytokine activation by the SAg. In the case of SEG, such interaction results in a significant reduction of SAg activated cytokines such as IFN-γ and TNF-α compared to classical superantigens such as SEA. These cytotoxic and tumor specific T cells are introduced parenterally into the host. To avoid seroreactivity by neutralizing antibodies, SEG or other superantigens of the enterotoxin growth cluster (SEI, SEM, SEN, SEO) to which <5% of humans make such neutralizing antibodies are preferred. Moreover, according to the claimed invention, the SAg is not expressed on the surface of the T cell until its tumor targeting receptor ligand engages it cognate tumor receptor on the tumor cell. Such a tumor targeting motif ensures that T cell superantigen is not exposed to seroreactive neutralizing antibodies present in the circulation. The cytotoxic T cells activated after interacting with the tumor continue to proliferate in the tumor microenvironment resulting in elimination of tumor at multiple tumor sites in the host.

Any superantigen is useful in this invention but those expressing intrinsic ligands for MHCII and TCR vβ regions and the costimulatory CD28 receptor are preferred. These include the staphylococcal enterotoxin A-U as given herein. In this context SEG stands out as the preferred superantigen because calorimetric and SPR analysis showed that SEG has an affinity for mVβ8.2 40 to 100-fold higher than that reported for other members of SEB subfamily, and the highest reported for a wild type SAg-TCR couple. Its affinity for MHCII is confined to the alpha chain rather than both alpha and beta chain characteristic of several other superantigens. Importantly, it also has a distinctively truncated TH-1 cytokine profile and an extremely low incidence of naturally occurring neutralizing antibodies in human sera. After T cell activation, SEG also initiates more rapid T proliferation than other SAgs which has advantages when such T cells interface with their tumor cell targets.

Human class II MHC molecules are of 3 major isotypes, HLA-DR, HLA-DP, and HLA-DQ each of which consists of an alpha and a beta chain. Both the alpha and beta chains of HLA-DP and HLA-DQ are polymorphic, whereas HLA-DR alpha (HLA-DRA) is invariant and HLA-DR beta is polymorphic. Thus, differences in the HLA-DR beta chain account for different peptide-binding motifs of HLA-DR molecules.

The MHCII alpha or beta chain in the instant invention can be exclusively HLA-DR or HLA-DQ or HLA-DP depending on the preferred chain a given SAg uses for maximal induction of T cell proliferation and differentiation. In some cases an HLA-DR or DQ alpha or beta chain may be used such as one that is associated with SAg overreactivity in disease states such as rheumatoid arthritis. The RA associated HLA-DQB1*03.02 and HLADQA1*03.02 genes (HLA-DQ8) are preferred. The amino acid nucleotide sequences of both these chains is provided below.

Human MHC class II HLA-DQ-beta (DQw3) GenBank: M16996.1 (SEQID NO:8)

| ORIGIN | |
|---|---|
| 23 bp upstream of BamHI site; chromosome 6p21.3. | |
| 1 | atgtcttgga aaaaggcttt gcggatcccc ggaggccttc gggtagcaac tgtgaccttg |
| 61 | atgctggcga tgctgagcac ctcggtggct gagggcagag actctcccga ggatttcgtg |
| 121 | taccagttta agggcatgtg ctacttcacc aacgggacgg agcgcgtgcg tcttgtgacc |
| 181 | agatacatct ataaccgaga ggagtacgca cgcttcgaca gcgacgtggg ggtgtaccgg |
| 241 | gcggtgacgc cgctggggcc gcctgacgcc gagtactgga acagccagaa ggaagtccta |
| 301 | gaggggaccc gggcggagtt ggacacggtg tgcagacaca actaccagtt ggagctccgc |
| 361 | acgaccttgc agcggcgagt ggagcccaca gtgaccatct ccccatccag gacagaggcc |
| 421 | ctcaaccacc acaacctgct ggtctgctca gtgacagatt tctatccagc ccagatcaaa |
| 481 | gtccggtggt ttcggaatga tcaggaggag acaaccggcg ttgtgtccac ccccctcatt |
| 541 | aggaacggtg actggacctt ccagatcctg gtgatgctgg aaatgactcc ccagcgtgga |
| 601 | gatgtctaca cctgccacgt ggagcacccc agcctccaga cccccatcac cgtggagtgg |
| 661 | cgggctcagt ctgaatctgc ccagagcaag atgctgagtg gcattggagg cttcgtgctg |
| 721 | gggctaatct tcctcgggct gggccttatt atccatcaca ggagtcagaa agggctcctg |
| 781 | cactga |

(SEQ ID NO: 9)
MSWKKALRIPGGLRVATVTLMLAMLSTSVAEGRDSPEDFVYQFK
GMCYFTNGTERVRLVTRYIYNREEYARFDSDVGVYRAVTPLGPPDAEYWNSQKEVLEG
TRAELDTVCRHNYQLELRTTLQRRVEPTVTISPSRTEALNHHNLLVCSVTDFYPAQIK
VRWFRNDQEETTGVVSTPLIRNGDWTFQILVMLEMTPQRGDVYTCHVEHPSLQTPITV
EWRAQSESAQSKMLSGIGGFVLGLIFLGLGLIIHHRSQKGLLH″

Homo sapiens HLA-DQA1 gene for MHC class II antigen, allele HLA-DQA1*03:01:01, exons 1-4 GenBank: HF572912.1 (SEQID NO:10)

| | |
|---|---|
| 1 | cttgtcttga ggccctcaca attgctctac agctcagaac agcaactgct gaggctgcct |
| 61 | tgggaagagg atgatcctaa acaaagctct gatgctgggg gccctcgccc tgaccaccgt |
| 121 | gatgagccct tgtggaggtg aagacattgt gggtgagtgc atgagtgagg aatgttctct |
| 181 | ggagctgaaa aacagtaaat tgaaggaaaa gagagaaagc gatttgcaga gaaattgtag |
| 241 | agatttccta agaccccttt cagtattaag agaattaaaa attatagctg ttcctccttc |
| 301 | aggaaaccag agccccaacc tactcttttt gttatgtatg cttttgtgtt cactaaggat |
| 361 | gctattctgt ttatattata ttcagtgaca acagcctgga ggtctctatg tcgttccgtc |
| 421 | atgattgcct caaaaattag tgaagtttcc atcagtggat aattttttat tattaaaaat |
| 481 | gtatgaagtg tcattctcaa atttccctga acaacttttg aagcttttcg tatgtctcct |
| 541 | gtagtagatc ttggggtcgt tccatcaatt atatactcta tagatattaa aaaagttgcc |
| 601 | cgtttctttc tctcagactt actcacattt ccacatggga actggcacag gtggggagtg |
| 661 | ggtaaaggag tccagcaggc tgaatgcctt caacaatcat tttaccacat ggtcctcact |
| 721 | tactctcagc tgcctcatat gtgtcacctc acaaataatc aaataaaatg ggcatgtagc |
| 781 | taagctttgt aaatagtgaa aacatggatg tcaattgttt ttacatattt ctattacagg |
| 841 | tatagcttca catttttctt tagcaaaata agggatcctt ttagtttaaa attgagaagt |
| 901 | agaaaaaatt ggtaaattaa atcattttat tctcaaatta tcaacccaaa ttacctgttc |
| 961 | ttcacctcat ctaataaagt cctataaaaa gaaaagtggg ccagacatgg tggctcatgc |
| 1021 | ctgtaatccc agcactttgg gaggccgaag caggaggatc atttgagcct gggagtttga |
| 1081 | gaccagcctg ggcaacatag caagacctca tctctaccaa aaaataaaat aaaaattagc |
| 1141 | caggtgtggt ggtgcatgcc tgtggtgcca gctactcaga aggctgcagt gggaggagca |
| 1201 | cttgagtcca ggaggtggaa gctgcagtga gccatgatgg caccactaca ctccagccag |
| 1261 | ggcaacagag agagactctg tctcaaaaag aaagaggaaa gaaagagaga aaggaaggaa |
| 1321 | agaaggaaag aaggaaggaa ggaaggagaa agggaaggga ggaaggaaaa aagaaagaaa |
| 1381 | gaaagaaaac ggaaggaagg aagcacagat taattatttg gtctctttgt ctcctctgcc |
| 1441 | tttgtcgtcc atctcttccc acctctcttc atgcattcct ttctccctct tccctttcag |
| 1501 | gatccatctc tgactccctg ctcctttata gagatggaca gtgagtttgt aaaacaaaag |
| 1561 | ttgaaaagtc agatagttaa aaggggaagt gaactggaag gtactctaaa ctttcacaac |
| 1621 | cttattaacc atggctgctc ccattctgat tttgttcagc agtggaagtt tcacccgctc |
| 1681 | ctccagagcg cttggcttct ttgttccaaa tttcctttct tcaacctcac accagagtgc |
| 1741 | cctggtcagg ctcagctcat ccattaggca caatgtgggc agtgcagggg accctccaga |
| 1801 | ctgtaaagcc acatgagaat gttttaactc cttttaaaat tataaaaaaa tgaaattgta |
| 1861 | gagcctaaga aaatgtttta acttttaatt cagcctagat tatattgtct ttataccaat |
| 1921 | tcagtcataa aatatagttt tccatatttt tatggaggaa ggcgtccaca caagcaagag |
| 1981 | tgcttggggc tcacatgtca gaacgcaacc ctgatcatgg ctgatcctgg ccttcgtgtg |
| 2041 | gttctgctaa ctatgtgcct gtcagtcttc cccaaaatct atgtggtcct caaatataac |
| 2101 | aactgtcatt caatacacat gtttgagcac ccagtgagct aagttttaag gattcaaaga |
| 2161 | tgaaaagtca tgctgtctcc tctgcaaagg gtgctcagac tagtgatgga aacagtatgg |
| 2221 | gatgaaagaa agcagaaggc cattgctgag caggcagtgg actcagcaga ggctgaaact |
| 2281 | atacaagtga cttggttcca gctgggccag caggataacc agatgaaaag aaggattgca |
| 2341 | tatattccat atatatttat gtttgaacaa agagtcaagg tttattgcaa ggataaggag |
| 2401 | gttttgttgg tggcctgtta agaccatcca gggtggtcat actggatagg gaagaaggtg |
| 2461 | agctggaaga ggaacagaca aacttggatg gccagatgtt gagatggagg agatggaggt |
| 2521 | cataacgtgg tcaaaaacat gttgatgaga ggacttagct acaaagttgt taacttaagc |
| 2581 | agaaacctca aggattgatt ttatgatttc tccaggaagt cctaaaagtt aatttcattt |
| 2641 | cagggagaaa aacaacagac cactgcaaag accaggaaca tgaaaggata atgtagtttg |
| 2701 | gtttgcttgg cagatacttg tgaaagatgt tggactgtaa ggctgtcaat atcctcctcg |
| 2761 | cagaacttac tacagtacat tgtatctgct cccttaccta cctgactctc ccactattca |
| 2821 | gtttgttcct taatggtaga ccatgcctga tcggtgtttt acacatcccc tgctatgtct |
| 2881 | gatacttgtg gatgctcaga aagtggggaa ggaaggaaag atacgatggt aaaaggctta |
| 2941 | cacatgtctt gagcagaatg ttcagtttggctcatttggct ggagtcatac tgcatggct |
| 3001 | gccattctgc tctggcatcc tcagagaagc acactgccca ttaaaggaaa aagggtgaat |
| 3061 | acaaatgttg agtcagaaca ctgcagacat ttagtaacct ccttcagagg aaaaaaaaag |
| 3121 | gtggggggaa tgacagaaat ccaaaaacta gtagagcttc cactttttca tttcagaaga |
| 3181 | aatcagttac tctcctctaa ggaccattac tattaacaaa acagagacct tagaaggaag |
| 3241 | cattatttac ttatcatata ttttgtaatg ttattaccct tcttgttata ctctttctta |
| 3301 | taccctacca ttgttagcag aaattatttt aaattaataa gatcctgcat gcttttcctt |
| 3361 | tttctaaaaa aagaaagatc tctgtgtaga atgtcctgtt ctgagccagt cctgagagga |
| 3421 | aaggaagtat aatcaatttg ttattaactg atgaaagaat taagtgaaag ataaacctta |
| 3481 | ggaagcagag ggaagttaat ctatgactaa gaaagttaag tactctgata actcattcat |
| 3541 | tccttctttt gttcatttac attatttaat cacaagtcta tgatgtgcca ggcactcagg |
| 3601 | aaatagtgaa aattggacac gcgatattct gcccttgtgt agcacacacc gtagtgggaa |
| 3661 | agaaagtgca cttttaaccg gacaactatc aacacgaaga ggggaggaag caggggctgg |
| 3721 | aaatgtccac agactttgcc aaagacaaag cccataatat ctgaaagtca gtttcttcca |
| 3781 | tcattttgtg tattaaggtt ctttattccc ctgttctctg ccttcctgct tgtcatcttc |
| 3841 | actcatcagc tgaccatgtt gcctcttacg gtgtaaactt gtaccagtct tatggtccct |
| 3901 | ctgggcagta cagccatgaa tttgatggag acgaggagtt ctatgtggac ctggagagga |
| 3961 | aggagactgt ctggcagttg cctctgttcc gcagatttag aagatttgac ccgcaatttg |
| 4021 | cactgacaaa catcgctgtg ctaaaacata acttgaacat cgtgattaaa cgctccaact |
| 4081 | ctaccgctgc taccaatggt atgtgtccac cattctgcct ttctttactg atttatccct |
| 4141 | ttataccaag tttcattatt ttctttccaa gaggtcccca gatcttctca tggcaattgc |
| 4201 | tgaaatttta tcatttctca tctctaaaat cacatattcc aatgtaatac aagggtcttt |
| 4261 | ccattatgca ttcattaaat ccttctagga gaggtctcat caaccttcta ctttattaaa |
| 4321 | catgcccaca gagagaaggg cacaggagta aagcagaggc aatgtgtcgt tgctcccaaa |
| 4381 | tgtgtcgtta caatgtgtcg ttgcttaccc aaagaggtaa ataaggcctc tttgaccagc |
| 4441 | aggagaggaa atgctggtag gaagactctt ccaggatgta atgcagaaga agctcagggc |
| 4501 | agagctattc acactttaca ccagtgctgt ttcctcacca tagaggttcc tgaggtcaca |
| 4561 | gtgttttcca agtctcccgt gacactgggt cagcccaaca ccctcatctg tcttgtggac |
| 4621 | aacatctttc ctcctgtggt caacatcacc tggctgagca atgggcactc agtcacagaa |
| 4681 | ggtgtttctg agaccagctt cctctccaag agtgatcatt ccttcttcaa gatcagttac |
| 4741 | ctcaccttcc tcccttctgc tgatgagatt tatgactgca aggtggagca ctggggcctg |
| 4801 | gatgagcctc ttctgaaaca ctggggtaag gatgagtttc accatttttt gatgctttct |
| 4861 | tgtctgtcaa gttcagaact tcctgccttt tactctatat cccaaaactt gttttccaca |
| 4921 | cttcatgagt ttcttttgtc ttttttttga aagaattaag caacaaaagc acagatttat |
| 4981 | taaaaaagaa agtacactcc acagggtggg agcaggcctg ccacttcatg ggtttctaat |
| 5041 | aacagacttc actctcctcc ctaagctggg ggccttgagt ctttgcagag ccaaccctct |
| 5101 | accccatccc atcccacaca catgcacatg agcaaactct gcattctgac ctcaacaact |
| 5161 | tcacttccac agagcctgag attccaacac ctatgtcaga gctcacagag actgtggtct |
| 5221 | gcgccctggg gttgtctgtg ggcctcgtgg gcattgtggt ggggaccgtc ttgatcatcc |
| 5281 | gaggcctgcg ttcagttggt gcttccagac accaagggcc cttgtgaatc ccatcctgaa |
| 5341 | aaggaaggta agattgagat ttgttagagc tgaagctgca ggaaggaaag t |

(SEQ ID NO: 11)
MILNKALMLGALALTTVMSPCGGEDIVADHVASYGVNLYQSYGP
SGQYSHEFDGDEEFYVDLERKETVWQLPLFRRFRRFDPQFALTNIAVLKHNLNIVIKR
SNSTAATNEVPEVTVFSKSPVTLGQPNTLICLVDNIFPPVVNITWLSNGHSVTEGVSE
TSFLSKSDHSFFKISYLTFLPSADEIYDCKVEHWGLDEPLLKHWEPEIPTPMSELTET
VVCALGLSVGLVGIVVGTVLIIRGLRSVGASRHQGPL

Human MHC class II HLA-DR-alpha mRNA GenBank: M60334.1 (SEQID NO:12)

| | |
|---|---|
| 1 | actcccaaaa gagcgcccaa gaagaaaatg gccataagtg gagtccctgt gctaggattt |
| 61 | ttcatcatag ctgtgctgat gagcgctcag gaatcatggg ctatcaaaga agaacatgtg |
| 121 | atcatccagg ccgagttcta tctgaatcct gaccaatcag gcgagtttat gtttgacttt |
| 181 | gatggtgatg agattttcca tgtggatatg gcaaagaagg agacggtctg gcggcttgaa |
| 241 | gaatttggac gatttgccag ctttgaggct caaggtgcat tggccaacat agctgtggac |
| 301 | aaagccaact tggaaatcat gacaaagcgc tccaactata ctccgatcac caatgtacct |
| 361 | ccagaggtaa ctgtgctcac gaacagccct gtggaactga gagagcccaa cgtcctcatc |
| 421 | tgtttcatag acaagttcac cccaccagtg gtcaatgtca cgtggcttcg aaatggaaaa |
| 481 | cctgtcacca caggagtgtc agagacagtc ttcctgccca gggaagacca ccttttccgc |
| 541 | aagttccact atctcccctt cctgccctca actgaggacg tttacgactg cagggtggag |
| 601 | cactggggct tggatgagcc tcttctcaag cactgggagt ttgatgctcc aagccctctc |
| 661 | ccagagacta cagagaacgt ggtgtgtgcc ctgggcctga ctgtgggtct ggtgggcatc |
| 721 | attattggga ccatcttcat catcaaggga ttgcgcaaaa gcaatgcagc agaacgcagg |
| 781 | gggcctctgt aaggcacatg gaggtgatgg tgtttctta |

(SEQ ID NO: 13)
MAISGVPVLGFFIIAVLMSAQESWAIKEEHVIIQAEFYLNPDQS
GEFMFDFDGDEIFHVDMAKKETVWRLEEFGRFASFEAQGALANIAVDKANLEIMTKRS
NYTPITNVPPEVTVLTNSPVELREPNVLICFIDKFTPPVVNVTWLRNGKPVTTGVSET
VFLPREDHLFRKFHYLPFLPSTEDVYDCRVEHWGLDEPLLKHWEFDAPSPLPETTENV
VCALGLTVGLVGIIIGTIFIIKGLRKSNAAERRGPL

Human MHC class II HLA-DR-beta (DR2-DQw1/DR4 DQw3) mRNA, GenBank: M20430. (SEQID NO:14)

| | |
|---|---|
| 1 | cttgcctgct tctctggccc ctggtcctgt cctgttctcc agcatggtgt gtctgaagct |
| 61 | ccctggaggc tcctgcatga cagcgctgac agtgacactg atggtgctga gctccccact |
| 121 | ggctttgtct ggggacaccc gaccacgttt cctgtggcag cctaagaggg agtgtcattt |
| 181 | cttcaatggg acggagcggg tgcggttcct ggacagatac ttctataacc aggaggagtc |
| 241 | cgtgcgcttc gacagcgacg tgggggagtt ccgggcggtg acggagctgg ggcggcctga |
| 301 | cgctgagtac tggaacagcc agaaggacat cctggagcag gcgcgggccg cggtggacac |
| 361 | ctactgcaga cacaactacg gggttgtgga gagcttcaca gtgcagcggc gagtccaacc |
| 421 | taaggtgact gtatatcctt caaagaccca gcccctgcag caccacaacc tcctggtctg |
| 481 | ctctgtgagt ggtttctatc caggcagcat tgaagtcagg tggttcctga acggccagga |
| 541 | agagaaggct gggatggtgt ccacaggcct gatccagaat ggagactgga ccttccagac |
| 601 | cctggtgatg ctggaaacag ttcctcgaag tggagaggtt tacacctgcc aagtggagca |
| 661 | cccaagcgtg acaagccctc tcacagtgga atggagagca cggtctgaat ctgcacagag |
| 721 | caagatgctg agtggagtcg ggggctttgt gctgggcctg ctcttccttg gggccgggct |
| 781 | gttcatctac ttcaggaatc agaaaggaca ctctggactt cagccaacag gattcctgag |
| 841 | ctgaaatgca gatgaccaca ttcaaggaag aactttctgc cccggctttg caggatgaaa |
| 901 | gctttcctgc ttggcagtta ttcttccaca agagagggct ttctcaggac ctggttgcta |
| 961 | ctggttcggc aactgcagaa aatgtcctcc cttgtggctt cctcagctcc tgcccttggc |
| 1021 | ctgaagtccc agcattgatg gcagcgcctc atcttcaact tttgtgctcc cctttgccta |
| 1081 | aaccgtatgg cctcccgtgc atctgtattc accctgtatg acaaacacat tacattatta |
| 1141 | aatgtttctc aaagatgg |

(SEQ ID NO: 15)
MVCLKLPGGSCMTALTVTLMVLSSPLALSGDTRPRFLWQPKREC
HFFNGTERVRFLDRYFYNQEESVRFDSDVGEFRAVTELGRPDAEYWNSQKDILEQARA
AVDTYCRHNYGVVESFTVQRRVQPKVTVYPSKTQPLQHHNLLVCSVSGFYPGSIEVRW
FLNGQEEKAGMVSTGLIQNGDWTFQTLVMLETVPRSGEVYTCQVEHPSVTSPLTVEWR
ARSESAQSKMLSGVGGFVLGLLFLGAGLFIYFRNQKGHSGLQPTGFLS

The SAg-MHCII transduced T cells are self-activating as the SAg-MHCII complex inducibly expressed on the T cell surface engages the vβ region of the TCR constitutively expressed on the surface of the same T cell. In this process the SAg binds to the constitutively expressed costimulatory CD28 binding domain which is also constitutively expressed on the T cell surface. Autoactivation of T cell proliferation occurs after engagement of the T cell's cognate tumor specific receptor ligand with its cognate receptor on tumor cells. When such T cells are introduced in vivo, they interface with their cognate receptor or antigen expressed on the tumor cell. This interface initiates T cell transcription/expression of SAg and MHCII with consequent activation of adjacent TCR vβ leading to T cell proliferation and release granzymes/perforin from cytotoxic T cells that lyse the tumor cells.

When the SAg/MHCII transduced T cells contact their cognate tumor specific target, SAg and MHCII are simultaneously synthesized. The SAg/MHCII complex thereupon binds its cognate TCR vβ receptor and its B7 domains simultaneously binds to CD28 constitutively expressed on the T cell surface. The latter triggers T cell proliferation and granzyme/perforin production leading to tumor cell killing. Such B7 binding to constitutive CD28 prevent T cell AICD ensuring the survival of the T cells in vivo. When the T cells with surface bound tumor specific fv or tumor receptor ligand are introduced into the tumor bearing host, they bind to and kill the tumor cell.

In the engineered T cell constitutively expressed CD28 is activated by the B7 binding domain present in the SAg. The latter along with MHCII is inducibly expressed after binding of tumor receptor ligand to the tumor target cell. Such expression is conferred by zeta signaling domain which is linked in frame in the retroviral vector to nucleic acids encoding the tumor receptor ligand, the SAg and MHCII molecules. In this construct, a tumor specific fv can be used in place of the tumor receptor ligand. Expression of T cell SAg and MHCII therefore occurs only after the transduced T cell tumor specific receptor/fv antibody binds to its cognate receptor on the tumor cell. This property ensures that the T cell can migrate to the tumor cells without diversion to MHCII binding molecules and cells in the host. Moreover, SAg activation of T cell costimulatory receptor CD28 enables persistence and survival of adoptively transferred engineered T cells in the human circulation and solid tumors without undergoing senescence or AICD.

The targeting molecule used to redirect T cells to the tumor specific receptor ligand such as the EGF ligand or a tumor specific single-chain fv variable fragment from a monoclonal antibody containing a transmembrane hinge region, and a signaling domain (typically the zeta chain from the T-cell signaling complex). The synthetic human EGF DNA fragment (Takara, Dalian, China) encoding 53 amino acids is useful in this invention but any other tumor receptor ligand may be employed for purposes of targeting the T lymphocyte to the tumor or tumor vasculature or tumor stroma. The EGF 53-amino acid polypeptide of known sequence contains six cysteine residues, which are thought to form three intrachain disulphide bonds. Examination of the precursor 4,706-bp nucleotide sequence reveals one translational reading frame that codes for an uninterrupted sequence of 1,168 amino acid residues. The 53 amino acids of mouse EGF were found to be embedded within this sequence. The open reading frame that includes the EGF amino acid sequence starts with an initiation codon ATG, three nucleotides downstream from an in-frame termination codon (TAA). The initiator methionine is followed by a stretch of 29 amino acids which are hydrophobic. The sequence of the mature EGF protein begins at position 977 of the precursor and ends at amino acid 1,029 as shown in FIG. 2A of Gray et al., Nature 303: 722-725 (1983). The tumor specific fv comprises nucleic acids encoding the single-chain Fv from the hypervariable region of a human tumor specific antibody and a hinge region, the tumor specific fv is operably linked in frame to the SAg and the extracellular domain of an MHCII molecule and the intracellular domains of the CD3 intracellular signaling domain. The lentiviral vector with three coding regions is used to incorporate the nucleic acids encoding the latter 4 constructs and is used for gene transfer into resting T cells or cytotoxic T cells. The tumor specific fv fragment, superantigen, truncated form of the full length TCR-c intracellular signaling domain in cis are integrated into the same lentiviral vector.

The complete sequences are amplified directly from the plasmids by PCR. Constructs containing the SAg and extracellular domains or alpha chain or beta chain of the MHCII in combination with the intracellular domain of the CD3 zeta chain (ζ) and the extracellular growth factor (EGF) are generated by the procedure of splicing by overlap extension. A plasmid encoding a EGF-SAg-MHCII-CD3 zeta chain chimeric protein and the above constructs are used as templates for PCR. The resulting PCR fragments containing the complete construct are then cloned into peps 3′ of the promoter using standard molecular biology techniques. pELPS is a derivative of the third-generation lentiviral vector pRRL-SIN-CMV-eGFPWPRE in which the CMV promoter is replaced with the EF-1α promoter and the central polypurine tract of HIV is inserted 5′ of the promoter. All constructs are verified by sequencing. Lentiviral gene transfer permits generation of large numbers of EGF-SAg-MHCII-CD3zeta T cells after contact with the tumor cell EGF receptor. Lentiviral transduction of T cells at a multiplicity of infection (MOI) of MOI of ˜8 on day 1 with vector expressing enhanced GFP under the control of the promoter results in surface expression of the EGF or TSfv specific antibody in T cells and cytotoxic T cells.

Safety measures include the infusion of lower numbers of T cells, the use of immunosuppressive agents, and the introduction of an inducible “suicide signal” to kill the cells when they are creating mischief; a novel, non-immunogenic, inducible caspase 9 suicide gene has been developed for this purpose.

Operationally, four days after receiving chemotherapy with pentostatin and cyclophosphamide for depletion of lymphocytes, the patient receive 1.42×10⁷ transduced T cells over 3 days with no additional cytokines. The transduced T cells show an increase in the number of circulating TS antigen receptor-positive T cells to a level nearly 1000 times as high as the level detected the day after infusion. These cell produce a low level of cytokines due to the low cytokine (interferon-γ and interleukin-6). Eight months after therapy, tumor specific antigen/receptor-positive T cells persist, and patients have no evidence of disease on physical examination or on computed tomographic, flow-cytometric, or cytogenetic analysis. The expansion, persistence, and development of the memory phenotype in addition to the antitumor effects of these T cells is significant.

The term “T cells” as used herein refers to lymphocytes that mature in the thymus and are chiefly responsible for cell-mediated immunity. T cells are involved in the adaptive immune system. The term “natural killer (NK) cells” as used herein refers to lymphocytes that are part of cell-mediated immunity and act during the innate immune response. They do not require prior activation in order to perform their cytotoxic effect on target cells. Cytotoxic T cells (CTL or killer T cells) are a subset of T lymphocytes capable of inducing the death of infected somatic or tumor cells.

For initial genetic modification of the T cells to provide tumor receptor or tumor antigen-specificity, a retroviral vector is generally employed for transduction, however any other suitable viral vector or delivery system can be used. For subsequent genetic modification of the cells to provide cells comprising a tumor antigen binding receptor along with MHCII molecules retroviral gene transfer (transduction) likewise proves effective. Combinations of retroviruses and an appropriate packaging line are also suitable, where the capsid proteins are functional for infecting human cells. Various amphotropic virus-producing cell lines are known, including, but not limited to PA12 (Miller, et al. Mol. Cell. Biol. 5:431-437 (1985); PA317 (Miller, et al. Mol. Cell. Biol. 6:2895-2902 (1986)); and CRIP (Danos, et al. Proc. Natl. Acad. Sci. USA 85:6460-6464 (1988)). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. Blood 80:1418-1422 (1992) or culturing with viral supernatant alone or concentrated vector stocks with or without appropriate growth factors and polycations, e.g., by the method of Xu, et al. Exp. Hemat. 22:223-230 (1994); and Hughes, et al., J. Clin. Invest. 89:1817 (1992). Other transducing viral vectors are used to express a co-stimulatory ligand of the invention in a T cell or NKT cell. Preferably, the chosen vector exhibits high efficiency of infection and stable integration and expression (see, e.g., Cayouette et al., Human Gene Therapy 8:423-430 (1997); Kido et al., Current Eye Research 15:833-844 (1996); Bloomer et al., Journal of Virology 71:6641-6649 (1997); Naldini et al., Science 272:263-267 (1996); and Miyoshi et al., Proc. Natl. Acad. Sci. U.S.A. 94:10319 (1997). Other useful viral vectors include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14 (1990); Friedman, Science 244: 1275-1281 (1989); Eglitis et al., BioTechniques 6:608-614 (1988); Tolstoshev et al., Current Opinion in Biotechnology 1:55-61 (1990); Sharp, Lancet 337:1277-1278 (1991); Cornetta et al., Nucleic Acid Research and Molecular Biology 36:311-322 (1987); Anderson, Science 226:401-409 (1984); Moen, Blood Cells 17:407-416 (1991); Miller et al., Biotechnology 7:980-990 (1989); Le Gal La Salle et al., Science 259:988-990 (1993); Johnson, Chest 107:77S-83S (1995). Retroviral vectors are particularly well developed and have been used in clinical settings (Rosenberg et al., N. Engl. J. Med 323:370 (1990); Anderson et al., U.S. Pat. No. 5,399,346).

Non-viral approaches can also be employed for the expression of a protein in cell. For example, a nucleic acid molecule can be introduced into a cell by administering the nucleic acid in the presence of lipofection (Feigner et al., Proc. Natl. Acad. Sci. U.S.A. 84:7413 (1987); Ono et al., Neuroscience Letters 17:259 (1990); Brigham et al., Am. J. Med. Sci. 298:278, (1989; Staubinger et al., Methods in Enzymology 101:512, (1983), asialoorosomucoid-polylysine conjugation (Wu et al., J Biol Chem 263: 14621 (1988); Wu et al., J Biol Chem 264:16985 (1989), or by micro-injection under surgical conditions (Wolff et al., Science 247:1465 (1990)). Other non-viral means for gene transfer include transfection in vitro using calcium phosphate, DEAE dextran, electroporation, and protoplast fusion. Liposomes are also beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above. The resulting cells can then be grown under conditions similar to those for unmodified cells, whereby the modified cells can be expanded and used for a variety of purposes.

### Cells for Use in the Methods of the Invention

The present invention provides T cells or any T cell subset expressing at least one tumor antigen-recognizing receptor and a whole MHCII molecule or an MHCII alpha or beta chain, a co-stimulatory ligand such as CD28 which can interact with a superantigen and a superantigen or superantigen homologue or fusion protein and methods of using such cells for the treatment of a cancer. MHC class II positive CD8+ T cells are particularly preferred. In one approach, tumor antigen-specific T cells, CTL cells are used as shuttles for the selective enrichment of one or more co-stimulatory ligands for the treatment or prevention of neoplasia. For example, a T cell expressing a SEG and MHCII alpha chain are constitutively co-expressed in a T cell that expresses a tumor receptor such as EGF that recognizes and binds the EGF receptor on lung, breast and colon carcinoma cells or tumor antigen receptor such as PZ1 that similarly recognizes and binds Prostate Specific Membrane Antigen (PSMA). Such cells are administered to a human subject in need thereof for the treatment or prevention of lung, breast, colon or prostate cancer.

Types of tumor antigen or receptor-specific human lymphocytes that can be used in the methods of the invention include, without limitation, peripheral donor lymphocytes genetically modified to express tumor specific antigen receptors (CARs) (Sadelain, M., et al. Nat Rev Cancer 3:35-45 (2003), peripheral donor lymphocytes genetically modified to express a full-length tumor antigen-recognizing T cell receptor complex comprising the α and β heterodimer (Morgan, R. A., et al. 2006 Science 314:126-129), lymphocyte cultures derived from tumor infiltrating lymphocytes (TILs) in tumor biopsies (Panelli, M. C, et al. J Immunol 164:495-504 (2000); Panelli, M. C, et al. J Immunol 164:4382-4392 (2000), and selectively in vitro-expanded antigen-specific peripheral blood leukocytes employing artificial antigen-presenting cells (AAPCs) or pulsed dendritic cells (Dupont, J., et al. Cancer Res 65:5417-5427 (2005); Papanicolaou, G. A., et al. Blood 102: 2498-2505 (2003). The T cells may be autologous, allogeneic, xenogeneic or derived in vitro from engineered progenitor or stem cells.

Any suitable or tumor receptor ligand or tumor specific antibody or fv/Fab is suitable for use in binding to the tumor-related embodiments described herein. Sources of tumor specific peptide include, but are not limited to cancer proteins. The antigen can be expressed as a peptide or as an intact protein or portion thereof. The intact protein or a portion thereof can be native or mutagenized. One suitable antigen is prostate specific membrane antigen (PSMA) and one suitable tumor receptor is the EGF receptor.

The unpurified source of CTLs may be any known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-CTLs initially. Monoclonal antibodies are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. Preferably, at least about 80%, usually at least 70% of the total hematopoietic cells will be removed prior to cell isolation. Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a monoclonal antibody, including, but not limited to, complement and cytotoxins; and panning with antibody attached to a solid matrix, e.g. plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells are selected against dead cells, by employing dyes associated with dead cells such as propidium iodide (PI). Preferably, the cells are collected in a medium comprising 2% fetal calf serum (FCS) or 0.2% bovine serum albumin (BSA) or any other suitable, preferably sterile, isotonic medium.

Accordingly, the invention generally provides a cytotoxic T cell, such as a tumor specific T cell comprising a receptor ligand that binds to a tumor receptor, an exogenous superantigen, an extracellular MHCII ligand for SAg (e.g. Alpha or beta chain, or DR, DQ, DP subset) and a CD3 zeta chain. The expression of the SAg and MHCII SAg ligand occur only on activation of the CD3 zeta chain upon binding of the Tcell receptor ligand to the tumor receptor.

### Vectors

Genetic modification of immunoresponsive cells (e.g., T cells, CTL cells, NK cells) can be accomplished by transducing a substantially homogeneous cell composition with a recombinant DNA construct. Preferably, a retroviral vector (either gamma-retroviral or lentiviral) is employed for the introduction of the DNA construct into the cell. For example, a polynucleotide encoding a SAg, an MHCII molecule, or a receptor that binds an antigen, or a variant, or a fragment thereof, is cloned into a retroviral vector and expression can be driven from its endogenous promoter, from the retroviral long terminal repeat, or from a promoter specific for a target cell type of interest. Non-viral vectors may be used as well.

### Superantigens

### Production and Isolation of Superantigens

The superantigens disclosed herein are prepared by either biochemical isolation, or, preferably by recombinant methods. The following SAgs, including their sequences and biological activities have been known for a number of years. Studies of these SAgs are found throughout the biomedical literature. For, biochemical and recombinant preparation of these SAgs see the following references: Borst D W et al., Infect. Immun. 61: 5421-5425 (1993); Couch J L et al., J. Bacteriol. 170: 2954-2960 (1988); Jones C L et al., J. Bacteriol. 166: 29-33 (1986); Bayles K W et al., J. Bacteriol. 171: 4799-4806 (1989); Blomster-Hautamaa, D A et al., J. Biol. Chem. 261:15783-15786 (1986); Johnson, L P et al., Mol. Gen. Genet. 203, 354-356 (1986); Bohach G A et al., Infect. Immun. 55: 428-433 (1987); Iandolo J J et al., Meth. Enzymol 165:43-52 (1988); Spero L et al., Meth. Enzymol 78 (Pt A):331-6 (1981); Blomster-Hautamaa D A, Meth. Enzymol 165: 37-43 (1988); Iandolo J J Ann. Rev. Microbiol. 43: 375-402 (1989); U.S. Pat. No. 6,126,945 and U.S. provisional patent application 60/389,366 filed Jun. 15, 2002. These references and the references cited therein are hereby incorporated by reference in their entirety.

These SAgs are Staphylococcal enterotoxin A (SEA), Staphylococcal enterotoxin B (SEB), Staphylococcal enterotoxin C (SEC—actually three different proteins, SEC1, SEC2 and SEC3)), Staphylococcal enterotoxin D (SED), Staphylococcal enterotoxin E (SEE) and toxic shock syndrome toxin-1 (TSST-1) (U.S. Pat. No. 6,126,945 and U.S. provisional patent application 60/389,366 filed Jun. 15, 2002, and the references cited therein). The amino acids sequences of the above group of native (wild-type) SAgs are given in the following: SEA (Huang I Y et al., J. Biol. Chem. 262:7006-7013 (1987));

SEB (Papageorgiou A C et al. J. Mol. Biol. 277:61-79 (1998); SEC1 (Bohach G A et al., Mol. Gen. Genet. 209:15-20 (1987); SEC2 (Papageorgiou A C et al., Structure 3:769-779 (1995); SEC3 (Hovde C J et al., Mol. Gen. Genet. 220:329-333 (1990); SED (Bayles K W et al., J. Bacteriol. 171:4799-4806 (1989); SEE (Couch J L et al., J. Bacteriol. 170:2954-2960 (1988); TSST-1 (Prasad G S et al., Protein Sci. 6:1220-1227 (1997)).

The sections which follow discuss SAgs which have been discovered and characterized more recently.

Staphylococcal Enterotoxins SEG, SEH, SEI, SEJ, SEK, SEL, SEM, SEN, SEO, SEP, SEQ, SER, SEU

New Staphylococcal enterotoxins G, H, I, J, K, L and M (SEG, SEH, SEI, SEJ, SEK, SEL, SEM, SEN, SEO, SEP, SEQ, SER, SEU; abbreviated below as “SEG-SEU”) were described in Jarraud, S. et al., J. Immunol. 166: 669-677 (2001); Jarraud S et al., J. Clin. Microbiol. 37: 2446-2449 (1999) and Munson, S H et al., Infect. Immun. 66:3337-3345 (1998). SEG-SEU show superantigenic activity and are capable of inducing tumoricidal effects. The homology of these SE's to the better known SE's in the family ranges from 27-64%. Each induces selective expansion of TCR Vβ subsets. Thus, these SEs retain the characteristics of T cell activation and Vβ usage common to all the other SE's. RT-PCR was used to show that SEH stimulates human T cells via the Vα domain of TCR, in particular Vα (TRAV27), while no TCR Vβ-specific expansion was seen. This is in sharp contrast to all other studied bacterial superantigens, which are highly specific for TCR Vβ. Vβ binding superantigens form one group, whereas SEH has different properties that fit well with Vα reactivity. It is suggested that SEH directly interacts with the TCR Vα domain (Petersson K et al., J Immunol. 170:4148-54 (2003)).

SEG and SEH of this group and other enterotoxins including SPEA, SPEC, SPEG, SPEH, SME-Z, SME-Z2, (see below) utilize zinc as part of high affinity MHC class II receptor. Amino acid substitution(s) at the high-affinity, zinc-dependent class II binding site are created to reduce their affinity for MHC class II molecules.

Jarraud S et al., 2001, supra, discloses methods used to identify and characterize egc SEs SEG-SEM, and for cloning and recombinant expression of these proteins. The egc comprises SEG, SEI, SEM, SEN, SEO and pseudogene products designated ψent 1 and ψent 2. Purified recombinant SEN, SEM, SEI, SEO, and SEGL29P (a mutant of SEN) were expressed in E. coli. Recombinant SEG, SEN, SEM, SEI, and SEO consistently induced selective expansion of distinct subpopulations of T cells expressing particular Vβ genes.

Jarraud S et al., 2001, supra, indicates that the seven genes and pseudogenes composing the egc (enterotoxin gene cluster) operon are co-transcribed. The association of related co-transcribed genes suggested that the resulting peptides might have complementary effects on the host's immune response. One hypothesis is that gene recombination created new SE variants differing by their superantigen activity profiles. By contrast, SEGL29P failed to trigger expansion of any of 23 Vβ subsets, and the L29P mutation accounted for the complete loss of superantigen activity (although this mutation did not induce a major conformational change). It is believed that this substitution mutation located at a position crucial for proper superantigen/MHC II interaction.

Overall, TCR repertoire analysis confirms the superantigenic nature of SEG, SEI, SEM, SEN, SEO. These investigators used a number of TCR-specific mAbs (Vβ specificity indicated in brackets) for flow cytometric analysis: E2.2E7.2 (Vβ2), LE89 (Vβ3), IMMU157 (Vβ5.1), 3D11 (Vβ5.3), CRI304.3 (Vβ6.2), 3G5D15 (Vβ7), 56C5.2 (Vβ8.1/8.2), FIN9 (Vβ9), C21 (Vβ11), S511 (Vβ12), IMMU1222 (Vβ13.1), JIJ74 (Vβ13.6), CAS1.1.13 (Vβ14), Tamaya1.2 (Vβ16), E17.5F3 (Vβ17), βA62.6 (Vβ18), ELL1.4 (Vβ20), IG125 (Vβ21.3), IMMU546 (Vβ22), and HUT78.1 (Vβ23). Flow cytometry also revealed preferential expansion of CD4+ T cells in SEI and SEM cultures. By contrast, the CD4/CD8 ratios in SEO-, SEN-, and SEG-stimulated T cell lines were close to those in fresh PBL.

Recombinant and biochemical preparation of the egc SEs is given in U.S. 60/799,514, PCTUS05/022,638, U.S. 60/583,692, U.S. 60/665,654, U.S. 60/626,159 which incorporated by reference and their references in their entirety.

The amino acid sequences of SEG-SEU are as follows: SEG (Baba, T. et al., Lancet 359, 1819-1827 (2002)); SEG (Jarraud, S et al., J. Immunol. 166: 669-677 (2001)); SEH (Omoe, K. et al., J. Clin. Microbiol. 40: 857-862 (2002)); SEI (Kuroda, M. et al., Lancet 357 (9264), 1225-1240 (2001)); SEJ (Zhang S. et al., FEMS Microbiol. Lett. 168:227-233 (1998)); SEK (Baba T., et al., Lancet 359: 1819-1827 (2002)); SEL (Kuroda M. et al., Lancet 357: 1225-1240 (2001)); SEM (Kuroda M. et al., Lancet 357: 1225-1240 (2001)); SEN (Jarraud S et al., J. Immunol. 166: 669-677 (2001)); SEO (Jarraud S et al., J. Immunol. 166: 669-677 (2001)); ψent 1 (Jarraud S et al., J. Immunol. 166: 669-677 (2001)); ψent 2 (Jarraud S et al., J. Immunol. 166: 669-677 (2001)); SEP (Kuroda M. et al., Lancet 357, 1225-1240 (2001); SEQ (Lindsay, J A et al., Mol. Microbiol. 29, 527-543 (1998)); SER Omoe K et al., ACCESSION BAC97795; SEU (Letertre C et al., J. Appl. Microbiol. 95, 38-43 (2003))

### Streptococcal Pyrogenic Exotoxins (SpEs)

The SpE's SPEA, SPEB, SPEC, SPEG, SPEH, SME-Z, SME-Z2 and SSA are superantigens induce tumoricidal effects. SPEA, SPEB, SPEC have been known for some time and their structures and biological activities described in numerous publications. SPEG, SPEH, and SPEJ genes were identified from the Streptococcus pyogenes M1 genomic database and described in detail in Proft, T et al., J. Exp. Med. 189: 89-101 (1999) which also describes SMEZ, SMEZ-2. This document also describes the cloning and expression of the genes encoding these proteins.

The smez-2 gene was isolated from the S. pyogenes strain 2035, based on sequence homology to the streptococcal mitogenic exotoxin z (smez) gene. SMEZ-2, SPE-G, and SPE-J are most closely related to SMEZ and SPEC, whereas SPEH is most similar to the SEs than to any other streptococcal toxin. As described by Proft, T et al supra, rSMEZ, rSMEZ-2, rSPE-G, and rSPE-H were mitogenic for human peripheral blood T lymphocytes. SMEZ-2 appears to be the most potent SAg discovered thus far.

All these toxins, except rSPE-G, were active on murine T cells, but with reduced potency. Binding to a human B-lymphoblastoid line was shown to be zinc dependent with high binding affinity of 15-65 nM. Analysis of competition for binding between toxins of this group revealed overlapping but discrete binding to subsets of class II molecules in the hierarchical order (SMEZ, SPE-C)>SMEZ-2>SPE-H>SPE-G. The most common targets for these SAgs were human Vβ2.1- and Vβ4-expressing T cells.

### Streptococcus Pyrogenic Exotoxin A (SPEA)

SPEA can be purified from cultures of S. pyogenes as described by Kline et al., Infect. Immun. 64:861-869 (1996). Plasmids that include the spea1 gene which encode SPEA, and the expression and purification of recombinant SPEA (“rSPEA”) are described by Kline et al., supra. The native SPEA sequence is given in Papageorgiou, A. C. et al., EMBO J. 18:9-21 (1999).

### Streptococcus Pyrogenic Exotoxin B (SPEB)

Purification of native SPEB is described by Gubba, S. et al., Infect. Immun. 66: 765-770 (1998). Expression and purification of recombinant SPEB are also described in this reference. The native SPEB sequence is given in Kapur, V. et al., Microb. Pathog. 15:327-346 (1993). Streptococcus Pyrogenic Exotoxin C (SPEC)

Methods of isolation and characterization of SPEC is carried out by the methods of Li, P L et al., J. Exp. Med. 186: 375-383 (1997). These references also describe T cell proliferation stimulated by this SAg and the analysis of its selectivity for TCR Vβ regions. The native sequence of SPEC is given in Kapur, V. et al., Infect. Immun. 60: 3513-3517 (1992).

Streptococcal superantigen (SSA)

SSA is a ˜28-kDa superantigen protein isolated from culture supernatants as described by Mollick J et al., J. Clin. Invest. 92: 710-719 (1993) and Reda K et al., Infect. Immun. 62: 1867-1874 (1994). SSA stimulates proliferation of human T cells bearing Vβ1, Vβ3, Vβ5.2, and Vβ15 in an MHC class II-dependent manner. The first 24 amino acid residues of SSA are 62.5% identical to SEB, SEC1, and SEC3. Purification and cloning of SSA is described in Reda K et al., Infect. Immun. 62: 1867-1874 (1994). The native sequence of SSA is given in Reda, K B. et al., Infect. Immun. 64: 1161-1165 (1996).

### Streptococcal Pyrogenic Exotoxins G and H and SMEZ

The sequences of the more recently discovered Streptococcal exotoxin SAgs are provided below:

Yersinia pseudotuberculosis Mitogen (Superantigen) (YPM)

Cloning, expression and purification of YPM is described by Miyoshi-Akiyama, T. et al., J. Immunol. 154: 5228-5234 (1995). The above reference described assays of YPM using lymphoid cells and murine L cells transfected with human HLA genes, including T cell proliferation and cytokine (IL2) secretion.

### Staphylococcal Exotoxin Like Proteins (SET)

The identification characterization of the SETs (SET-1 and SET-2) and the cloning and purification of SET-1 is described in Williams, R. J. et al., Infect. Immun. 68: 4407-4414 (2000). This reference discloses the distribution of the set1 gene among Staphylococcal species and strains. The set1 nucleotide sequences are deposited in the GenBank database under accession numbers AF094826 (set gene cluster fragment), AF188835 (NCTC 6571 set1 gene), AF188836 (FRI326 set1gene), and AF188837 (NCTC 8325-4 set1 gene). Recombinant SET-1 protein stimulates production of the proinflammatory cytokines IL-1β, IL-6, and TNFα

### Functional Homologues and Derivatives of Tumoricidal Proteins, Superantigens or Peptides

The present invention contemplates, in addition to native proteins incorporated in the sickle cells, the use of homologues of native proteins that have the requisite biological activity to be useful in accordance with the invention.

Thus, in addition to native proteins and nucleic acid compositions described herein, the present invention encompasses functional derivatives, among which homologues are preferred. By “functional derivative” is meant a “fragment,” “variant,” “mutant,” “homologue,” “analogue,” or “chemical derivative. Homologues include fusion proteins, chimeric proteins and conjugates that include a SAg portion fused to or conjugated to a fusion partner polypeptide or peptide. A functional derivative retains at least a portion of the biological activity of the native protein which permits its utility in accordance with the present invention. For superantigens, such biological activity includes stimulation of T cell proliferation and/or cytokine secretion, stimulation of T cell-mediated cytotoxic activity, as a result of interactions of the SAg composition with T cells preferably via the TCR Vβ or Vα region. For pseudomonas exotoxin A, a homologue must retain tumor cell cytolytic activity.

A “fragment” refers to any shorter peptide. A “variant” refers to a molecule substantially similar to either the entire protein or a peptide fragment thereof. Variant peptides may be conveniently prepared by direct chemical synthesis of the variant peptide, using methods well-known in the art.

A homologue refers to a natural protein, encoded by a DNA molecule from the same or a different species. Homologues, as used herein, typically share at least about 50% sequence similarity at the DNA level or at least about 18% sequence similarity at the amino acid level, with a native protein.

An “analogue” refers to a non-natural molecule substantially similar to either the entire molecule or a fragment thereof

A “chemical derivative” contains additional chemical moieties not normally a part of the peptide. Covalent modifications of the peptide are included within the scope of this invention.

Such modifications may be introduced into the molecule by reacting targeted amino acid residues of the peptide with an organic derivatizing agent that is capable of reacting with selected side chains or terminal residues.

A fusion protein comprises a native protein, a fragment or a homologue fused by recombinant means to another polypeptide fusion partner, optionally including a spacer between the two sequences. Preferred fusion partners are antibodies, Fab fragments, single chain Fv fragments. Other fusion partners are any peptidic receptor, ligand, cytokine, domain (“ECD”) of a molecule and the like.

The recognition that the biologically active regions of the proteins, for example, are substantially homologous, i.e., that the sequences are substantially similar, enables prediction of the sequences of synthetic peptides which will exhibit similar biological effects in accordance with this invention.

The following terms are used in the disclosure of sequences and sequence relationships between two or more nucleic acids or polypeptides: (a) “reference sequence”, (b) “comparison window”, (c) “sequence identity”, (d) “percentage of sequence identity”, and (e) “substantial identity” As used herein, “reference sequence” is a defined sequence used as a basis for sequence comparison. A reference sequence may be a subset or the entirety of a specified sequence; for example, as a segment of a full-length cDNA or other polynucleotide sequence, or the complete cDNA or polynucleotide sequence. The same is the case for polypeptides and their amino acid sequences.

As used herein, “comparison window” includes reference to a contiguous and specified segment of a polynucleotide or amino acid sequence, wherein the sequence may be compared to a reference sequence and wherein the portion of the sequence in the comparison window may comprise additions or deletions (i.e., gaps) compared to the reference sequence (which does not comprise additions or deletions) for optimal alignment of the two sequences. Generally, the comparison window is at least 20 contiguous nucleotides or amino acids in length, and optionally can be 30, 40, 50, 100, or longer. Those of skill in the art understand that to avoid a high similarity to a reference sequence due to inclusion of gaps in the sequence a gap penalty is typically introduced and is subtracted from the number of matches.

Methods of alignment of nucleotide and amino acid sequences for comparison are well-known in the art. For comparison, optimal alignment of sequences may be done using any suitable algorithm, of which the following are examples:

(a) the local homology algorithm (“Best Fit”) of Smith and Waterman, Adv. Appl. Math. 2: 482 (1981);
(b) the homology alignment algorithm (GAP) of Needleman and Wunsch, J. Mol. Biol. 48: 443 (1970); or
(c) a search for similarity method (FASTA and TFASTA) of Pearson and Lipman, Proc. Natl. Acad. Sci. 85 2444 (1988);

In a preferred method of alignment, Cys residues are aligned. Computerized implementations of these algorithms, include, but are not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG) (Madison, Wis.). The CLUSTAL program is described by Higgins et al., Gene 73:237-244 (1988); Higgins et al., CABIOS 5:151-153 (1989); Corpet et al., Nuc Acids Res 16:881-90 (1988); Huang et al., CABIOS 8:155-65 (1992), and Pearson et al., Methods in Molecular Biology 24:307-331 (1994).

A preferred program for optimal global alignment of multiple sequences is PileUp (Feng and Doolittle, J Mol Evol 25:351-360 (1987) which is similar to the method described by Higgins et al., 1989, supra). The BLAST family of programs which can be used for database similarity searches includes: NBLAST for nucleotide query sequences against database nucleotide sequences; XBLAST for nucleotide query sequences against database protein sequences; BLASTP for protein query sequences against database protein sequences; TBLASTN for protein query sequences against database nucleotide sequences; and TBLASTX for nucleotide query sequences against database nucleotide sequences. See, for example, Ausubel et al., eds., Current Protocols in Molecular Biology, Chapter 19, Greene Publishing and Wiley-Interscience, New York (1995) or most recent edition. Unless otherwise stated, stated sequence identity/similarity values provided herein, typically in percentages, are derived using the BLAST 2.0 suite of programs (or updates thereof) using default parameters. Altschul et al., Nuc Acids Res. 25:3389-3402 (1997).

As is known in the art, BLAST searches assume that proteins can be modeled as random sequences. However, many real proteins comprise regions of nonrandom sequence which may include homopolymeric tracts, short-period repeats, or regions rich in particular amino acids. Alignment of such regions of “low-complexity” regions between unrelated proteins may be performed even though other regions are entirely dissimilar. A number of low-complexity filter programs are known that reduce such low-complexity alignments. For example, the SEG (Wooten et al., Comput. Chem. 17:149-163 (1993) and XNU (Claverie et al., Comput. Chem, 17:191-201 (1993) low-complexity filters can be employed alone or in combination.

As used herein, “sequence identity” or “identity” in the context of two nucleic acid or amino acid sequences refers to the residues in the two sequences which are the same when aligned for maximum correspondence over a specified comparison window. It is recognized that when using percentages of sequence identity for proteins, a residue position which is not identical often differs by a conservative amino acid substitution, where a substituting residue has similar chemical properties (e.g., charge, hydrophobicity, etc.) and therefore does not change the functional properties of the polypeptide. Where sequences differ in conservative substitutions, the % sequence identity may be adjusted upwards to correct for the conservative nature of the substitution, and be expressed as “sequence similarity” or “similarity” (combination of identity and differences that are conservative substitutions). Means for making this adjustment are well-known in the art. Typically this involves scoring a conservative substitution as a partial rather than as a full mismatch, thereby increasing the percentage sequence identity. Thus, for example, where an identical amino acid is given a score of “1” and a non-conservative substitution is given a score of “0” zero, a conservative substitution is given a score between 0 and 1. The scoring of conservative substitutions is calculated, e.g., according to the algorithm of Meyers et al., CABIOS 4:11-17 (1988) as implemented in the program PC/GENE (Intelligenetics, Mountain View, Calif., USA).

As used herein, “percentage of sequence identity” refers to a value determined by comparing two optimally aligned sequences over a comparison window, wherein the portion of the nucleotide or amino acid sequence in the comparison window may comprise additions or deletions (i.e., gaps) as compared to the reference sequence (which lacks such additions or deletions) for optimal alignment, such as by the GAP algorithm (supra). The percentage is calculated by determining the number of positions at which the identical nucleotide or amino acid residue occurs in both sequences to yield the number of matched positions, dividing that number by the total number of positions in the window of comparison and multiplying the result by 100, thereby calculating the percentage of sequence identity.

The term “substantial identity” of two sequences means that a polynucleotide or polypeptide comprises a sequence that has at least 60%, preferably at least 70%, more preferably at least 80%, even more preferably at least 90%, and most preferably at least 95% sequence identity to a reference sequence using one of the alignment programs described herein using standard parameters. Values can be appropriately adjusted to determine corresponding identity of the proteins encoded by two nucleotide sequences by taking into account codon degeneracy, amino acid similarity, reading frame positioning, etc.

One indication that two nucleotide sequences are substantially identical is if they hybridize to one other under stringent conditions. Because of the degeneracy of the genetic code, a number of different nucleotide codons may encode the same amino acid. Hence, two given DNA sequences could encode the same polypeptide but not hybridize under stringent conditions. Another indication that two nucleic acid sequences are substantially identical is that the polypeptide encoded by the first nucleic acid is immunologically cross reactive with the polypeptide encoded by the second nucleic acid. Clearly, then, two peptide or polypeptide sequences are substantially identical if one is immunologically reactive with antibodies raised against the other. A first peptide is substantially identical to a second peptide, if they differ only by a conservative substitution. Peptides which are “substantially similar” share sequences as noted above except that nonidentical residue positions may differ by conservative substitutions.

Thus, in one embodiment of the present invention, the Lipman-Pearson FASTA or FASTP program packages (Pearson, W. R. et. al., 1988, supra; Lipman, D. J. et al, Science 227:1435-1441 (1985)) in any of its older or newer iterations may be used to determine sequence identity or homology of a given protein, preferably using the BLOSUM 50 or PAM 250 scoring matrix, gap penalties of −12 and −2 and the PIR or SwissPROT databases for comparison and analysis purposes. The results are expressed as z values or E( ) values. To achieve a more “updated” z value cutoff for statistical significance, preferably corresponding to a z value >10 based on the increase in database size over that of 1988, in a FASTA analysis using the equivalent 2001 database, a significant z value would exceed 13.

A more widely used and preferred methodology determines the percent identity of two amino acid sequences or of two nucleic acid sequences after optimal alignment as discussed above, e.g., using BLAST. In a preferred embodiment of this approach, a polypeptide being analyzed for its homology with native protein is at least 20%, preferably at least 40%, more preferably at least 50%, even more preferably at least 60%, and even more preferably at least 70%, 80%, or 90% as long as the reference sequence. The amino acid residues (or nucleotides) at corresponding positions are then compared. Amino acid or nucleic acid “identity” is equivalent to amino acid or nucleic acid “homology”.

In a preferred comparison of a putative polypeptide or peptide homologue polypeptide and a native protein, the percent identity between two amino acid sequences is determined using the Needleman and Wunsch alignment algorithm (incorporated into the GAP program in the GCG software package using either a Blossom 62 matrix or a PAM250 matrix, and a gap weight of 16, 14, 12, 10, 8, 6, or 4 and a length weight of 1, 2, 3, 4, 5, or 6. In yet another embodiment, the percent identity between the encoding nucleotide sequences is determined using the GAP program in the GCG software package (also available at above URL), using a NWSgapdna. CMP matrix and a gap weight of 40, 50, 60, 70, or 80 and a length weight of 1, 2, 3, 4, 5, or 6. In another embodiment, the algorithm of Meyers et al., supra (incorporated into the ALIGN program, version 2.0), is implemented using a PAM 120 weight residue table, a gap length penalty of 12 and a gap penalty of 4.

The wild-type (or native) SAg-encoding nucleic acid sequence or the SAg protein sequence can further be used as a “query sequence” to search against a public database, for example, to identify other family members or related sequences. Such searches can be performed using the NBLAST and XBLAST programs, supra (see Altschul et al. (1990) J. Mol. Biol. 215:403-10). BLAST nucleotide searches can be performed with the NBLAST program, score=100, wordlength=12 to identify nucleotide sequences homologous to native SAgs. BLAST protein searches can be performed with the XBLAST program, score=50, wordlength=3 to identify amino acid sequences homologous to identify polypeptide molecules homologous to a native SAg. To obtain gapped alignments for comparison purposes, Gapped BLAST can be utilized as described in Altschul et al. (1997, supra). Default parameters of XBLAST and NBLAST are well established in the art.

Using the FASTA programs and method of Pearson and Lipman, a preferred SAg homologue is one that has a z value >10. Expressed in terms of sequence identity or similarity, a preferred SAg homologue for use according the present invention has at least about 20% identity or 25% similarity to native SAg. Preferred identity or similarity is higher. More preferably, the amino acid sequence of a homologue is substantially identical or substantially similar to a native protein molecule as those terms are defined above.

One group of substitution variants (also homologues) are those in which at least one amino acid residue in the peptide molecule, and preferably, only one, has been removed and a different residue inserted in its place. Deletion and addition variants are also homologues if they satisfy the structural and functional criteria set forth herein with respect to their parent or native molecules. For a detailed description of protein chemistry and structure, see Schulz, G. E. Principles of Protein Structure Springer-Verlag, New York, 1978, and Creighton, T. E., Proteins: Structure and Molecular Properties, W.H. Freeman & Co., San Francisco, 1983, which are hereby incorporated by reference. The types of substitutions which may be made in the protein or peptide molecule of the present invention may be based on analysis of the frequencies of amino acid changes between a homologous protein of different species, such as those presented in Table 1-2 of Schulz et al. (supra) and FIG. 3-9 of Creighton (supra). Based on such an analysis, conservative substitutions are defined herein as exchanges within one of the following five groups:

1. Small aliphatic, nonpolar or slightly polar residues: Ala, Ser, Thr (Pro, Gly);
2. Polar, negatively charged residues and their amides: Asp, Asn, Glu, Gln;
3. Polar, positively charged residues: His, kg, Lys;
4. Large aliphatic, nonpolar residues: Met, Leu, Ile, Val (Cys); and
5. Large aromatic residues: Phe, Tyr, Trp.

The three amino acid residues in parentheses above have special roles in protein architecture. Gly is the only residue lacking any side chain and thus imparts flexibility to the chain. Pro, because of its unusual geometry, tightly constrains the chain. Cys can participate in disulfide bond formation which is important in protein folding. Tyr, because of its hydrogen bonding potential, has some kinship with Ser, Thr, etc.

More substantial changes in functional or immunological properties are made by selecting substitutions that are less conservative, such as between, rather than within, the above five groups, which will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain. Examples of such substitutions are (a) substitution of gly and/or pro by another amino acid or deletion or insertion of Gly or Pro; (b) substitution of a hydrophilic residue, e.g., Ser or Thr, for (or by) a hydrophobic residue, e.g., Leu, Ile, Phe, Val or Ala; (c) substitution of a Cys residue for (or by) any other residue; (d) substitution of a residue having an electropositive side chain, e.g., Lys, Arg or His, for (or by) a residue having an electronegative charge, e.g., Glu or Asp; or (e) substitution of a residue having a bulky side chain, e.g., Phe, for (or by) a residue not having such a side chain, e.g., Gly.

The deletions and insertions, and substitutions according to the present invention are those which do not produce radical changes in the characteristics of the protein or peptide molecule. However, when it is difficult to predict the exact effect of the substitution, deletion, or insertion in advance of doing so, one skilled in the art will appreciate that the effect will be evaluated by routine screening assays, for example direct or competitive immunoassay of cytotoxicity or biological assay of T cell function as described herein. For non-superantigen homologues, the screening test(s) selected to assay function reflect the intrinsic functional activity of the native protein particularly its tumoricidal activity in the context of the inventions described herein. Modifications of such proteins or peptide properties as redox or thermal stability, hydrophobicity, susceptibility to proteolytic degradation or the tendency to aggregate with carriers or into multimers are assessed by methods well known to the ordinarily skilled artisan.

### Superantigen Homologues

The variants or homologues of native SAg proteins or peptides including mutants (substitution, deletion and addition types), fusion proteins (or conjugates) with other polypeptides, are characterized by substantial sequence homology to

(a) the long-known SE's—SEA, SEB, SEC1-3, SED, SEE and TSST-1;
(b) long-known SpE's;
(c) more recently discovered SE's (SEG, SEH, SEI, SEJ, SEK, SEL, SEM, SEN, SEO, SEP, SER, SEU, SETs 1-5); or
(d) non-enterotoxin superantigens (YPM, M. arthritides superantigen).

Preferred homologues were disclosed above.

Table 1 in PCT U.S. Ser. No. 05/022,638 filed Jun. 27, 2005 incorporated in its entirety by reference lists a number of native SEs and exemplary homologues (amino acid substitution, deletion and addition variants (mutants) and fragments) with z values >10 (range: z=16 to z=136) using the Lipman-Pearson algorithm and FASTA. These homologues also induce significant T lymphocyte mitogenic responses that are generally comparable to native SE's.

In addition, as shown in Table 2 of PCT U.S. Ser. No. 05/022,638 filed Jun. 27, 2005 incorporated in its entirety by reference, several of these homologues also promote antigen-nonspecific T lymphocyte killing in vitro by a mechanism termed “superantigen-dependent cellular cytotoxicity” (SDCC) or, in the case of SAg-mAb fusion proteins, “superantigen/antibody dependent cellular cytotoxicity (SADCC).”

According to the present invention, other SE homologues (e.g., z>10 or, in another embodiment, having at least about 20% sequence identity or at least about 25% sequence similarity when compared to native SEs), exhibiting T lymphocyte mitogenicity, SDCC or SADCC, are useful anti-tumor agents when administered to a tumor bearing host.

### Co-Stimulatory Ligands

The interaction with at least one co-stimulatory ligand provides a non-antigen-specific signal required for full activation of a T cell. Co-stimulatory ligands include B71 and B72 and tumor necrosis factor (TNF) ligands and cytokines (such as IL-2, IL-12, IL-15 or IL21), and immunoglobulin (Ig) superfamily ligands.

### TNF Ligands

Tumor necrosis factor (TNF) is a cytokine involved in systemic inflammation and stimulates the acute phase reaction. Its primary role is in the regulation of immune cells. Tumor necrosis factor (TNF) ligands share a number of common features. The majority of the ligands are synthesized as type II transmembrane proteins (extracellular C-terminus) containing a short cytoplasmic segment and a relatively long extracellular region. TNF ligands include, without limitation, nerve growth factor (NGF), CD40L (CD40L)/CD154, CD137L/4-1BBL, tumor necrosis factor alpha (TNFα), CD134L/OX40L/CD252, CD27L/CD70, Fas ligand (FasL), CD30L/CD153, tumor necrosis factor beta (TNFβ), lympho-toxin-alpha (LTα), lymphotoxin-beta (LTβ), CD257/B cell-activating factor (BAFF)/Blys/THANK/Tal1-1, glucocorticoid-induced TNF Receptor ligand (GITRL), and TNF-related apoptosis-inducing ligand (TRAIL), LIGHT (TNFSF14).

### Example 1

### Clinical Trial of PSSSRBCs or PS47SSRBCs Expressing and/or FAS and Heme

All patients treated have histologically confirmed malignant disease including carcinomas, sarcomas, melanomas, gliomas, neuroblastomas, lymphomas and leukemia and have failed conventional therapy. Patients may be diagnosed as having any stage of metastatic disease involving any organ system. Staging describes both tumor and host, including organ of origin of the tumor, histologic type and histologic grade, extent of tumor size, site of metastases and functional status of the patient. A general classification includes the known ranges of Stage I (localized disease) to Stage 4 (widespread metastases). Patient history is obtained and physical examination performed along with conventional tests of cardiovascular and pulmonary function and appropriate radiologic procedures. They have not been undergoing any other anticancer treatment for at least one month and have a clinical KPS of at least 50. Histopathology is obtained to verify malignant disease.

For human studies, SSRBCs expressing phophatidylserine are obtained from patients with homozygous S or sickle thalassemia hemoglobin, hemizygous sickle S and A hemoglobin, sickle hemoglobin-C disease, sickle beta plus thalassemia, sickle hemoglobin-D disease, sickle hemoglobin-E disease, homozygous C or C-thalassemia, hemoglobin-C beta plus thalassemia, homozygous E or E-thalassemia. Patient Evaluation: Assessment of response of the tumor to the therapy is made once per week during therapy and 30 days thereafter using CT or x-ray visualization. Depending on the response to treatment, side effects, and the health status of the patient, treatment is terminated or prolonged from the standard protocol given above. Tumor response criteria are those established by the WHO and RECIST (Response Evaluation Criteria in Solid Tumors) summarized below (also Abraham et al, supra).

SSRBC expressing PS are administered intravenously in volumes of 10-500 ml a period of 30 to 120 minutes. The treatments are generally given every 2-7 days for a total of 1-12 treatments. However, the treatment schedule is flexible and may be given for a longer of shorter duration depending upon the patients' response. The efficacy of the therapy in a patient population is evaluated using conventional statistical methods, including, for example, the Chi Square test or Fisher's exact test. Long-term changes in and short term changes in measurements are evaluated separately.

### Results

A total of 891 patients are patients treated. The number of patients for each tumor type and the results of treatment are summarized in Table 1 Positive tumor responses are observed in as high as 75-90%% of the patients with breast, gastrointestinal, lung, prostate, renal and bladder tumors as well as melanoma and neuroblastoma.

891 patients with all tumors exhibit objective clinical responses for an overall response rate of 86.9%. Tumors generally start to diminish and objective remissions are evident after four weeks of combined SAgs and chemotherapy. Responses endure for an average of 24 months.

Toxicity consists of mild short-lived fever, fatigue and anorexia not requiring treatment. The incidence of side effects (as % of total treatments) are as follows: chills—10; fever—10; pain—5; nausea—5; respiratory—3; headache—3; tachycardia—2; vomiting—2; hypertension—2; hypotension—2; joint pain—2; rash—2; flushing—1; diarrhea—1; itching/hives—1; bloody nose—1; dizziness—<1; cramps—<1; fatigue—<1; feeling faint—<1; twitching—<1; blurred vision—<1; gastritis<1; redness on hand—<1. Fever and chills are the most common side effects observed. CBC, renal and liver functions tests do not change significantly after treatments.

TABLE 1

| | | | % of Patients |
|---|---|---|---|
| Patients/Tumors | No. | Response | Responding |
| All Patients | 891 | CR + PR | 86.9 |
| Tumor Type | | | |
| Breast adenocarcinoma | 165 | CR + PR | 92% |
| Gastrointestinal carcinoma | 156 | CR + PR | 94% |
| Lung Carcinoma | 200 | CR + PR | 90% |
| Brain glioma/astrocytoma | 60 | CR + PR | 83% |
| Prostate Carcinoma | 130 | CR + PR | 87% |
| Lymphoma/Leukemia | 61 | CR + PR | 81% |
| Head and Neck Cancer | 82 | CR + PR | 80% |
| Renal and Bladder Cancer | 53 | CR + PR | 96% |
| Melanoma | 67 | CR + PR | 84% |
| Neuroblastoma | 37 | CR + PR | 82% |

### Example 2

### Clinical Trial

### PS47SCs Induce Tumoricidal Response

All patients treated have histologically confirmed malignant disease including carcinomas, sarcomas, melanomas, gliomas, neuroblastomas, lymphomas and leukemia and have failed conventional therapy. Patients may be diagnosed as having any stage of metastatic disease involving any organ system. Staging describes both tumor and host, including organ of origin of the tumor, histologic type and histologic grade, extent of tumor size, site of metastases and functional status of the patient. A general classification includes the known ranges of Stage I (localized disease) to Stage 4 (widespread metastases). Patient history is obtained and physical examination performed along with conventional tests of cardiovascular and pulmonary function and appropriate radiologic procedures. They have not been undergoing any other anticancer treatment for at least one month and have a clinical KPS of at least 50. Histopathology is obtained to verify malignant disease.

For human studies, PS47SCs are obtained from patients with homozygous S or sickle thalassemia hemoglobin, hemizygous sickle S and A hemoglobin, sickle hemoglobin-C disease, sickle beta plus thalassemia, sickle hemoglobin-D disease, sickle hemoglobin-E disease, homozygous C or C-thalassemia, hemoglobin-C beta plus thalassemia, homozygous E or E-thalassemia. Such cells are also derived from iPS erythroid cell obtained from these patients. Such cells from patients with homozygous S hemoglobin are preferred. Patient Evaluation:

Assessment of response of the tumor to the therapy is made once per week during therapy and 30 days thereafter using CT or x-ray visualization. Depending on the response to treatment, side effects, and the health status of the patient, treatment is terminated or prolonged from the standard protocol given above. Tumor response criteria are those established by the WHO and RECIST (Response Evaluation Criteria in Solid Tumors) summarized below (also Abraham et al, supra).

PS47SCs are administered intravenously in volumes of 10-500 ml a period of 30 to 120 minutes. The treatments are generally given every 2-7 days for a total of 1-12 treatments. However, the treatment schedule is flexible and may be given for a longer of shorter duration depending upon the patients' response. The efficacy of the therapy in a patient population is evaluated using conventional statistical methods, including, for example, the Chi Square test or Fisher's exact test. Long-term changes in and short term changes in measurements are evaluated separately.

### Results

A total of 1054 patients are patients are treated. The number of patients for each tumor type and the results of treatment are summarized in Table 1 Positive tumor responses are observed in as high as 75-90%% of the patients with breast, gastrointestinal, lung, prostate, renal and bladder tumors as well as melanoma and neuroblastoma.

1054 patients with all tumors exhibit objective clinical responses for an overall response rate of 86.7%. Tumors generally start to diminish and objective remissions are evident after four weeks of combined SAgs and chemotherapy. Responses endure for an average of 24 months.

Toxicity consists of mild short-lived fever, fatigue and anorexia not requiring treatment. The incidence of side effects (as % of total treatments) are as follows: chills—15; fever—11; pain—3; nausea—6; respiratory—7; headache—2; tachycardia—7; vomiting—9; hypertension—1; hypotension—3; joint pain—4; rash—2; flushing—1; diarrhea—1; itching/hives—2; bloody nose—1; dizziness—<1; cramps—<1; fatigue—<1; feeling faint—<1; twitching—<1; blurred vision—<1; gastritis<1; redness on hand—<1. Fever and chills are the most common side effects observed. CBC, renal and liver functions tests do not change significantly during or after treatments.

TABLE 1

| | | | % of Patients |
|---|---|---|---|
| Patients/Tumors | No. | Response | Responding |
| All Patients | 1054 | CR + PR | 86.7 |
| Tumor Type | | | |
| Breast adenocarcinoma | 171 | CR + PR | 94% |
| Gastrointestinal carcinoma | 150 | CR + PR | 90% |
| Lung Carcinoma | 190 | CR + PR | 92% |
| Brain glioma/astrocytoma | 71 | CR + PR | 83% |
| Prostate Carcinoma | 121 | CR + PR | 82% |
| Lymphoma/Leukemia | 73 | CR + PR | 82% |
| Head and Neck Cancer | 78 | CR + PR | 88% |
| Renal and Bladder Cancer | 81 | CR + PR | 95% |
| Melanoma | 76 | CR + PR | 81% |
| Neuroblastoma | 43 | CR + PR | 80% |

### Example 3

### Clinical Trial with Microparticles Shed from SSRBCs Expressing Phophatidylserine and/or FAS and Heme

All patients treated have histologically confirmed malignant disease including carcinomas, sarcomas, melanomas, gliomas, neuroblastomas, lymphomas and leukemia and have failed conventional therapy. Patients may be diagnosed as having any stage of metastatic disease involving any organ system. Staging describes both tumor and host, including organ of origin of the tumor, histologic type and histologic grade, extent of tumor size, site of metastases and functional status of the patient. A general classification includes the known ranges of Stage I (localized disease) to Stage 4 (widespread metastases). Patient history is obtained and physical examination performed along with conventional tests of cardiovascular and pulmonary function and appropriate radiologic procedures. They have not been undergoing any other anticancer treatment for at least one month and have a clinical KPS of at least 50. Histopathology is obtained to verify malignant disease.

For human studies, microparticles from SSRBCs expressing phophatidylserine are obtained from patients with homozygous S or sickle thalassemia hemoglobin, hemizygous sickle S and A hemoglobin, sickle hemoglobin-C disease, sickle beta plus thalassemia, sickle hemoglobin-D disease, sickle hemoglobin-E disease, homozygous C or C-thalassemia, hemoglobin-C beta plus thalassemia, homozygous E or E-thalassemia. Patient Evaluation: Assessment of response of the tumor to the therapy is made once per week during therapy and 30 days thereafter using CT or x-ray visualization. Depending on the response to treatment, side effects, and the health status of the patient, treatment is terminated or prolonged from the standard protocol given above. Tumor response criteria are those established by the WHO and RECIST (Response Evaluation Criteria in Solid Tumors) summarized below (also Abraham et al, supra).

Microparticle in amounts ranging from 2 to 250 ml are administered intravenously in volumes of 10-500 ml a period of 30 to 120 minutes. The treatments are generally given every 2-7 days for a total of 1-12 treatments. However, the treatment schedule is flexible and may be given for a longer of shorter duration depending upon the patients' response. The efficacy of the therapy in a patient population is evaluated using conventional statistical methods, including, for example, the Chi Square test or Fisher's exact test. Long-term changes in and short term changes in measurements are evaluated separately.

### Results

A total of 997 patients are patients treated. The number of patients for each tumor type and the results of treatment are summarized in Table 2. Positive tumor responses are observed in as high as 75-90%% of the patients with breast, gastrointestinal, lung, prostate, renal and bladder tumors as well as melanoma and neuroblastoma.

997 patients with all tumors exhibit objective clinical responses for an overall response rate of 88.3%. Tumors generally start to diminish and objective remissions are evident after four weeks of combined SAgs and chemotherapy. Responses endure for an average of 24 months.

Toxicity consists of mild short-lived fever, fatigue and anorexia not requiring treatment. The incidence of side effects (as % of total treatments) are as follows: chills—10; fever—10; pain—5; nausea—5; respiratory—3; headache—3; tachycardia—2; vomiting—2; hypertension—2; hypotension—2; joint pain—2; rash—2; flushing—1; diarrhea—1; itching/hives—1; bloody nose—1; dizziness—<1; cramps—<1; fatigue—<1; feeling faint—<1; twitching—<1; blurred vision—<1; gastritis<1; redness on hand—<1. Fever and chills are the most common side effects observed. CBC, renal and liver functions tests do not change significantly after treatments.

TABLE 2

| | | | % of Patients |
|---|---|---|---|
| Patients/Tumors | No. | Response | Responding |
| All Patients | 997 | CR + PR | 88.3 |
| Tumor Type | | | |
| Breast adenocarcinoma | 179 | CR + PR | 91% |
| Gastrointestinal carcinoma | 1170 | CR + PR | 93% |
| Lung Carcinoma | 214 | CR + PR | 95% |
| Brain glioma/astrocytoma | 84 | CR + PR | 81% |
| Prostate Carcinoma | 134 | CR + PR | 82% |
| Lymphoma/Leukemia | 75 | CR + PR | 87% |
| Head and Neck Cancer | 91 | CR + PR | 89% |
| Renal and Bladder Cancer | 562 | CR + PR | 91% |
| Melanoma | 69 | CR + PR | 86% |
| Neuroblastoma | 40 | CR + PR | 88% |

### Example 4

### Clinical Trial with Synthetic Microparticles Expressing Phophatidylserine and/or FAS and Heme

All patients treated have histologically confirmed malignant disease including carcinomas, sarcomas, melanomas, gliomas, neuroblastomas, lymphomas and leukemia and have failed conventional therapy. Patients may be diagnosed as having any stage of metastatic disease involving any organ system. Staging describes both tumor and host, including organ of origin of the tumor, histologic type and histologic grade, extent of tumor size, site of metastases and functional status of the patient. A general classification includes the known ranges of Stage I (localized disease) to Stage 4 (widespread metastases). Patient history is obtained and physical examination performed along with conventional tests of cardiovascular and pulmonary function and appropriate radiologic procedures. They have not been undergoing any other anticancer treatment for at least one month and have a clinical KPS of at least 50. Histopathology is obtained to verify malignant disease.

For human studies, microparticles from SSRBCs expressing phophatidylserine are obtained from patients with homozygous S or sickle thalassemia hemoglobin, hemizygous sickle S and A hemoglobin, sickle hemoglobin-C disease, sickle beta plus thalassemia, sickle hemoglobin-D disease, sickle hemoglobin-E disease, homozygous C or C-thalassemia, hemoglobin-C beta plus thalassemia, homozygous E or E-thalassemia. Patient Evaluation:

Assessment of response of the tumor to the therapy is made once per week during therapy and 30 days thereafter using CT or x-ray visualization. Depending on the response to treatment, side effects, and the health status of the patient, treatment is terminated or prolonged from the standard protocol given above. Tumor response criteria are those established by the WHO and RECIST (Response Evaluation Criteria in Solid Tumors).

Microparticle in amounts ranging from 2 to 250 ml are administered intravenously in volumes of 10-500 ml a period of 30 to 120 minutes. The treatments are generally given every 2-7 days for a total of 1-12 treatments. However, the treatment schedule is flexible and may be given for a longer of shorter duration depending upon the patients' response. The efficacy of the therapy in a patient population is evaluated using conventional statistical methods, including, for example, the Chi Square test or Fisher's exact test. Long-term changes in and short term changes in measurements are evaluated separately.

### Results

A total of 1041 patients are patients treated. The number of patients for each tumor type and the results of treatment are summarized in Table 3. Positive tumor responses are observed in as high as 75-90%% of the patients with breast, gastrointestinal, lung, prostate, renal and bladder tumors as well as melanoma and neuroblastoma.

1041 patients with all tumors exhibit objective clinical responses for an overall response rate of 87.4%. Tumors generally start to diminish and objective remissions are evident after four weeks of combined SAgs and chemotherapy. Responses endure for an average of 24 months.

Toxicity consists of mild short-lived fever, fatigue and anorexia not requiring treatment. The incidence of side effects (as % of total treatments) are as follows: chills—10; fever—10; pain—5; nausea—5; respiratory—3; headache—3; tachycardia—2; vomiting—2; hypertension—2; hypotension—2; joint pain—2; rash—2; flushing—1; diarrhea—1; itching/hives—1; bloody nose—1; dizziness—<1; cramps—<1; fatigue—<1; feeling faint—<1; twitching—<1; blurred vision—<1; gastritis<1; redness on hand—<1. Fever and chills are the most common side effects observed. CBC, renal and liver functions tests do not change significantly after treatments.

TABLE 3

| | | | % of Patients |
|---|---|---|---|
| Patients/Tumors | No. | Response | Responding |
| All Patients | 1041 | CR + PR | 87.4 |
| Tumor Type | | | |
| Breast adenocarcinoma | 178 | CR +PR | 90% |
| Gastrointestinal carcinoma | 174 | CR + PR | 96% |
| Lung Carcinoma | 218 | CR + PR | 92% |
| Brain glioma/astrocytoma | 83 | CR + PR | 83% |
| Prostate Carcinoma | 133 | CR + PR | 87% |
| Lymphoma/Leukemia | 74 | CR + PR | 81% |
| Head and Neck Cancer | 94 | CR + PR | 84% |
| Renal and Bladder Cancer | 561 | CR + PR | 96% |
| Melanoma | 71 | CR + PR | 83% |
| Neuroblastoma | 42 | CR + PR | 82% |

### Example 5

### Robust Autoexpansion of Cytotoxic T Cell Population with Specificity for Tumor Receptors by Co-Transfected with SAg, MHCII and Tumor Receptor Ligand

Expression of transduced SAg and MHCII ligands in T cells leads to autostimulation of human primary T cells. Peripheral blood T lymphocyte proliferation by T cells transduced with SAg/MHCII are quantified and compared with T cells transduced with either molecule alone or none. T cells transduced with MHCII alone failed to proliferate whereas T cells transduced with SAg alone spontaneously proliferate to 45 fold above baseline. In sharp contrast, T cells transduced with SAg and MHCII showed a 400 fold greater proliferation over 21 days (p<0.0001).

In another method of rapidly generate tumor-reactive human T lymphocytes, peripheral blood T cells are retrovirally transduced with the EGF (Zhou et al., PLOS ONE 6 (2): e16642. doi: 10.1371 (2011) or the chimeric antigen receptor Pzl (Gade et al., Cancer Res. 65:9080-9088 (2005), a non-HLA-restricted antigen receptor specific for the tumor antigen PSMA on prostate carcinoma cells. The Pzl receptor comprises a PSMA-binding single chain antibody fragment fused to the human CD3 zeta signaling domain. EGF or Pz1⁺ T cells coexpressing SAg and MHCII mount a robust autoproliferative response (mean 1042-fold enrichment). This expansion is 12-fold, greater (p<0.0001) than that obtained when such T cells express SAg or MHCII or the EGF or PZI on the tumor cell versus the T cells. Further analyses documents reduced production of IL-2 and IFN-γ by exposure of T cells transduced with SEG, MHCII than T cells transduced with SEA and MHCII. These T cells also showed greater tumor specific-specific cytolytic activity and decreased susceptibility to apoptosis, when compared to non-transduced T lymphocytes. In aggregate, these in vitro studies demonstrate the ability of T lymphocytes coexpressing SAg and MHCII cells to strongly augment suboptimal TCR-activation to produce a population of tumor specific cytotoxic tumor cells.

### Example 6

### T Cells Co-Expressing SAg and MHCII Eradicate Established, Systemic Tumors

To investigate the potency of our SAg-MHCII T cells in vivo, models of multifocal, established EGF expressing breast cancer or prostate cancer expressing the PSMA-PC-3 antigen using the EGF expressing or PZI expressing T cells respectively are used (Gong et al., Neoplasia 1:123-7, 1999 which is hereby incorporated by reference in its entirety).

In these models, animals are treated four weeks after tumor inoculation with a single intravenous infusion of EGF- or 8×10⁶ PSMA-targeted T cells, expressing SEG and MHCII alpha chain. Control mice treated untransduced cytotoxic CD8+ T lymphocytes or CD8+ cytotoxic T cell transduced with SAg and alpha chain devoid of a tumor targeting moiety results in a short-term reduction of tumor burden, followed by terminal tumor progression yielding a modest 12-day survival advantage. Similar results were obtained with EGF tumor bearing mice treated with T cells transduced with EGF alone. Cytotoxic T cells engineered to express SEG, MHCII alpha chain, fv specific for PSMA or EGF show complete tumor regressions and survival exceeding 100 days (P<0.000001) in each model.

These self-stimulating cytotoxic T lymphocytes are tested in the animal models described “Tumor Models and Procedures for Evaluating Anti-Tumor Effects Studies” with similar statistically significant results.

### Example 7

### Clinical Trial of TCD47Es in Human Cancer Patients

TCD47Es consists of erythroid progenitor cells transduced with cDNA extracted from untreated tumor cells, treatment-resistant tumor cells or normal cells of the same histologic type as the tumor. Prior to DNA extraction such untreated tumor cells, treatment-resistant tumor cells or normal cells are initially transduced with a self-transforming virus such a VSV. The TC47Es are tested for therapeutic efficacy in human cancer patients.

All patients treated have histologically confirmed malignant masses confirmed by biopsy or cytology. Malignant diseases including carcinomas, sarcomas, melanomas, gliomas neuroblastomas, lymphomas and leukemia. The malignant disease has failed to respond or is advancing despite conventional therapy. Patients in all stages of malignant disease involving any organ system are included. Staging describes both tumor and host, including organ of origin of the tumor, histologic type, histologic grade, extent of tumor size, site of metastases and functional status of the patient. For a general classification includes the known ranges of Stage 1 (localized disease) to Stage 4 (widespread metastases), see Abraham J et al., Bethesda Handbook of Clinical Oncology, Lippincott, Williams & Wilkins, Philadelphia, Pa., 2001. Patient history is obtained and physical examination performed along with conventional tests of cardiovascular and pulmonary function and appropriate radiologic procedures. The malignant masses are visible on x-ray or CT scan and are measurable with calipers. They have not been undergoing any other anticancer treatment for at least one month and have a clinical KPS of at least 50.

For primary tumors, the TC47Es derived from DNA extracted from primary tumor cells are administered parenterally preferably intravenously or intraperitoneally by infusion or injection once weekly in doses of 10⁸ cells for up to 5 injections. Likewise, metastatic tumors are treated with TC47Es derived from DNA extracted from metastatic tumor cells in a similar fashion. In both instances, TCD47Es derived from DNA extracted from normal tissue cells are used once weekly for up to 5 injections. Patients with both primary and metastatic tumors are treated with TC47E derived from DNA extracted from both primary and metastatic tumor cells once weekly for 5 weeks.

### Patient Evaluation:

Assessment of response of the tumor to the therapy is made once per week during therapy and 30 days thereafter using CT or x-ray visualization. Depending on the response to treatment, side effects, and the health status of the patient, treatment is terminated or prolonged from the standard protocol given above. Tumor response criteria are those established by the WHO and RECIST (Response Evaluation Criteria in Solid Tumors) summarized below in 10 Table 6 (also Abraham et al., supra).

Response Definition
Complete remission Disappearance of all evidence of disease (CR)
Partial remission (PR) >50% decrease in the product of the two greatest perpendicular tumor diameters; no new lesions
Less than partial 25%-50% decrease in tumor size, stable for at least 1 month remission (<PR)
Stable disease <25% reduction in tumor size; no progression or new lesions
Progression >25% increase in size of any one measured lesion or appearance of new lesions despite stabilization or remission of disease in other measured sites

The efficacy of the therapy in a patient population is evaluated using conventional statistical methods, including, for example, the Chi Square test or Fisher's exact test. Long-term changes in and short term changes in measurements are evaluated separately.

### Results

A total of 1058 patients are patients treated. The number of patients for each tumor type and the results of treatment are summarized in Table. Positive tumor responses are observed in 76-89% of the patients with breast, gastrointestinal, lung, prostate, renal and bladder tumors as well as melanoma and neuroblastoma as follows Tumors generally start to diminish and objective remissions are evident after four weeks of combined SEA-chemotherapy. Responses endure for an average of 24 months.

### Toxicity

Toxicity consists of mild short-lived fever, fatigue and anorexia not requiring treatment. The incidence of side effects (as % of total treatments) are as follows: chills—14; fever—12; pain—4; nausea—3; respiratory—1; headache—2; tachycardia—3 vomiting—5; hypertension—1; hypotension—3; joint pain—1; rash—2; flushing—7; diarrhea—2; itching/hives—6; bloody nose—1; dizziness—<1; cramps—<1; fatigue—<1; feeling faint—<1; twitching—<1; blurred vision—<1; gastritis<1; redness on hand—<1. Fever and chills are the most common side effects observed. CBC, kidney and liver functions tests do not change significantly after treatments.

TABLE 4

| | | | % of Patients |
|---|---|---|---|
| Tumor Type: | No. | Response | Responding |
| Breast adenocarcinoma | 131 | CR + PR + <PR | 82% |
| Gastrointestinal carcinoma | 102 | CR + PR + <PR | 85% |
| Lung Carcinoma | 195 | CR + PR + <PR | 89% |
| Brain glioma/astrocytoma | 70 | CR + PR + <PR | 80% |
| Prostate Carcinoma | 120 | CR + PR + <PR | 79% |
| Lymphoma/Leukemia | 103 | CR + PR + <PR | 76% |
| Head and Neck Cancer | 95 | CR + PR + <PR | 81% |
| Renal and Bladder Cancer | 71 | CR + PR + <PR | 88% |
| Melanoma | 83 | CR + PR + <PR | 84% |
| Neuroblastoma | 88 | CR + PR + <PR | 86% |

### Example 8

### Clinical Trial Using T Cells Activated by T47EPS-Stimulated Macrophages

TCD47Es consists of erythroid progenitor cells transduced with cDNA extracted from untreated tumor cells, treatment-resistant tumor cells or normal cells of the same histologic type as the tumor. Prior to DNA extraction such untreated tumor cells, treatment-resistant tumor cells or normal cells are initially transduced with a self-transforming virus such a VSV. The TC47Es are tested for therapeutic efficacy in human cancer patients. The current trial is designed to test T cells that have been immunized by TCD47Es in vivo and in vitro for anti-tumor efficacy.

Cohort 1 receives T cells collected from patients immunized with CD47Es in vivo. Five to twenty days thereafter, T cells are harvested from such patients, expanded further in vitro with anti-CD3/anti-CD47, harvested and redelivered to tumor bearing patients.

Cohort 2 receives T cells activated by TC47E stimulated macrophages. The macrophages polarized by TC47E exposure present their broad tumor antigen library to T cells in vitro. The latter are harvested, expanded further in vitro and used to adoptive therapy of human cancer patients.

In both cohorts, all patients treated have histologically confirmed malignant masses confirmed by biopsy or cytology. Malignant diseases including carcinomas, sarcomas, melanomas, gliomas neuroblastomas, lymphomas and leukemia. The malignant disease has failed to respond or is advancing despite conventional therapy. Patients in all stages of malignant disease involving any organ system are included. Staging describes both tumor and host, including organ of origin of the tumor, histologic type, histologic grade, extent of tumor size, site of metastases and functional status of the patient. For a general classification includes the known ranges of Stage 1 (localized disease) to Stage 4 (widespread metastases), see Abraham J et al., Bethesda Handbook of Clinical Oncology, Lippincott, Williams & Wilkins, Philadelphia, Pa., 2001. Patient history is obtained and physical examination performed along with conventional tests of cardiovascular and pulmonary function and appropriate radiologic procedures. The malignant masses are visible on x-ray or CT scan and are measurable with calipers. They have not been undergoing any other anticancer treatment for at least one month and have a clinical KPS of at least 50.

For primary tumors, the TC47Es derived from DNA extracted from primary tumor cells are administered parenterally preferably intravenously or intraperitoneally by infusion or injection once weekly in doses of 10⁸ cells for up to 5 injections. Likewise, metastatic tumors are treated with TC47Es derived from DNA extracted from metastatic tumor cells in a similar fashion. In both instances, TCD47Es derived from DNA extracted from normal tissue cells are used once weekly for up to 5 injections. Patients with both primary and metastatic tumors are treated with TC47E derived from DNA extracted from both primary and metastatic tumor cells once weekly for 5 weeks.

### Patient Evaluation:

Assessment of response of the tumor to the therapy is made once per week during therapy and 30 days thereafter using CT or x-ray visualization. Depending on the response to treatment, side effects, and the health status of the patient, treatment is terminated or prolonged from the standard protocol given above. Tumor response criteria are those established by the WHO and RECIST (Response Evaluation Criteria in Solid Tumors) as provided in Example 4. The efficacy of the therapy in a patient population is evaluated using conventional statistical methods, including, for example, the Chi Square test or Fisher's exact test. Long-term changes in and short term changes in measurements are evaluated separately.

### Results

In cohort 1 A total of 1167 patients are patients treated and in cohort 2 a total of 1231 patients are treated. The number of patients for each tumor type and the results of treatment are summarized in Table. Positive tumor responses are observed in as high as 75-89%% of the patients with breast, gastrointestinal, lung, prostate, renal and bladder tumors as well as melanoma and neuroblastoma as follows: Tumors generally start to diminish and objective remissions are evident after four weeks of combined SEA-chemotherapy. Responses endure for an average of 24 months.

### Toxicity

Toxicity consists of mild short-lived fever, fatigue and anorexia not requiring treatment. There is no stage III or stage IV toxicity. CBC, kidney and liver functions tests do not change significantly after treatments.

TABLE 5
Cohort 1

| | | | % of Patients |
|---|---|---|---|
| Tumor Type: | No. | Response | Responding |
| Breast adenocarcinoma | 142 | CR + PR + <PR | 86% |
| Gastrointestinal carcinoma | 111 | CR + PR + <PR | 82% |
| Lung Carcinoma | 204 | CR + PR + <PR | 87% |
| Brain glioma/astrocytoma | 81 | CR + PR + <PR | 89% |
| Prostate Carcinoma | 134 | CR + PR + <PR | 77% |
| Lymphoma/Leukemia | 115 | CR + PR + <PR | 75% |
| Head and Neck Cancer | 106 | CR + PR + <PR | 82% |
| Renal and Bladder Cancer | 81 | CR + PR + <PR | 86% |
| Melanoma | 96 | CR + PR + <PR | 85% |
| Neuroblastoma | 97 | CR + PR + <PR | 81% |

TABLE 6
Cohort 2

| | | | % of Patients |
|---|---|---|---|
| Tumor Type: | No. | Response | Responding |
| Breast adenocarcinoma | 155 | CR + PR + <PR | 86% |
| Gastrointestinal carcinoma | 122 | CR + PR + <PR | 87% |
| Lung Carcinoma | 207 | CR + PR + <PR | 81% |
| Brain glioma/astrocytoma | 83 | CR + PR + <PR | 85% |
| Prostate Carcinoma | 147 | CR + PR + <PR | 78% |
| Lymphoma/Leukemia | 121 | CR + PR + <PR | 78% |
| Head and Neck Cancer | 117 | CR + PR + <PR | 83% |
| Renal and Bladder Cancer | 87 | CR + PR + <PR | 87% |
| Melanoma | 93 | CR + PR + <PR | 82% |
| Neuroblastoma | 99 | CR + PR + <PR | 84% |

### Example 9

### Clinical Trial of PSSSRBCs or PS47SSRBCs with Chemotherapy

All patients treated have histologically confirmed malignant disease including carcinomas, sarcomas, melanomas, gliomas, neuroblastomas, lymphomas and leukemia and have failed conventional therapy. Patients may be diagnosed as having any stage of metastatic disease involving any organ system. Staging describes both tumor and host, including organ of origin of the tumor, histologic type and histologic grade, extent of tumor size, site of metastases and functional status of the patient. A general classification includes the known ranges of Stage I (localized disease) to Stage 4 (widespread metastases). Patient history is obtained and physical examination performed along with conventional tests of cardiovascular and pulmonary function and appropriate radiologic procedures. They have not been undergoing any other anticancer treatment for at least one month and have a clinical KPS of at least 50. Histopathology is obtained to verify malignant disease.

For human studies, PSSSRBCs or PS47SSRBCs are obtained from patients with homozygous S or sickle thalassemia hemoglobin, hemizygous sickle S and A hemoglobin, sickle hemoglobin-C disease, sickle beta plus thalassemia, sickle hemoglobin-D disease, sickle hemoglobin-E disease, homozygous C or C-thalassemia, hemoglobin-C beta plus thalassemia, homozygous E or E-thalassemia. Patient Evaluation: Assessment of response of the tumor to the therapy is made once per week during therapy and 30 days thereafter using CT or x-ray visualization. Depending on the response to treatment, side effects, and the health status of the patient, treatment is terminated or prolonged from the standard protocol given above. Tumor response criteria are those established by the WHO and RECIST (Response Evaluation Criteria in Solid Tumors) summarized below (also Abraham et al, supra).

SSRBC expressing PS are administered intravenously in volumes of 10-500 ml a period of 30 to 120 minutes. The treatments are generally given every 2-7 days for a total of 1-12 treatments. However, the treatment schedule is flexible and may be given for a longer of shorter duration depending upon the patients' response. The efficacy of the therapy in a patient population is evaluated using conventional statistical methods, including, for example, the Chi Square test or Fisher's exact test. Long-term changes in and short term changes in measurements are evaluated separately. Chemotherapy with gemcitabine (1000 mg/m²) given intravenously on day 1, 2, 3, or 4 prior to the infusion of PSSSRBCs or PS47SSRBCs or on day 1, 2, 3, 4 or 5 after infusion. In a second cohort, doxorubicin (75 mg/m²) is given intravenously on day 1, 2, 3, or 4 prior to the infusion of PSSSRBCs or PS47SSRBCs or on day 1, 2, 3, 4 or 5 after infusion. Other chemotherapeutic agents provided in the Bethesda Handbook of Clinical Oncology, 4th edition, Abraham J et al., Wolters Kluwer/Lippincott William & Wilkins, Philadelphia, Pa. (2014) are also useful in full therapeutic dosages before, during or after infusion of PSSSRBCs or PS47SSRBCs including but not limited to cisplatin (100 mg/m² i.v.), docetaxel (100 mg/m² i.v.), topotecan (1.5 mg/m² i.v. for 5 consecutive days).

### Results

A total of 1418 patients are treated PSSSRBCs or PS47SSRBCs plus gemcitabine. A total of 1593 are treated PSSSRBCs or PS47SSRBCs plus doxorubicin. The number of patients for each tumor type and the results of treatment are summarized in Tables 7 and 8. Positive tumor responses range from 80-98% of the patients with breast, gastrointestinal, lung, prostate, renal and bladder tumors as well as melanoma and neuroblastoma.

Eighty six percent of patients with all tumors exhibit objective clinical responses for an overall response rate of 86.6%. Tumors generally start to diminish and objective remissions are evident after four weeks of combined SAgs and chemotherapy. Responses endure for an average of 24 months.

Toxicity consists of mild short-lived fever, fatigue and anorexia not requiring treatment. The incidence of side effects (as % of total treatments) are as follows: chills—12; fever—15; pain—8; nausea—9; respiratory—5; headache—8; tachycardia—4; vomiting—5; hypertension—7; hypotension—6; joint pain—1; rash—3; flushing—2; diarrhea—2; itching/hives—1; bloody nose—1; dizziness—<1; cramps—<1; fatigue—<1; feeling faint—<1; twitching—<1; blurred vision—<1; gastritis<1; redness on hand—<1. Fever and chills are the most common side effects observed. CBC, renal and liver functions tests do not change significantly after treatments.

TABLE 7
PSSSRBCs or PS47SSRBCs with gemcitabine

| | | | % of Patients |
|---|---|---|---|
| Patients/Tumors | No. | Response | Responding |
| All Patients | 1418 | CR + PR | 86.6% |
| Tumor Type | | | |
| Breast adenocarcinoma | 205 | CR + PR | 88% |
| Gastrointestinal carcinoma | 191 | CR + PR | 91% |
| Lung Carcinoma | 283 | CR + PR | 86% |
| Brain glioma/astrocytoma | 94 | CR + PR | 87% |
| Prostate Carcinoma | 201 | CR + PR | 89% |
| Lymphoma/Leukemia | 105 | CR + PR | 80% |
| Head and Neck Cancer | 110 | CR + PR | 84% |
| Renal and Bladder Cancer | 81 | CR + PR | 98% |
| Melanoma | 97 | CR + PR | 82% |
| Neuroblastoma | 51 | CR + PR | 81% |

TABLE 8
PSSSRBCs or PS47SSRBCs with doxorubicin

| | | | % of Patients |
|---|---|---|---|
| Patients/Tumors | No. | Response | Responding |
| All Patients | 1593 | CR + PR | 86.3% |
| Tumor Type | | | |
| Breast adenocarcinoma | 245 | CR + PR | 91% |
| Gastrointestinal carcinoma | 187 | CR + PR | 87% |
| Lung Carcinoma | 301 | CR + PR | 85% |
| Brain glioma/astrocytoma | 106 | CR + PR | 88% |
| Prostate Carcinoma | 216 | CR + PR | 86% |
| Lymphoma/Leukemia | 127 | CR + PR | 82% |
| Head and Neck Cancer | 134 | CR + PR | 85% |
| Renal and Bladder Cancer | 113 | CR + PR | 96% |
| Melanoma | 101 | CR + PR | 80% |
| Neuroblastoma | 63 | CR + PR | 83% |

All the references patents and patent application cited above in this patent application including those below and their references are incorporated by reference in entirety. Whether specifically incorporated or not. In addition, the following patent applications and their references are incorporated by reference in entirety with their references.

| Inventor | Ser. No. | Filing Date | Title |
|---|---|---|---|
| Terman, D. S. | 62/133,684 | Mar. 16, 2015 | Tumor Targeted Erythrocytes, Microparticles & Liposome for |
| | | | Treatment of Cancer |
| Terman, D. S. | 62/054,231 | Sep. 23, 2013 | Tumor Targeted Erythrocytes, Microparticles & Liposome for |
| | | | Treatment of Cancer |
| Terman, D. S | 62/025,396 | Jul. 16, 2014 | Sickle Erythrocytes and Progenitors Target Cytotoxics to Tumors |
| Terman, D. S. | 14/222,292 | Mar. 21, 2014 | Sickle Erythrocytes and Progenitors Target Cytotoxics to Tumors |
| Terman, D. S. | 13/328,748 | Dec. 16, 2011 | Compositions and Methods for Treatment of Cancer |
| Terman, D. S. | 14,037,176 | Sep. 25, 2013 | Compositions and Methods for Treatment of Cancer |
| Terman, D. S. | 61,807,457 | Apr. 2, 2013 | Sickled Erythrocytes Alone or with Anti-tumor Agents Induce Tumor |
| | | | Vaso-occlusion and Tumoricidal Effects |
| Terman, D. S. | 13/367,797 | Feb. 7, 2012 | Sickled Erythrocytes with Anti-tumor Agents Induce Tumor Vaso- |
| | | | occlusion and Tumoricidal Effects |
| Terman, D. S. | 13/317,590 | Oct. 20, 2011 | Compositions and Methods for Treatment of Cancer |
| Terman, D. S. | 61/455,592 | Oct. 20, 2010 | Compositions and Methods for Treatment of Cancer |
| Terman, D. S. | 12/586,532 | Sep. 22, 2009 | Sickled Erythrocytes with Anti-tumor Agents Induce Tumor |
| | | | Vaso-occlusion and Tumoricidal Effects |
| Terman D. S. | 12/276,941 | Nov. 24, 2008 | Compositions and Methods for Treatment of Cancer |
| Terman D. S. | 12/145,949 | Jun. 25, 2008 | Compositions and Methods for Treatment of Cancer |
| Terman D. S. | 10/937,758 | Sep. 8, 2004 | Compositions and Methods for Treatment of Cancer |
| Terman, D. S. | 61,215,906 | May 11, 2009 | Sickled Erythrocytes, Nucleated Precursors & Erythroleukemia Cells |
| | | | for Targeted Delivery of Tumoricidal Agents |
| Terman, D. S | 61/211,227 | Mar. 28, 2009 | Sickled Erythrocytes, Nucleated Precursors & Erythroleukemia Cells |
| | | | for Targeted Delivery of Tumoricidal Agents |
| Terman, D. S. | 61/206.338 | Jan. 28, 2009 | Sickled Erythrocytes, Nucleated Precursors & Erythroleukemia Cells |
| | | | for Targeted Delivery of Tumoricidal Agents |
| Terman D. S. | 61/205,776 | Jan. 22, 2009 | Sickled Erythrocytes Induced Tumor Vaso-occlusion and |
| | | | Tumoricidal Effects |
| Terman, D. S. | 61/192,949 | Sep. 22, 2008 | Sickled Erythrocytes, Nucleated Precursors & Erythroleukemia Cells |
| | | | for Targeted Delivery of Oncolytic Viruses, Anti-tumor Proteins, |
| | | | Plasmids, Toxins, Hemolysins and Chemotherapy |
| Terman, D, S, | PCT/US07/69869 | May 29, 2007 | Sickled Erythrocytes, Nucleated Precursors & Erythroleukemia Cells |
| Dewhirst M. W. | | | for Targeted Delivery of Oncolytic Viruses, Anti-tumor Proteins, |
| | | | Plasmids, Toxins, Hemolysins and Chemotherapy |
| Terman, D. S. | 60/842,213 | Sep. 5, 2006 | Sickled Erythrocytes & Nucleated Precursors for Targeted Delivery |
| | | | of Oncolytic Toxins, Viruses, hemolysins and chemotherapy |
| Terman, D. S. | 60/819,551 | Jul. 8, 2006 | Sickled Erythrocytes & Nucleated Precursors for Targeted Delivery |
| | | | of Oncolytic Toxins, Viruses, hemolysins and chemotherapy |
| Terman, D. S. | 60/809,553 | May 30, 2006 | Sickled Erythrocytes & Nucleated Precursors for Targeted Delivery |
| | | | of Oncolytic Toxins, Viruses, hemolysins and chemotherapy |
| Terman, D. S. | 60/799514 | May 10, 2006 | Synergy of Superantigens, Cytokines and Chemotherapy in |
| Bohach, G | | | Treatment of Malignant Disease |
| Terman, D. S, Etiene, J., | PCTUS05/022638 | Jun. 27, 2005 | Enterotoxin Gene Cluster Superantigens (egc) to Treat Malignant |
| Vandenesch, F., | | | Disease |
| Lina, G. Bohach, G. | | | |
| Terman, D. S, Etiene, J., | 60/583,692 | Jun. 29, 2004 | Enterotoxin Gene Cluster Superantigens (egc) to Treat Malignant |
| Vandenesch, F., | | | Disease |
| Lina, G. Bohach, G. | | | |
| Terman, D. S. | 60/665,654 | Mar. 23, 2005 | Enterotoxin Gene Cluster Superantigens (egc) to Treat Malignant |
| | | | Disease |
| Terman, D. S, Etiene, J., | 60/626,159 | Nov. 6, 2004 | Enterotoxin Gene Cluster Superantigens (egc) to Treat Malignant |
| Vandenesch, F., | | | Disease |
| Lina, G. Bohach, G. | | | |
| Terman, D. S. | 60/583,692 | Jun. 29, 2004 | Intrathecal and Intrapleural Superantigens to Treat Malignant |
| | | | Disease |
| Terman, D. S. | 60/550,926 | Mar. 5, 2004 | Intrathecal and Intrapleural Superantigens to Treat Malignant |
| | | | Disease |
| Terman, D. S. | 60/539,863 | Jan. 27, 2004 | Intrathecal and Intrapleural Superantigens to Treat Malignant |
| | | | Disease |
| Terman, D. S. | PCT/US03/14381 | May 8, 2003 | Intrathecal and Intrapleural Superantigens to Treat Malignant |
| | | | Disease |
| Terman, D. S. | 10/428,817 | May 5, 2003 | Composition and Methods for Treatment of Neoplastic Diseases |
| Terman, D. S. | 60/438,686 | Jan. 9, 2003 | Intrathecal and Intrapleural Superantigens to Treat Malignant |
| | | | Disease |
| Terman, D. S. | 60/415,310 | Oct. 1, 2002 | Intrathecal and Intratumoral Superantigens to Treat Malignant |
| | | | Disease. |
| Terman, D. S. | 60/406,750 | Aug. 29, 2002 | Intrathecal Superantigens to Treat Malignant Fluid Accumulation |
| Terman, D. S. | 60/415,400 | Oct. 2, 2002 | Composition and Methods for Treatment of Neoplastic Diseases |
| Terman, D. S. | 60/406,697 | Aug. 28, 2002 | Compositions and Methods for Treatment of Neoplastic Diseases |
| Terman, D. S. | 60/389,366 | Jun. 15, 2002 | Compositions and Methods for Treatment of Neoplastic Diseases |
| Terman, D. S. | 60/378,988 | May 8, 2002 | Compositions and Methods for Treatment of Neoplastic Diseases |
| Terman, D. S. | 09/870,759 | May 30, 2001 | Compositions and Methods for Treatment of Neoplastic Diseases |
| Terman, D. S. | 09/751,708 | Dec. 28, 2000 | Compositions and Methods for Treatment of Neoplastic Diseases |
| Terman, D. S. | 09/640,884 | Aug. 30, 2000 | Compositions and Methods for Treatment of Neoplastic Diseases |
| Terman, D. S. | 60/151,470 | Aug. 30, 1999 | Compositions and Methods for Treatment of Neoplastic Diseases |

Having now fully described this invention, it will be appreciated by those skilled in the art that the same can be performed within a wide range of equivalent parameters, concentrations, and conditions without departing from the spirit and scope of the invention and without undue experimentation.

1. A method of treating a subject with a tumor comprising the steps of:
(i) transducing a tumor cell or a normal cell or and- a treatment resistant tumor cell of the same histologic type with a virus or its genomic DNA, and
(ii) extracting the DNA individually from each transduced tumor cell or normal cell or treatment resistant tumor cell, and
(iii) incorporating each said individually extracted DNA into a virus or its genomic DNA and
(iv) transducing individual CD47 deficient erythroblast populations with said individually extracted DNA incorporated in a virus or its genomic DNA
(iv) administering to said subject in need thereof parenterally by infusion or injection a tumoricidally effective amount of at least one of said transduced CD47 deficient erythroblast populations.

2. A method of treating a subject with a tumor comprising the steps of:
(i) transducing a tumor cell or a normal cell or and- a treatment resistant tumor cell of the same histologic type with a virus or its genomic DNA, and
(ii) extracting the DNA individually from each transduced tumor cell or normal cell or treatment resistant tumor cell, and
(iii) incorporating each said individually extracted DNA into a virus or its genomic DNA and
(iv) transducing individual CD47 deficient erythroblast populations with said individually extracted DNA incorporated in a virus or its genomic DNA and
(iv) administering to said subject in need thereof parenterally by infusion or injection a tumoricidally effective amount of at least one of said transduced CD47 deficient erythroblast populations,
(vi) collecting T cells from subject receiving said transduced CD47 transduced erythroblast populations and
(vii) expanding and enriching said T cells in vitro and
(viii) administering a tumoricidally effective amount of at least one T cell population.

3. A method of treating a subject with a tumor comprising the steps of:
(i) transducing a tumor cell or a normal cell or a treatment resistant tumor cell of the same histologic type with a virus or its genomic DNA, and
(ii) extracting the DNA individually from each transduced tumor cell or normal cell or treatment resistant tumor cell expressing tumor associated antigens and
(iii) incorporating each said individually extracted DNA into a virus or its genomic DNA and
(iv) transducing individual CD47 deficient erythroblast populations with said individually extracted DNA incorporated in a virus or its genomic DNA
(v) incubating said CD47 deficient erythroblast populations with macrophages to produce macrophages expressing tumor associated antigens,
(vi) incubating said macrophages expressing tumor associated antigens with T cells to produce T cells immunized against tumor associated antigens expressed on said macrophages,
(vi) administering to said subject in need thereof parenterally by infusion or injection a tumoricidally effective amount of at least one of said T cell populations.

4. A method of treating a subject with a tumor comprising the steps of:
(i) transducing a tumor cell or a normal cell or and- a treatment resistant tumor cell of the same histologic type with a virus or its genomic DNA, and
(ii) extracting the DNA individually from each transduced tumor cell or normal cell or treatment resistant tumor cell expressing tumor associated antigens, and
(iii) incorporating each said individually extracted DNA into a virus or its genomic DNA, and
(iv) tranducing individual CD47 deficient erythroblast populations with said individually extracted DNA incorporated in a virus or its genomic DNA
(v) incubating said CD47 deficient erythroblast populations with macrophages to produce macrophages expressing tumor associated antigens,
(vi) administering to said subject in need thereof parenterally by infusion or injection a tumoricidally effective amount of at least one of said macrophages expressing tumor associated antigens.
